(19) United States
(12) Patent Application Publication
(10) Pub. No.: US 2005/0065326 A1
(43) Pub. Date: Mar. 24, 2005

(54) Antibodies that specifically bind to TR2

(75) Inventors: Craig A. Rosen, Laytonsville, MD (US); Steven M. Ruben, Brookeville, MD (US)

Correspondence Address: HUMAN GENOME SCIENCES INC;INTELLECTUAL PROPERTY DEPT., 14200 SHADY GROVE ROAD, ROCKVILLE, MD, 20850, US

(73) Assignee: Human Genome Sciences, Inc., Rockville, MD (US)

(21) Appl. No.: 10/939,359

(22) Filed: Sep. 14, 2004

Related U.S. Application Data
(63) Continuation-in-part of application No. PCT/US03/10955, filed on Apr. 10, 2003.
(60) Provisional application No. 60/371,722, filed on Apr. 12, 2002.

(51) Int. Cl. C07H 21/04; C07K 16/28; C12N 5/06

(52) U.S. Cl. 530/388.22; 435/69.1; 435/320.1; 536/23.53; 435/334

(57) ABSTRACT

The present invention relates to antibodies and related molecules that specifically bind to TR2 proteins. Such antibodies have uses, for example, in the prevention and treatment of cancers and other proliferative disorders, autoimmune disorders, immunodeficiencies and/or HSV infection. The invention also relates to nucleic acid molecules encoding anti-TR2 antibodies, vectors and host cells containing these nucleic acids, and methods for producing the same. The present invention relates to methods and compositions for preventing, detecting, diagnosing, treating or ameliorating a disease or disorder, especially cancer and other hyperproliferative disorders, comprising administering to an animal, preferably a human, an effective amount of one or more antibodies or fragments or variants thereof, or related molecules, that specifically bind to TR2.

### CROSS-REFERENCE TO RELATED APPLICATIONS

This application is a continuation-in-part of PCT/US03/10955, filed Apr. 10, 2003, which in turn claims benefit under 35 U.S.C. § 119(e) of U.S. Provisional Application No. 60/371,722, filed Apr. 12, 2002, each of which applications is hereby incorporated by reference in its entirety.

### FIELD OF THE INVENTION

The present invention relates to antibodies and related molecules that specifically bind to TR2 proteins. Such antibodies have uses, for example, in the prevention and treatment of graft rejection and graft-versus-host disease, Herpes Simplex Virus (HSV) infection, autoimmune disorders, and cancer. The invention also relates to nucleic acid molecules encoding anti-TR2 antibodies, vectors and host cells containing these nucleic acids, and methods for producing the same. The present invention relates to methods and compositions for preventing, detecting, diagnosing, treating or ameliorating a disease or disorder, especially cancer and other hyperproliferative disorders, HSV infection, and autoimmune disorders, comprising administering to an animal, preferably a human, an effective amount of one or more antibodies or fragments or variants thereof, or related molecules, that specifically bind to TR2 proteins.

### BACKGROUND OF THE INVENTION

Human tumor necrosis factors a (TNF-α) and β (TNF-β or lymphotoxin) are related members of a broad class of polypeptide mediators, which includes the interferons, interleukins and growth factors, collectively called cytokines (Beutler, B. and Cerami, A., Annu. Rev. Immunol. 7:625-655 (1989)).

Tumor necrosis factor (TNF-α and TNF-β) was originally discovered as a result of its anti-tumor activity, however, now it is recognized as a pleiotropic cytokine playing important roles in a host of biological processes and pathologies. To date, there are at least 17 known members of the TNF-related cytokine family, including TNF-α, TNF-β (lymphotoxin-α), LTβ, TRAIL, Fas ligand, CD30, CD27, CD40, OX40 and 4-1BB receptors. These proteins have conserved C-terminal sequences and variable N-terminal sequences which are often used as membrane anchors, with the exception of TNF-β. Both TNF-α and TNF-β function as homotrimers when they bind to TNF receptors.

TNF is produced by a number of cell types, including monocytes, fibroblasts, T-cells, natural killer (NK) cells and predominately by activated macrophages. TNF-α has been reported to have a role in the rapid necrosis of tumors, immunostimulation, autoimmune disease, graft rejection, producing an anti-viral response, septic shock, cerebral malaria, cytotoxicity, protection against deleterious effects of ionizing radiation produced during a course of chemotherapy, such as denaturation of enzymes, lipid peroxidation and DNA damage (Nata et al., J. Immunol. 136(7):2483 (1987)), growth regulation, vascular endothelium effects and metabolic effects. TNF-α also triggers endothelial cells to secrete various factors, including PAI-1, IL-1, GM-CSF and IL-6 to promote cell proliferation. In addition, TNF-α up-regulates various cell adhesion molecules such as E-Selectin, ICAM-1 and VCAM-1. TNF-α and the Fas ligand have also been shown to induce programmed cell death. TNF-β has many activities, including induction of an antiviral state and tumor necrosis, activation of polymorphonuclear leukocytes, induction of class I major histocompatibility complex antigens on endothelial cells, induction of adhesion molecules on endothelium and growth hormone stimulation (Ruddle, N. and Homer, R., Prog. Allergy 40:162-182 (1988)).

Both TNF-α and TNF-β are involved in growth regulation and interact with hemopoietic cells at several stages of differentiation, inhibiting proliferation of various types of precursor cells, and inducing proliferation of immature myelomonocytic cells. Porter, A., Tibtech 9:158-162 (1991).

Recent studies with “knockout” mice have shown that mice deficient in TNF-β production show abnormal development of the peripheral lymphoid organs and morphological changes in spleen architecture (reviewed in Aggarwal et al., Eur Cytokine Netw, 7(2):93-124 (1996)). With respect to the lymphoid organs, the popliteal, inguinal, para-aortic, mesenteric, axillary and cervical lymph nodes failed to develop in TNF-β−/− mice. In addition, peripheral blood from TNF-β−/− mice contained a three fold reduction in white blood cells as compared to normal mice. Peripheral blood from TNF-β−/− mice, however, contained four fold more B cells as compared to their normal counterparts. Further, TNF-β, in contrast to TNF-α has been shown to induce proliferation of EBV-infected B cells. These results indicate that TNF-β, is involved in lymphocyte development.

The first step in the induction of the various cellular responses mediated by TNF-α or TNF-β is their binding to specific cell surface or soluble receptors. Two distinct TNF receptors of approximately 55-KDa (TNF-RI) and 75-KDa (TNF-RII) have been identified (Hohman et al., J. Biol. Chem., 264:14927-14934 (1989)), and human and mouse cDNAs corresponding to both receptor types have been isolated and characterized (Loetscher et al., Cell, 61:351 (1990)). Both TNF-Rs share the typical structure of cell surface receptors including extracellular, transmembrane and intracellular regions.

These molecules exist not only in cell bound forms, but also in soluble forms, consisting of the cleaved extra-cellular domains of the intact receptors (Nophar et al., EMBO Journal, 9(10):3269-76 (1990)) and otherwise intact receptors wherein the transmembrane domain is lacking. The extracellular domains of TNF-RI and TNF-RII share 28% identity and are characterized by four repeated cysteine-rich motifs with significant intersubunit sequence homology. The majority of cell types and tissues appear to express both TNF receptors and both receptors are active in signal transduction, however, they are able to mediate distinct cellular responses. Further, TNF-RII was shown to exclusively mediate human T-cell proliferation by TNF as shown in PCT WO 94/09137.

TR2 (TNFR-related 2; also known as herpesvirus entry mediator (HVEM) and another TRAF-associated receptor (ATAR)) is a recently identified receptor of the TNF-receptor superfamily with homology to TNF-RII (Kwon, B. S. et al., J. Biol. Chem. 272:14272-14276 (1997), International Patent Application Nos. WO96/34095, WO98/18824, WO98/25967, WO99/35170, WO00/56405, SEQ ID: NOs:1, 3, and 5). Each of the publications and patents cited above is hereby incorporated by reference in their entireties, particularly with respect to the nucleotide and amino acid sequences of the TR2 disclosed therein.

Along with other members of the TNFR family, TR2 contains the characteristic cysteine-rich repeats in its extracellular domain. However, the TR2 cytoplasmic tail does not contain the death domain seen in the Fas and TNF-RI intracellular domains and therefore appears more closely related to the intracellular domains of CD40 and 4-1BB. Signals through 4-1BB and CD40 have been shown to be co-stimulatory to T and B cells, respectively (Banchereau, J. and Rousset, F., Nature 353:678-679 (1991); Hurtado, J. et al., J. Immunol. 158:2600-2609 (1997)). Mapping studies indicate that the TR2 gene is located on the chromosome 1 region p36.2-p36.3, which is in close proximity with CD30, 4-1BB, OX-40, and TNF-RII genes (Kwon, B. S. et al., J. Biol. Chem. 272:14272-14276 (1997)). Interestingly, all of these receptors have been shown to stimulate T cells upon cognate ligand binding, in contrast to Fas and TNF-RI, which stimulate apoptosis.

TR2 is expressed mainly in hemopoetic tissues, is high in lymphoid tissues such as spleen and thymus, and is expressed moderately in bone marrow and small intestine. TR2 expression is also detected in purified primary reseting and activated CD4 and CD8 positive T cells, and CD19 positive B lymphocytes and monocytes (Kwon, B. S., et al. (1997), supra). Overexpression studies suggest that TR2 may activate NF-kB and AP-1 signalling pathways (Hsu, H. et al., J. Biol. Chem. 272:13471-13474 (1997); Marsters, S. A. et al., J. Biol. Chem. 272:14029-14032 (1997)). Furthermore, results suggest that TR2 is involved in T lymphocyte activation and differentiation (Harrop, J. A. et al. J. Immunol. 161:1786-1794 (1998)). At least two TR2 ligands have been identified, including TL5 (also referred to as LIGHT; International Publication Nos. WO 97/34911 and WO 99/42584; Zhai et al., J. Clin. Invest. 102:1142-1151 (1998); SEQ ID NO:45), and LTα (also referred to as lymphotoxin-α and TNF-β; Granger et al., Nature 218:1253-4 (1968); SEQ ID NO:46). The disclosures of each of the above-cited patents, applications, and references are hereby incorporated by reference in their entirety.

The effects of TNF family ligands and TNF family receptors are varied and influence numerous functions, both normal and abnormal, in the biological processes of the mammalian system. There is a clear need, therefore, for identification and characterization of compositions, such as antibodies, that influence the biological activity of TNF receptors, both normally and in disease states. In particular, there is a need to isolate and characterize antibodies that modulate the biological activities of TR2 proteins.

### SUMMARY OF THE INVENTION

The present invention encompasses antibodies (including molecules comprising, or alternatively consisting of, antibody fragments or variants thereof) that specifically bind to a TR2 polypeptide or polypeptide fragment or variant of TR2. In particular, the invention encompasses antibodies (including molecules comprising, or alternatively consisting of, antibody fragments or variants thereof) that specifically bind to a polypeptide or polypeptide fragment or variant of human TR2 such as those of SEQ ID NO:2, SEQ ID NO:4, or SEQ ID NO:6. The invention also encompasses antibodies (including molecules comprising, or alternatively consisting of, antibody fragments or variants thereof) that specifically bind to allelic variants of TR2 proteins including, but not limited to, either guanine or adenine allelic variants in which nucleotide 314 (numbering according to the numbering of SEQ ID NO:1) contains either guanine (guanylic acid) or adenine (adenylic acid) and/or in which nucleotides 386, 624 and 627 contain either thymine (thymidylic acid) or cytosine (cytidylic acid) shown in SEQ ID NO:1. The invention also encompasses antibodies that specifically bind to TR2 proteins that are expressed on the surface of a cell.

The present invention relates to methods and compositions for preventing, treating or ameliorating a disease or disorder comprising administering to an animal, preferably a human, an effective amount of one or more antibodies or fragments or variants thereof, or related molecules, that specifically bind to a TR2 polypeptide or a fragment or variant thereof. In specific embodiments, the present invention relates to methods and compositions for preventing, treating or ameliorating a disease or disorder associated with TR2 function or TR2 ligand function or aberrant TR2 or TR2 ligand expression, comprising administering to an animal, preferably a human, an effective amount of one or more antibodies or fragments or variants thereof, or related molecules, that specifically bind to a TR2 polypeptide or a fragment or variant thereof.

In highly preferred embodiments, the present invention relates to antibody-based methods and compositions for preventing, treating or ameliorating cancers and other hyperproliferative disorders (e.g., leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors.

In other highly preferred embodiments, the present invention relates to antibody-based methods and compositions for preventing, treating or ameliorating autoimmune disease or conditions associated with an autoimmune disease. In a specific preferred embodiment, rheumatoid arthritis is treated, prevented, prognosed and/or diagnosed using antibodies of the invention. In another specific preferred embodiment, autoimmune diseases in which T cell responses, especially aberrant Type I Tₕₑₗₚₑᵣ or T_{ctl} responses play a role in the pathology, (e.g., multiple sclerosis, myasthenia gravis).

In other highly preferred embodiments, the present invention relates to antibody-based methods and compositions for preventing, treating or ameliorating immunodeficiency or conditions associated with an immunodeficiency.

Other diseases and disorders which can be treated, prevented or ameliorated with the antibodies of the invention include, but are not limited to, inflammatory disorders (e.g., asthma, allergic disorders, graft-versus-host disease) and infectious diseases (e.g., AIDS, herpes viral infections, and other viral infections).

The present invention also encompasses methods and compositions for detecting, diagnosing, or prognosing diseases or disorders comprising administering to an animal, preferably a human, an effective amount of one or more antibodies or fragments or variants thereof, or related molecules, that specifically bind to TR2 or a fragment or variant thereof. In specific embodiments, the present invention also encompasses methods and compositions for detecting, diagnosing, or prognosing diseases or disorders associated with TR2 function or TR2 ligand function or aberrant TR2 or TR2 ligand expression, comprising administering to an animal, preferably a human, an effective amount of one or more antibodies or fragments or variants thereof, or related molecules, that specifically bind to TR2 or a fragment or variant thereof. In highly preferred embodiments, the present invention relates to antibody-based methods and compositions for detecting, diagnosing, or prognosing cancers and other hyperproliferative disorders (e.g., leukemia, carcinoma, and lymphoma). Other diseases and disorders which can be detected, diagnosed or prognosed with the antibodies of the invention include, but are not limited to, immune disorders (e.g., graft-versus-host disease, lupus, rheumatoid arthritis, multiple sclerosis, myasthenia gravis, Hashimoto's disease, and immunodeficiency syndrome), inflammatory disorders (e.g., asthma, allergic disorders), and infectious diseases (e.g., AIDS, herpes viral infections, and other viral infections).

In one embodiment, antibodies of the invention are used to prevent, diagnose, treat or ameliorate a disease or disorder associated with aberrant T cell (e.g., Th1 or CD8+ T cells) activity, for example, aberrant T cell proliferation, differentiation, or effector function. In a specific embodiment, antibodies of the invention are used to prevent, diagnose, treat or ameliorate a disease or disorder associated with increased or excess T cell (e.g., Th1 or CD8+ T cells) activity, for example, increased or excess T cell proliferation, differentiation, or effector function.

Another embodiment of the present invention includes the use of the antibodies of the invention as a diagnostic tool to monitor the expression of TR2 expression on cells.

The present inventors have generated single chain Fv's (scFvs) that specifically bind TR2 polypeptides (e.g., SEQ ID NO:8). Thus, the invention encompases these scFvs, listed in Table 1. In addition the invention encomasses cell lines engineered to express antibodies corresponding to these scFvs which have been deposited with the American Type Culture Collection (“ATCC”) as of the dates listed in Table 1 and given the ATCC Deposit Numbers identified in Table 1 The ATCC is located at 10801 University Boulevard, Manassas, Va. 20110-2209, USA. The ATCC deposit was made pursuant to the terms of the Budapest Treaty on the International Recognition of the Deposit of Microorganisms for Purposes of Patent Procedure.

Further, the present invention encompasses the polynucleotides encoding the scFvs, as well as the amino acid sequences encoding the scFvs. Molecules comprising, or alternatively consisting of, fragments or variants of these scFvs (e.g., VH domains, VH CDRs, VL domains, or VL CDRs having an amino acid sequence of the corresponding portion of any one of the scFvs referred to in Table 1), that specifically bind to TR2 or fragments or variants thereof are also encompassed by the invention, as are nucleic acid molecules that encode these antibodies and/or molecules. In highly preferred embodiments, the present invention encompasses antibodies, or fragments or variants thereof, that bind to the extracellular regions/domains of TR2 proteins or fragments and variants thereof.

The present invention also provides antibodies that bind TR2 polypeptides which are coupled to a detectable label, such as an enzyme, a fluorescent label, a luminescent label, or a bioluminescent label. The present invention also provides antibodies that bind TR2 polypeptides which are coupled to a therapeutic or cytotoxic agent. The present invention also provides antibodies that bind TR2 polypeptides which are coupled to a radioactive material.

The present invention also provides antibodies that specifically bind one or more TR2 polypeptides that act as either TR2 agonists or TR2 antagonists. In specific embodiments, the antibodies of the invention inhibit proliferation of TR2 expressing cells. In specific embodiments, the antibodies of the invention inhibit differentiation of TR2 expressing cells (e.g., T-cells). In specific embodiments, the antibodies of the invention inhibit Herpes Simplex Virus (HSV) infection of TR2 expressing cells. In other specific embodiments, the antibodies of the invention inhibit TR2 ligand binding to TR2. In other specific embodiments, the antibodies of the invention downregulate TR2 expression.

The present invention also provides antibodies that stimulate proliferation of TR2 expressing cells. The present invention also provides antibodies that stimulate differentiation of TR2 expressing cells (e.g. T-cells). In other specific embodiments, the antibodies of the invention upregulate TR2 expression.

In further embodiments, the antibodies of the invention have a dissociation constant (K_{D}) of 10⁻⁷ M or less. In preferred embodiments, the antibodies of the invention have a dissociation constant (K_{D}) of 10⁻⁹ M or less.

The present invention further provides antibodies that stimulate proliferation and/or differentiation of TR2 expressing cells equally well in the presence or absence of antibody cross-linking reagents; and/or stimulate proliferation and/or differentiation with equal or greater potency as an equal concentration of anti-TR2 antibody or TR2 ligand in the absence of a cross-linking antibody or other cross-linking agent.

In further embodiments, antibodies of the invention have an off rate (k_{off}) of 10⁻³/sec or less. In preferred embodiments, antibodies of the invention have an off rate (k_{off}) of 10⁻⁴/sec or less. In other preferred embodiments, antibodies of the invention have an off rate (k_{off}) of 10⁻⁵/sec or less.

The present invention also provides for antibodies that preferentially bind one or more of the TR2 proteins selected from the group of TR2, TR2-SV1, and TR2-SV2.

The present invention also provides panels of antibodies (including molecules comprising, or alternatively consisting of, antibody fragments or variants) wherein the panel members correspond to one, two, three, four, five, ten, fifteen, twenty, or more different antibodies of the invention (e.g., whole antibodies, Fabs, F(ab′)₂ fragments, Fd fragments, disulfide-linked Fvs (sdFvs), anti-idiotypic (anti-Id) antibodies, and scFvs). The present invention further provides mixtures of antibodies, wherein the mixture corresponds to one, two, three, four, five, ten, fifteen, twenty, or more different antibodies of the invention (e.g., whole antibodies, Fabs, F(ab′)₂ fragments, Fd fragments, disulfide-linked Fvs (sdFvs), anti-idiotypic (anti-Id) antibodies, and scFvs)). The present invention also provides for compositions comprising, or alternatively consisting of, one, two, three, four, five, ten, fifteen, twenty, or more antibodies of the present invention (including molecules comprising, or alternatively consisting of, antibody fragments or variants thereof). A composition of the invention may comprise, or alternatively consist of, one, two, three, four, five, ten, fifteen, twenty, or more amino acid sequences of one or more antibodies or fragments or variants thereof. Alternatively, a composition of the invention may comprise, or alternatively consist of, nucleic acid molecules encoding one or more antibodies of the invention.

The present invention also provides for fusion proteins comprising an antibody (including molecules comprising, or alternatively consisting of, antibody fragments or variants thereof) of the invention, and a heterologous polypeptide (i.e., a polypeptide unrelated to an antibody or antibody domain). Nucleic acid molecules encoding these fusion proteins are also encompassed by the invention. A composition of the present invention may comprise, or alternatively consist of, one, two, three, four, five, ten, fifteen, twenty or more fusion proteins of the invention. Alternatively, a composition of the invention may comprise, or alternatively consist of, nucleic acid molecules encoding one, two, three, four, five, ten, fifteen, twenty or more fusion proteins of the invention.

The present invention also provides for a nucleic acid molecule(s), generally isolated, encoding an antibody (including molecules, such as scFvs, VH domains, or VL domains, that comprise, or alternatively consist of, an antibody fragment or variant thereof) of the invention. The present invention also provides a host cell transformed with a nucleic acid molecule of the invention and progeny thereof. The present invention also provides a method for the production of an antibody (including a molecule comprising, or alternatively consisting of, an antibody fragment or variant thereof) of the invention. The present invention further provides a method of expressing an antibody (including a molecule comprising, or alternatively consisting of, an antibody fragment or variant thereof) of the invention from a nucleic acid molecule. These and other aspects of the invention are described in further detail below.

### DETAILED DESCRIPTION OF THE INVENTION

Definitions

The term “antibody,” as used herein, refers to immunoglobulin molecules and immunologically active portions of immunoglobulin molecules, i.e., molecules that contain an antigen binding site that specifically binds an antigen. As such, the term antibody encompasses not only whole antibody molecules, but also antibody multimers and antibody fragments as well as variants (including derivatives) of antibodies, antibody multimers and antibody fragments. Examples of molecules which are described by the term “antibody” herein include, but are not limited to: single chain Fvs (scFvs), Fab fragments, Fab′ fragments, F(ab′)₂, disulfide linked Fvs (sdFvs), Fvs, and fragments comprising or alternatively consisting of, either a VL or a VH domain. The term “single chain Fv” or “scFv” as used herein refers to a polypeptide comprising a VH domain of antibody linked to a VL domain of an antibody. Antibodies that specifically bind to TR2 may have cross-reactivity with other antigens. Preferably, antibodies that specifically bind to TR2 do not cross-react with other antigens (e.g., other members of the Tumor Necrosis Factor Receptor superfamily). Antibodies that specifically bind to TR2 can be identified, for example, by immunoassays or other techniques known to those of skill in the art, e.g., immunoassays.

Antibodies of the invention include, but are not limited to, monoclonal, multispecific, human or chimeric antibodies, single chain antibodies, Fab fragments, F(ab′) fragments, anti-idiotypic (anti-Id) antibodies (including, e.g., anti-Id antibodies to antibodies of the invention), intracellularly-made antibodies (i.e., intrabodies), and epitope-binding fragments of any of the above. The immunoglobulin molecules of the invention can be of any type (e.g., IgG, IgE, IgM, IgD, IgA and IgY), class (e.g., IgG₁, IgG₂, IgG₃, IgG₄, IgA₁ and IgA₂) or subclass of immunoglobulin molecule. Preferably, an antibody of the invention comprises, or alternatively consists of, a VH domain, VH CDR, VL domain, or VL CDR having an amino acid sequence of any one of those referred to in Table 1, or a fragment or variant thereof. In a preferred embodiment, the immunoglobulin is an IgG1 isotype. In another preferred embodiment, the immunoglobulin is an IgG4 isotype. Immunoglobulins may have both a heavy and light chain. An array of IgG, IgE, IgM, IgD, IgA, and IgY heavy chains may be paired with a light chain of the kappa or lambda forms.

Antibodies of the invention may also include multimeric forms of antibodies. For example, antibodies of the invention may take the form of antibody dimers, trimers, or higher-order multimers of monomeric immunoglobulin molecules. Dimers of whole immunoglobulin molecules or of F(ab′)2 fragments are tetravalent, whereas dimers of Fab fragments or scFv molecules are bivalent. Individual monomers withon an antibody multimer may be identical or different, i.e., they may be heteromeric or homomeric antibody multimers. For example, individual antibodies within a multimer may have the same or different binding specificities.

Multimerization of antibodies may be accomplished through natural aggregation of antibodies or through chemical or recombinant linking techniques known in the art. For example, some percentage of purified antibody preparations (e.g., purified IgG1 molecules) spontaneously form protein aggregates containing antibody homodimers, and other higher-order antibody multimers. Alternatively, antibody homodimers may be formed through chemical linkage techniques known in the art. For example, heterobifunctional crosslinking agents including, but not limited to, SMCC [succinimidyl 4-(maleimidomethyl)cyclohexane-1-carboxylate] and SATA [N-succinimidyl S-acethylthio-acetate] (available, for example, from Pierce Biotechnology, Inc. (Rockford, Ill.)) can be used to form antibody multimers. An exemplary protocol for the formation of antibody homodimers is given in Ghetie et al., Proceedings of the National Academy of Sciences USA (1997) 94:7509-7514, which is hereby incorporated by reference in its entirety. Antibody homodimers can be converted to Fab′2 homodimers through digestion with pepsin. Another way to form antibody homodimers is through the use of the autophilic T15 peptide described in Zhao and Kohler, The Journal of Immunology (2002) 25:396-404, which is hereby incorporated by reference in its entirety.

Alternatively, antibodies can be made to multimerize through recombinant DNA techniques. IgM and IgA naturally form antibody multimers through the interaction with the J chain polypeptide. Non-IgA or non-IgM molecules, such as IgG molecules, can be engineered to contain the J chain interaction domain of IgA or IgM, thereby conferring the ability to form higher order multimers on the non-IgA or non-IgM molecules. (see, for example, Chintalacharuvu et al., (2001) Clinical Immunology 101:21-31. and Frigerio et al., (2000) Plant Physiology 123:1483-94., both of which are hereby incorporated by reference in their entireties.) ScFv dimers can also be formed through recombinant techniques known in the art; an example of the construction of scFv dimers is given in Goel et al., (2000) Cancer Research 60:6964-6971 which is hereby incorporated by reference in its entirety. Antibody multimers may be purified using any suitable method known in the art, including, but not limited to, size exclusion chromatography.

By “isolated antibody” is intended an antibody removed from its native environment. Thus, for example, an antibody produced by, purified from and/or contained within a hybridoma and/or a recombinant host cell is considered isolated for purposes of the present invention.

Unless otherwise defined in the specification, specific binding or immunospecifc binding by an anti-TR2 antibody means that the anti-TR2 antibody binds TR2 but does not significantly bind to (i.e., cross react with) proteins other than TR2, such as other proteins in the same family of proteins). An antibody that binds TR2 protein and does not cross-react with other proteins is not necessarily an antibody that does not bind said other proteins in all conditions; rather, the TR2-specific antibody of the invention preferentially binds TR2 compared to its ability to bind said other proteins such that it will be suitable for use in at least one type of assay or treatment, i.e., give low background levels or result in no unreasonable adverse effects in treatment. It is well known that the portion of a protein bound by an antibody is known as the epitope. An epitope may either be linear (i.e., comprised of sequential amino acids residues in a protein sequences) or conformational (i.e., comprised of one or more amino acid residues that are not contiguous in the primary structure of the protein but that are brought together by the secondary, tertiary or quaternary structure of a protein). Given that TR2-specific antibodies bind to epitopes of TR2, an antibody that specifically binds TR2 may or may not bind fragments of TR2 and/or variants of TR2 (e.g., proteins that are at least 90% identical to TR2) depending on the presence or absence of the epitope bound by a given TR2-specific antibody in the TR2 fragment or variant. Likewise, TR2-specific antibodies of the invention may bind species orthologues of TR2 (including fragments thereof) depending on the presence or absence of the epitope recognized by the antibody in the orthologue. Additionally, TR2-specific antibodies of the invention may bind modified forms of TR2, for example, TR2 fusion proteins. In such a case when antibodies of the invention bind TR2 fusion proteins, the antibody must make binding contact with the TR2 moiety of the fusion protein in order for the binding to be specific. Antibodies that specifically bind to TR2 can be identified, for example, by immunoassays or other techniques known to those of skill in the art, e.g., the immunoassays described in the Examples below.

The term “variant” as used herein refers to a polypeptide that possesses a similar or identical amino acid sequence as a TR2 polypeptide, a fragment of a TR2 polypeptide, an anti-TR2 antibody or antibody fragment thereof. A variant having a similar amino acid sequence refers to a polypeptide that satisfies at least one of the following: (a) a polypeptide comprising, or alternatively consisting of, an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the amino acid sequence of a TR2 polypeptide, a fragment thereof, an anti-TR2 antibody or antibody fragment thereof (including a VH domain, VHCDR, VL domain, or VLCDR having an amino acid sequence of the corresponding part of an antibody expressed by any one or more cell lines referred to in Table 1) described herein; (b) a polypeptide encoded by a nucleotide sequence, the complementary sequence of which hybridizes under stringent conditions to a nucleotide sequence encoding a TR2 polypeptide (e.g., SEQ ID NOS:2, 4 and/or 6), a fragment of a TR2 polypeptide, an anti-TR2 antibody or antibody fragment thereof (including a VH domain, VHCDR, VL domain, or VLCDR having an amino acid sequence of the corresponding part of an antibody expressed by any one or more cell lines referred to in Table 1), described herein, of at least 5 amino acid residues, at least 10 amino acid residues, at least 15 amino acid residues, at least 20 amino acid residues, at least 25 amino acid residues, at least 30 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues; and (c) a polypeptide encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99%, identical to the nucleotide sequence encoding a TR2 polypeptide, a fragment of a TR2 polypeptide, an anti-TR2 antibody or antibody fragment thereof (including a VH domain, VHCDR, VL domain, or VLCDR having an amino acid sequence of the corresponding part of an antibody expressed by any one or more cell lines referred to in Table 1), described herein. A polypeptide with similar structure to a TR2 polypeptide, a fragment of a TR2 polypeptide, an anti-TR2 antibody or antibody fragment thereof, described herein refers to a polypeptide that has a similar secondary, tertiary or quaternary structure of a TR2 polypeptide, a fragment of a TR2 polypeptide, an anti-TR2 antibody, or antibody fragment thereof, described herein. The structure of a polypeptide can determined by methods known to those skilled in the art, including but not limited to, X-ray crystallography, nuclear magnetic resonance, and crystallographic electron microscopy. Preferably, a variant TR2 polypeptide, a variant fragment of a TR2 polypeptide, or a variant anti-TR2 antibody and/or antibody fragment possesses similar or identical function and/or structure as the reference TR2 polypeptide, the reference fragment of a TR2 polypeptide, or the reference anti-TR2 antibody and/or antibody fragment, respectively.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide at the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The determination of percent identity between two sequences can be accomplished using a mathematical algorithm known to those of skill in the art. An example of a mathematical algorithm for comparing two sequences is the algorithm of Karlin and Altschul Proc. Natl. Acad. Sci. USA 87:2264-2268(1990), modified as in Karlin and Altschul Proc. Natl. Acad. Sci. USA 90:5873-5877(1993). The BLASTn and BLASTx programs of Altschul, et al. J. Mol. Biol. 215:403-410(1990) have incorporated such an alogrithm. BLAST nucleotide searches can be performed with the BLASTn program (score=100, wordlength=12) to obtain nucleotide sequences homologous to a nucleic acid molecules of the invention. BLAST protein searches can be performed with the BLASTx program (score=50, wordlength=3) to obtain amino acid sequences homologous to a protein molecules of the invention. To obtain gapped alignments for comparison purposes, Gapped BLAST can be utilized as described in Altschul et al. Nucleic Acids Res. 25:3589-3402(1997). Alternatively, PSI-BLAST can be used to perform an iterated search which detects distant relationships between molecules (Id.). When utilizing BLAST, Gapped BLAST, and PSI-BLAST programs, the default parameters of the respective programs (e.g., BLASTx and BLASTn) can be used.

Another example of a mathematical algorithm utilized for the comparison of sequences is the algorithm of Myers and Miller, CABIOS (1989). The ALIGN program (version 2.0) which is part of the GCG sequence alignment software package has incorporated such an alogrithm. Other algorithms for sequence analysis known in the art include ADVANCE and ADAM as described in Torellis and Robotti Comput. Appl. Biosci., 10 :3-5(1994); and FASTA described in Pearson and Lipman Proc. Natl. Acad. Sci. 85:2444-8(1988). Within FASTA, ktup is a control option that sets the sensitivity and speed of the search.

The term “derivative” as used herein, refers to a variant polypeptide of the invention that comprises, or alternatively consists of, an amino acid sequence of a TR2 polypeptide, a fragment of a TR2 polypeptide, or an antibody of the invention that specifically binds to a TR2 polypeptide, which has been altered by the introduction of amino acid residue substitutions, deletions or additions. The term “derivative” as used herein also refers to a TR2 polypeptide, a fragment of a TR2 polypeptide, or an antibody that specifically binds to a TR2 polypeptide which has been modified, e.g., by the covalent attachment of any type of molecule to the polypeptide. For example, but not by way of limitation, a TR2 polypeptide, a fragment of a TR2 polypeptide, or an anti-TR2 antibody, may be modified, e.g., by glycosylation, acetylation, pegylation, phosphorylation, amidation, derivatization by known protecting/blocking groups, proteolytic cleavage, linkage to a cellular ligand or other protein, etc. A derivative of a TR2 polypeptide, a fragment of a TR2 polypeptide, or an anti-TR2 antibody, may be modified by chemical modifications using techniques known to those of skill in the art, including, but not limited to, specific chemical cleavage, acetylation, formylation, metabolic synthesis of tunicamycin, etc. Further, a derivative of a TR2 polypeptide, a fragment of a TR2 polypeptide, or an anti-TR2 antibody, may contain one or more non-classical amino acids. A polypeptide derivative possesses a similar or identical function as a TR2 polypeptide, a fragment of a TR2 polypeptide, or an anti-TR2 antibody, described herein.

The term “fragment” as used herein refers to a polypeptide comprising an amino acid sequence of at least 5 amino acid residues, at least 10 amino acid residues, at least 15 amino acid residues, at least 20 amino acid residues, at least 25 amino acid residues, at least 30 amino acid residues, at least 35 amino acid residues, at least 40 amino acid residues, at least 45 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, at least 150 amino acid residues, at least 175 amino acid residues, at least 200 amino acid residues, or at least 250 amino acid residues, of the amino acid sequence of TR2, or an anti-TR2 antibody (including molecules such as scFv's, that comprise, or alternatively consist of, antibody fragments or variants thereof) that specifically binds to TR2.

The term “host cell” as used herein refers to the particular subject cell transfected with a nucleic acid molecule and the progeny or potential progeny of such a cell. Progeny may not be identical to the parent cell transfected with the nucleic acid molecule due to mutations or environmental influences that may occur in succeeding generations or integration of the nucleic acid molecule into the host cell genome.

As used herein the phrase “splice variant” refers to cDNA molecules produced from RNA molecules initially transcribed from the same genomic DNA sequence which have undergone alternative RNA splicing. Alternative RNA splicing occurs when a primary RNA transcript undergoes splicing, generally for the removal of introns, which results in the production of more than one type of mRNA molecule each of which may encode different amino acid sequences. The term “splice variant” also refers to the proteins encoded by the above cDNA molecules.

Unless indicated, “TR2 proteins”, “TR2”, proteins“and “TR2 polypeptides” refer to all TR2 polypeptides or fragments or variants thereof as well as proteins resulting from the alternate splicing of the genomic DNA sequences encoding proteins having regions of amino acid sequence identity and receptor activity which correspond to the proteins shown in SEQ ID NO:2, SEQ ID NO:4 or SEQ ID NO:6, as well as TR2 allelic variants.

Antibody Structure

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 10 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgG, IgA, and IgE, respectively. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site.

Thus, an intact IgG antibody has two binding sites. Except in bifunctional or bispecific antibodies, the two binding sites are the same.

The chains all exhibit the same general structure of relatively conserved framework regions (FR) joined by three hyper variable regions, also called complementarity determining regions or CDRs. The CDRs from the heavy and the ligt chains of each pair are aligned by the framework regions, enabling binding to a specific epitope. From N-terminal to C-terminal, both light and heavy chains comprise the domains FRI, CDR1, FR2, CDR2, FR3, CDR3 and FR4. The assignment of amino acids to each domain is in accordance with the definitions of Kabat Sequences of Proteins of Immunological Interest (National Institutes of Health, Bethesda, Md. (1987 and 1991)), or Chothia & Lesk J Mol. Biol. 196:901-917 (1987); Chothia et al. Nature 342:878-883 (1989).

A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann Clin. Exp. Immunol. 79: 315-321 (1990), Kostelny et al. J Immunol. 148:1547 1553 (1992). In addition, bispecific antibodies may be formed as “diabodies” (Holliger et al. “‘Diabodies’: small bivalent and bispecific antibody fragments” PNAS USA 90:6444-6448 (1993)) or “Janusins” (Traunecker et al. “Bispecific single chain molecules (Janusins) target cytotoxic lymphocytes on HIV infected cells” EMBO J 10:3655-3659 (1991) and Traunecker et al. “Janusin: new molecular design for bispecific reagents” Int. J. Cancer Suppl. 7:51-52 (1992)).

Production of bispecific antibodies can be a relatively labor intensive process compared with production of conventional antibodies and yields and degree of purity are generally lower for bispecific antibodies. Bispecific antibodies do not exist in the form of fragments having a single binding site (e.g., Fab, Fab′, and Fv).

Anti-TR2 Antibodies

Using phage display technology, single chain antibody molecules (“scFvs”) that specifically bind to TR2 proteins (or fragments or variants thereof) have been identified. Molecules comprising, or alternatively consisting of, fragments or variants of these scFvs (e.g., including VH domains, VH CDRs, VL domains, or VL CDRs having an amino acid sequence of any one of those referred to in Table 1), that specifically bind to TR2 proteins (or fragments or variants thereof) are also encompassed by the invention, as are nucleic acid molecules that encode these scFvs, and/or molecules.

In particular, the invention relates to scFvs comprising, or alternatively consisting of the amino acid of SEQ ID NO: 8, referred to in Table 1 below. Molecules comprising, or alternatively consisting of, fragments or variants of this scFv (e.g., including VH domains, VH CDRs, VL domains, or VL CDRs having an amino acid sequence in Table 1), that specifically bind to TR2 proteins are also encompassed by the invention, as are nucleic acid molecules that encode these scFvs, and/or molecules (e.g., SEQ ID NO:7).

The ScFv corresponding to SEQ ID NO:8 was selected for its ability bind TR2 polypeptide.

The present invention provides antibodies (including molecules comprising, or alternatively consisting of, antibody fragments or variants thereof) that specifically bind to a polypeptide or a polypeptide fragment of TR2. In particular, the invention provides antibodies corresponding to the scFvs referred to in Table 1, such scFvs may routinely be “converted” to immunoglobulin molecules by inserting, for example, the nucleotide sequences encoding the VH and/or VL domains of the scFv into an expression vector containing the constant domain sequences and engineered to direct the expression of the immunoglobulin molecule, as described in more detail in Example 2 below.

Cell lines that express IgG1 antibodies that comprise the VH and VL domains of scFvs of the invention have been deposited with the American Type Culture Collection (“ATCC”) on the dates listed in Table 1 and given the ATCC Deposit Numbers identified in Table 1. The ATCC is located at 10801 University Boulevard, Manassas, Va. 20110-2209, USA. The ATCC deposit was made pursuant to the terms of the Budapest Treaty on the international recognition of the deposit of microorganisms for purposes of patent procedure.

Accordingly, in one embodiment, the invention provides antibodies that comprise the VH and VL domains of scFvs of the invention.

In a preferred embodiment, an antibody of the invention is the antibody expressed by cell line X (See Table 1).

TABLE 1
scFvs that Specifically bind to TR2 proteins

| | scFv | scFv | | | | | | | | | | | |
|---|---|---|---|---|---|---|---|---|---|---|---|---|---|
| | DNA | Protein | AAs of | AAs of | AAs of | AAs of | AAs of | AAs of | AAs of | AAs of | Cell Line | ATCC | ATCC |
| | SEQ ID | SEQ ID | VH | VH | VH | VH | VL | VL | VL | VL | Expressing | Deposit | Deposit |
| scFv | NO: | NO: | Domain | CDR1 | CDR2 | CDR3 | Domain | CDR1 | CDR2 | CDR3 | antibody | Number | Date |
| TR01G03 | 7 | 8 | 1-121 | 26-35 | 50-66 | 99-110 | 139-246 | 162-172 | 188-194 | 227-235 | X | Y | |

The present invention encompasses antibodies (including molecules comprising, or alternatively consisting of, antibody fragments or variants thereof) that specifically bind to a TR2 polypeptide or a fragment, variant, or fusion protein thereof. A TR2 polypeptide includes, but is not limited to, TR2 (SEQ ID NO:2) or the polypeptide encoded by the cDNA contained in ATCC Deposit 97059 deposited Feb. 13, 1995; TR2-SV1 (SEQ ID NO:4) or the polypeptide encoded by the cDNA contained in ATCC Deposit 97058 deposited Feb. 13, 1995; and/or TR2-SV2 (SEQ ID NO:6) or the polypeptide encoded by the cDNA contained in ATCC Deposit 97057 deposited Feb. 13, 1995. TR2 proteins may be produced through recombinant expression of nucleic acids encoding the polypeptides of SEQ ID NOS:2, 4, or 6, (e.g., the cDNAs in the ATCC Deposit Numbers 97059, 97058, or 97057). Antibodies of the invention may specifically bind TR2 proteins including, but not limited to, TR2, TR2-SV1, and TR2-SV2 as well as fragments and variants thereof, and are described in more detail below.

In one embodiment, the antibodies of the invention preferentially bind TR2 (SEQ ID NO:2), or fragments, variants, or fusion proteins thereof (e.g., the extracellular region of TR2 fused to an Fc domain) relative to their ability to bind TR2-SV1 or TR2-SV2 (SEQ ID NOS:4 and 6) or fragments, variants, or fusion proteins thereof.

In another embodiment, antibodies of the invention preferentially bind TR2-SV1 (SEQ ID NO:4), fragments, variants, or fusion proteins thereof relative to their ability to bind TR2, or TR2-SV2 (SEQ ID NOS:2 and 6) or fragments, variants, or fusion proteins thereof. In another embodiment, antibodies of the invention preferentially bind TR2-SV2, fragments, variants, or fusion proteins thereof relative to their ability to bind TR2, or TR2-SV1 (SEQ ID NOS:2 and 4) or fragments, variants, or fusion proteins thereof. In other embodiments, the antibodies of the invention preferentially bind to TR2 and TR2-SV1 (SEQ ID NOS:2 and 4), or fragments and variants thereof relative to their ability to bind TR2-SV2 (SEQ ID NO:6) or fragments, variants, or fusion proteins thereof. In other embodiments, the antibodies of the invention preferentially bind to TR2 and TR2-SV2 (SEQ ID NOS:2 and 6), or fragments and variants thereof relative to their ability to bind TR2-SV1 (SEQ ID NO:4) or fragments, variants, or fusion proteins thereof. In other embodiments, the antibodies of the invention preferentially bind to TR2-SV1 and TR2-SV2 (SEQ ID NOS:4 and 6), or fragments and variants thereof relative to their ability to bind TR2 (SEQ ID NO:2) or fragments, variants, or fusion proteins thereof. In other embodiments, the antibodies of the invention bind TR2, TR2-SV1, and TR2-SV2 (SEQ ID NOS:2, 4, and 6).

In another preferred embodiment, the antibodies of the present ivention bind to allelic variants of TR2 proteins. Nucleotide sequence variants which have been identified include either guanine or adenine at nucleotide 314 and either thymine or cytosine at nucleotides 386, 624 and 627 in SEQ ID NO:1. An antibody's ability to preferentially bind one antigen compared to another antigen may be determined using any method known in the art.

TR2 Polypeptides

TR2

In certain embodiments of the present invention, the antibodies of the present invention bind TR2 polypeptide, or fragments or variants thereof. The following section describes the TR2 polypeptides, fragments and variants that antibodies of the invention may bind in more detail. TR2 polypeptides which the antibodies of the invention may bind are described inmore detail in International Publication Numbers WO96/34095, WO98/18824, and WO00/56405, each of which are herein incorporated by reference in their entireties.

In certain embodiments, the antibodies of the present invention specifically bind TR2. An antibody that specifically binds TR2 may, in some embodiments, bind fragments, variants (including species orthologs and allelic variants of TR2), multimers or modified forms of TR2. For example, an antibody specific for TR2 may bind the TR2 moiety of a fusion protein comprising all or a portion of TR2.

In one embodiment, the antibodies of the invention preferentially bind TR2 (SEQ ID NO:2), or fragments, variants, or fusion proteins thereof (e.g., the extracellular region of TR2 fused to an Fc domain) relative to their ability to bind TR2-SV1 or TR2-SV2 (SEQ ID NOS:4 and 6; International Publication NOS: WO96/34095 and WO98/18824, which are herein incorporated by reference in their entirety) or fragments, variants, or fusion proteins thereof.

In another embodiment, antibodies of the invention preferentially bind TR2-SV1 (SEQ ID NO:4), fragments, variants, or fusion proteins thereof relative to their ability to bind TR2, or TR2-SV2 (SEQ ID NOS:2 and 6) or fragments, variants, or fusion proteins thereof. In another embodiment, antibodies of the invention preferentially bind TR2-SV2, fragments, variants, or fusion proteins thereof relative to their ability to bind TR2, or TR2-SV1 (SEQ ID NOS:2 and 4) or fragments, variants, or fusion proteins thereof. In other embodiments, the antibodies of the invention preferentially bind to TR2 and TR2-SV1 (SEQ ID NOS:2 and 4), or fragments and variants thereof relative to their ability to bind TR2-SV2 (SEQ ID NO:6) or fragments, variants, or fusion proteins thereof. In other embodiments, the antibodies of the invention preferentially bind to TR2 and TR2-SV2 (SEQ ID NOS:2 and 6), or fragments and variants thereof relative to their ability to bind TR2-SV1 (SEQ ID NO:4) or fragments, variants, or fusion proteins thereof. In other embodiments, the antibodies of the invention preferentially bind to TR2-SV1 and TR2-SV2 (SEQ ID NOS:4 and 6), or fragments and variants thereof relative to their ability to bind TR2 (SEQ ID NO:2) or fragments, variants, or fusion proteins thereof. In other embodiments, the antibodies of the invention bind TR2, TR2-SV1, and TR2-SV2 (SEQ ID NOS:2, 4, and 6).

TR2 proteins may be found as monomers or multimers (i.e., dimers, trimers, tetramers, and higher multimers). Accordingly, the present invention relates to antibodies that bind TR2 proteins found as monomers or as part of multimers. In specific embodiments, antibodies of the invention bind TR2 monomers, dimers, trimers or tetramers. In additional embodiments, antibodies of the invention bind at least dimers, at least trimers, or at least tetramers containing one or more TR2 polypeptides.

Antibodies of the invention may bind TR2 homomers or heteromers. As used herein, the term homomer, refers to a multimer containing only TR2 proteins of the invention (including TR2 fragments, variants, and fusion proteins, as described herein). These homomers may contain TR2 proteins having identical or different polypeptide sequences. In a specific embodiment, a homomer of the invention is a multimer containing only TR2 proteins having an identical polypeptide sequence. In another specific embodiment, antibodies of the invention bind TR2 homomers containing TR2 proteins having different polypeptide sequences. In specific embodiments, antibodies of the invention bind a TR2 homodimer (e.g., containing TR2 proteins having identical or different polypeptide sequences) or a homotrimer (e.g., containing TR2 proteins having identical or different polypeptide sequences). In additional embodiments, antibodies of the invention bind at least a homodimer, at least a homotrimer, or at least a homotetramer of TR2.

As used herein, the term heteromer refers to a multimer containing heterologous proteins (i.e., proteins containing polypeptide sequences that do not correspond to a polypeptide sequences encoded by the TR2 gene) in addition to the TR2 proteins of the invention. In a specific embodiment, antibodies of the invention bind a heterodimer, a heterotrimer, or a heterotetramer. In additional embodiments, the antibodies of the invention bind at least a heterodimer, at least a heterotrimer, or at least a heterotetramer containing one or more TR2 polypeptides.

Antibodies of the invention may bind TR2 polypeptide multimers that are the result of hydrophobic, hydrophilic, ionic and/or covalent associations and/or may be indirectly linked, by for example, liposome formation. Thus, in one embodiment, antibodies of the invention may bind multimers, such as, for example, homodimers or homotrimers, that are formed when TR2 proteins contact one another in solution. In another embodiment, antibodies of the invention may bind heteromultimers, such as, for example, heterotrimers or heterotetramers, that are formed when proteins of the invention contact antibodies to the TR2 polypeptides (including antibodies to the heterologous polypeptide sequence in a fusion protein) in solution. In other embodiments, multimers that one or more antibodies of the invention may bind are formed by covalent associations with and/or between the TR2 proteins of the invention. Such covalent associations may involve one or more amino acid residues contained in the polypeptide sequence of the protein (e.g., the polypeptide sequence recited in SEQ ID NO:2 or the polypeptide encoded by the deposited cDNA clone of ATCC Deposit 97059). In one instance, the covalent associations are cross-linking between cysteine residues located within the polypeptide sequences of the proteins which interact in the native (i.e., naturally occurring) polypeptide. In another instance, the covalent associations are the consequence of chemical or recombinant manipulation. Alternatively, such covalent associations may involve one or more amino acid residues contained in the heterologous polypeptide sequence in a TR2 fusion protein. In one example, covalent associations are between the heterologous sequence contained in a fusion protein (see, e.g., U.S. Pat. No. 5,478,925). In a specific example, the covalent associations are between the heterologous sequence contained in a TR2-Fc fusion protein (as described herein). In another specific example, covalent associations of fusion proteins are between heterologous polypeptide sequences from another TNF family ligand/receptor member that is capable of forming covalently associated multimers, such as for example, oseteoprotegerin (see, e.g., International Publication No. WO 98/49305, the contents of which are herein incorporated by reference in its entirety).

Antibodies of the invention may bind TR2 multimers that were generated using chemical techniques known in the art. For example, proteins desired to be contained in the multimers of the invention may be chemically cross-linked using linker molecules and linker molecule length optimization techniques known in the art (see, e.g., U.S. Pat. No. 5,478,925, which is herein incorporated by reference in its entirety). Additionally, multimers that may be bound by one or more antibodies of the invention may be generated using techniques known in the art to form one or more inter-molecule cross-links between the cysteine residues located within the polypeptide sequence of the proteins desired to be contained in the multimer (see, e.g., U.S. Pat. No. 5,478,925, which is herein incorporated by reference in its entirety). Further, proteins that may be bound by one or more antibodies of the invention may be routinely modified by the addition of cysteine or biotin to the C terminus or N-terminus of the polypeptide sequence of the protein and techniques known in the art may be applied to generate multimers containing one or more of these modified proteins (see, e.g., U.S. Pat. No. 5,478,925, which is herein incorporated by reference in its entirety). Additionally, techniques known in the art may be applied to generate liposomes containing the protein components desired to be contained in the multimer that one or more antibodies of the invention may bind(see, e.g., U.S. Pat. No. 5,478,925, which is herein incorporated by reference in its entirety).

Alternatively, antibodies of the invention may bind TR2 multimers that were generated using genetic engineering techniques known in the art. In one embodiment, proteins contained in multimers that may be bound by one or more antibodies of the invention are produced recombinantly using fusion protein technology described herein or otherwise known in the art (see, e.g., U.S. Pat. No. 5,478,925, which is herein incorporated by reference in its entirety). In a specific embodiment, polynucleotides coding for a homodimer that may be bound by one or more antibodies of the invention are generated by ligating a polynucleotide sequence encoding a TR2 polypeptide to a sequence encoding a linker polypeptide and then further to a synthetic polynucleotide encoding the translated product of the polypeptide in the reverse orientation from the original C-terminus to the N-terminus (lacking the leader sequence) (see, e.g., U.S. Pat. No. 5,478,925, which is herein incorporated by reference in its entirety). In another embodiment, recombinant techniques described herein or otherwise known in the art are applied to generate recombinant TR2 polypeptides which contain a transmembrane domain and which can be incorporated by membrane reconstitution techniques into liposomes (see, e.g., U.S. Pat. No. 5,478,925, which is herein incorporated by reference in its entirety). In another embodiment, two or more TR2 polypeptides are joined through synthetic linkers (e.g., peptide, carbohydrate or soluble polymer linkers). Examples include those peptide linkers described in U.S. Pat. No. 5,073,627 (hereby incorporated by reference). Proteins comprising multiple TR2 polypeptides separated by peptide linkers may be produced using conventional recombinant DNA technology. In specific embodiments, antibodies of the invention bind proteins comprising multiple TR2 polypeptides separated by peptide linkers.

Another method for preparing multimer TR2 polypeptides involves use of TR2 polypeptides fused to a leucine zipper or isoleucine polypeptide sequence. Leucine zipper domains and isoleucine zipper domains are polypeptides that promote multimerization of the proteins in which they are found. Leucine zippers were originally identified in several DNA-binding proteins (Landschulz et al., Science 240:1759, (1988)), and have since been found in a variety of different proteins. Among the known leucine zippers are naturally occurring peptides and derivatives thereof that dimerize or trimerize. Examples of leucine zipper domains suitable for producing soluble multimeric TR2 proteins are those described in PCT application WO 94/10308, hereby incorporated by reference. Recombinant fusion proteins comprising a soluble TR2 polypeptide fused to a peptide that dimerizes or trimerizes in solution are expressed in suitable host cells, and the resulting soluble multimeric TR2 is recovered from the culture supernatant using techniques known in the art. In specific embodiments, antibodies of the invention bind the TR2 moiety of TR2-leucine zipper fusion protein monomers and/or TR2-leucine zipper fusion protein multimers.

Certain members of the TNF family of proteins are believed to exist in trimeric form (Beutler and Huffel, Science 264:667, 1994; Banner et al., Cell 73:431, 1993). Thus, trimeric TR2 may offer the advantage of enhanced biological activity. Preferred leucine zipper moieties are those that preferentially form trimers. One example is a leucine zipper derived from lung surfactant protein D (SPD), as described in Hoppe et al. (FEBS Letters 344:191, (1994)) and in U.S. patent application Ser. No. 08/446,922, hereby incorporated by reference. In specific embodiments, antibodies of the invention bind the TR2 moiety of TR2-leucine zipper fusion protein trimers.

Other peptides derived from naturally occurring trimeric proteins may be employed in preparing trimeric TR2. In specific embodiments, antibodies of the invention bind the TR2 moiety of TR2-fusion protein monomers and/or TR2 fusion protein trimers.

Antibodies that bind TR2 polypeptides may bind them as isolated polypeptides or in their naturally occurring state. For, example antibodies of the present invention may bind recombinantly produced TR2 polypeptides. In a specific embodiment, antibodies of the present invention bind a TR2 polypeptide expressed on the surface of a cell wherein the TR2 protein is encoded by a polynucleotide encoding amino acids 1 to 283 of SEQ ID NO:2 operably associated with a regulatory sequence that controls expression of said polynucleotide.

Antibodies of the present invention may bind TR2 polypeptide fragments comprising or alternatively, consisting of, an amino acid sequence contained in SEQ ID NO:2, encoded by the cDNA contained in ATCC deposit Number 97059, or encoded by nucleic acids which hybridize (e.g., under stringent hybridization conditions) to the nucleotide sequence contained in ATCC deposit Number 97059, or the complementary strand thereto. Protein fragments may be “free-standing,” or comprised within a larger polypeptide of which the fragment forms a part or region, most preferably as a single continuous region. Antibodies of the present invention may bind polypeptide fragments, including, for example, fragments that comprise or alternatively, consist of from about amino acid residues: 1 to 23, 24 to 43, 44 to 63, 64 to 83, 84 to 103, 104 to 123, 124 to 143, 144 to 163, 164 to 183, 184 to 203, 204 to 225, 226 to 238, 239 to 264, and/or 265 to 283 of SEQ ID NO:2. In this context “about” includes the particularly recited value, larger or smaller by several (5, 4, 3, 2, or 1) amino acids, at either extreme or at both extremes. Moreover, polypeptide fragments that the antibodies of the invention may bind can be at least about 10, 20, 30, 40, 50, 60, 70, 80, 90, 100, 110, 120, 130, 140, 150, 175 or 200 amino acids in length. In this context “about” includes the particularly recited value, larger or smaller by several (5, 4, 3, 2, or 1) amino acids, at either extreme or at both extremes.

Preferably, antibodies of the present invention bind polypeptide fragments selected from the group: a polypeptide comprising or alternatively, consisting of, the TR2 extracellular domain (predicted to constitute amino acid residues from about 37 to about 200 in SEQ ID NO:2); a polypeptide comprising or alternatively, consisting of, a TR2 cysteine rich domains (which may be found in the protein fragment consisting of amino acid residues from about 42 to about 184 in SEQ ID NO:2); a polypeptide comprising or alternatively, consisting of, the TR2 transmembrane domain (predicted to constitute amino acid residues from about 201 to about 225 in SEQ ID NO:2); a polypeptide comprising or alternatively, consisting of, fragment of the predicted mature TR2 polypeptide, wherein the fragment has a TR2 functional activity (e.g., antigenic activity or biological acitivity); a polypeptide comprising or alternatively, consisting of, the TR2 intracellular domain (predicted to constitute amino acid residues from about 226 to about 283 in SEQ ID NO:2); and a polypeptide comprising, or alternatively, consisting of, one, two, three, four or more, epitope bearing portions of the TR2 protein. In additional embodiments, the polypeptide fragments of the invention comprise, or alternatively, consist of, any combination of 1, 2, 3, 4, or all 5 of the above members. The amino acid residues constituting the TR2 extracellular, transmembrane and intracellular domains have been predicted by computer analysis. Thus, as one of ordinary skill would appreciate, the amino acid residues constituting these domains may vary slightly (e.g., by about 1 to about 15 amino acid residues) depending on the criteria used to define each domain. Polynucleotides encoding these polypeptides are also encompassed by the invention.

It is believed that one or both of the extracellular cysteine rich motifs of TR2 is important for interactions between TR2 and its ligands (e.g., LIGHT or LTα). Accordingly, in highly preferred embodiments, antibodies of the present invention bind TR2 polypeptide fragments comprising, or alternatively consisting of amino acid residues 42 to 185 of SEQ ID NO:2. In another preferred embodiment, antibodies of the present invention bind TR2 polypeptides comprising, or alternatively consisting the extracellular soluble domain of TR2 (amino acid residues 36-200 of SEQ ID NO:2) In highly preferred embodiments, the antibodies of the invention that bind all or a portion of the extracellular soluble domain of TR2 prevent TR2 ligand (e.g. TL5 or LTα) from binding to TR2. In other highly preferred embodiments, the antibodies of the invention that bind all or a portion of the extracellular soluble domain of TR2 antagonize TR2. In other highly preferred embodiments, the antibodies of the invention that bind all or a portion of the extracellular soluble domain of TR2 inhibit proliferation of the cells expressing TR2. In other highly preferred embodiments, the antibodies of the invention that bind all or a portion of the extracellular soluble domain of TR2 inhibit differentiation of the cells expressing TR2 (e.g. T-cells). In other highly preferred embodiments, the antibodies of the invention that bind all or a portion of the extracellular soluble domain of TR2 inhibit HSV infection of cells expressing the TR2.

Antibodies of the invention may also bind fragments comprising, or alternatively, consisting of structural or functional attributes of TR2. Such fragments include amino acid residues that comprise alpha-helix and alpha-helix forming regions (“alpha-regions”), beta-sheet and beta-sheet-forming regions (“beta-regions”), turn and turn-forming regions (“turn-regions”), coil and coil-forming regions (“coil-regions”), hydrophilic regions, hydrophobic regions, alpha amphipathic regions, beta amphipathic regions, surface forming regions, and high antigenic index regions (i.e., containing four or more contiguous amino acids having an antigenic index of greater than or equal to 1.5, as identified using the default parameters of the Jameson-Wolf program) of complete (i.e., full-length) TR2. Certain preferred regions are those set out in Table 2 and include, but are not limited to, regions of the aforementioned types identified by analysis of the amino acid sequence depicted in (SEQ ID NO:2), such preferred regions include; Garnier-Robson predicted alpha-regions, beta-regions, turn-regions, and coil-regions; Chou-Fasman predicted alpha-regions, beta-regions, and turn-regions; Kyte-Doolittle predicted hydrophilic regions; Eisenberg alpha and beta amphipathic regions; Emini surface-forming regions; and Jameson-Wolf high antigenic index regions, as predicted using the default parameters of these computer programs.

The data representing the structural or functional attributes of TR2 set forth in Table 2, as described above, was generated using the various modules and algorithms of the DNA*STAR set on default parameters. Column I represents the results of a Garnier-Robson analysis of alpha helical regions; Column II represents the results of a Chou-Fasman analysis of alpha helical regions; Column III represents the results of a Garnier Robson analysis of beta sheet regions; Column IV represents the results of a Chou-Fasman analysis of beta sheet regions; Column V represents the results of a Garnier Robson analysis of turn regions; Column VI represents the results of a Chou-Fasman analysis of turn regions; Column VII represents the results of a Garnier Robson analysis of coil regions; Column VIII represents a Kyte-Doolittle hydrophilicity plot; Column; Column IX represents a Hopp-Woods hydrophobicity plot; Column X represents the results of an Eisenberg analysis of alpha amphipathic regions; Column XI represents the results of an Eisenberg analysis of beta amphipathic regions; Column XII represents the results of a Karplus-Schultz analysis of flexible regions; Column XIII represents the Jameson-Wolf antigenic index score; and Column XIV represents the Emini surface probability plot.

In a preferred embodiment, the data presented in columns VIII, XIII, and XIV of Table 2 can be used to determine regions of TR2 which exhibit a high degree of potential for antigenicity. Regions of high antigenicity are determined from the data presented in columns VIII, XIII, and/or XIV by choosing values which represent regions of the polypeptide which are likely to be exposed on the surface of the polypeptide in an environment in which antigen recognition may occur in the process of initiation of an immune response.

The above-mentioned preferred regions set out in Table 2 include, but are not limited to, regions of the aforementioned types identified by analysis of the amino acid sequence set out in SEQ ID NO:2. As set out in Table 2, such preferred regions include Garnier-Robson alpha-regions, beta-regions, turn-regions, and coil-regions, Chou-Fasman alpha-regions, beta-regions, and turn-regions, Kyte-Doolittle hydrophilic regions, Eisenberg alpha- and beta-amphipathic regions, Karplus-Schulz flexible regions, Jameson-Wolf regions of high antigenic index and Emini surface-forming regions. Among preferred polypeptide fragments that one or more antibodies of the invention may bind are those that comprise regions of TR2 that combine several structural features, such as several (e.g., 1, 2, 3 , or 4) of the same or different region features set out above and in Table 2.

TABLE II

| Res | Position | I | II | III | IV | V | VI | VII | VIII | IX | X | XI | XII | XIII | XIV |
|---|---|---|---|---|---|---|---|---|---|---|---|---|---|---|---|
| Met | 1 | . | . | B | . | . | . | . | 0.58 | −0.24 | . | . | . | 1.15 | 1.53 |
| Glu | 2 | . | . | B | . | . | . | . | 0.97 | −0.24 | . | * | . | 1.40 | 1.19 |
| Pro | 3 | . | . | . | . | . | T | C | 1.07 | −0.67 | * | * | . | 2.35 | 1.55 |
| Pro | 4 | . | . | . | . | T | T | . | 1.11 | −0.19 | * | * | . | 2.50 | 1.65 |
| Gly | 5 | . | . | . | . | T | T | . | 1.29 | −0.37 | . | * | F | 2.25 | 0.94 |
| Asp | 6 | . | . | . | . | T | T | . | 1.68 | 0.06 | * | * | F | 1.40 | 0.94 |
| Trp | 7 | . | . | . | . | T | . | . | 1.47 | 0.06 | . | . | F | 0.95 | 0.94 |
| Gly | 8 | . | . | . | . | . | . | C | 1.39 | 0.06 | * | * | F | 0.65 | 1.47 |
| Pro | 9 | . | . | . | . | . | T | C | 1.71 | 0.54 | * | * | F | 0.15 | 0.93 |
| Pro | 10 | . | . | . | . | . | T | C | 1.76 | 0.54 | * | * | F | 0.30 | 1.73 |
| Pro | 11 | . | . | . | . | T | T | . | 1.44 | 0.01 | * | * | F | 0.80 | 2.34 |
| Trp | 12 | . | . | . | . | T | T | . | 1.52 | 0.07 | * | * | F | 0.80 | 2.18 |
| Arg | 13 | . | . | . | . | T | . | . | 1.91 | 0.07 | * | * | F | 0.94 | 2.18 |
| Ser | 14 | . | . | . | . | . | . | C | 1.81 | −0.36 | * | * | F | 1.68 | 2.82 |
| Thr | 15 | . | . | . | . | . | T | C | 2.02 | −0.30 | * | * | F | 2.22 | 3.88 |
| Pro | 16 | . | . | . | . | . | T | C | 1.38 | −1.21 | . | * | F | 2.86 | 3.30 |
| Lys | 17 | . | . | . | . | T | T | . | 0.86 | −0.57 | . | * | F | 3.40 | 1.83 |
| Thr | 18 | . | . | B | . | . | T | . | 0.86 | −0.27 | . | * | F | 2.36 | 1.05 |
| Asp | 19 | . | . | B | B | . | . | . | 0.34 | −0.76 | * | . | F | 1.92 | 1.32 |
| Val | 20 | . | . | B | B | . | . | . | −0.20 | −0.50 | * | * | . | 1.28 | 0.55 |
| Leu | 21 | . | . | B | B | . | . | . | −0.80 | 0.14 | * | . | . | 0.04 | 0.28 |
| Arg | 22 | . | . | B | B | . | . | . | −1.09 | 0.34 | * | * | . | −0.30 | 0.14 |
| Leu | 23 | . | . | B | B | . | . | . | −1.59 | 1.10 | * | * | . | −0.60 | 0.29 |
| Val | 24 | . | . | B | B | . | . | . | −1.90 | 1.14 | * | * | . | −0.60 | 0.29 |
| Leu | 25 | . | . | B | B | . | . | . | −1.74 | 0.94 | * | * | . | −0.60 | 0.22 |
| Tyr | 26 | . | . | B | B | . | . | . | −1.74 | 1.73 | * | * | . | −0.60 | 0.23 |
| Leu | 27 | . | . | B | B | . | . | . | −2.20 | 1.73 | * | * | . | −0.60 | 0.25 |
| Thr | 28 | . | . | B | B | . | . | . | −1.98 | 1.51 | . | . | . | −0.60 | 0.30 |
| Phe | 29 | . | . | B | B | . | . | . | −1.33 | 1.33 | . | . | . | −0.60 | 0.19 |
| Leu | 30 | . | . | B | B | . | . | . | −1.19 | 1.00 | . | . | . | −0.60 | 0.37 |
| Gly | 31 | . | . | B | B | . | . | . | −1.19 | 0.89 | . | . | . | −0.60 | 0.14 |
| Ala | 32 | . | . | B | . | . | T | . | −0.97 | 1.16 | . | . | . | −0.20 | 0.25 |
| Pro | 33 | . | . | . | . | T | T | . | −0.87 | 0.87 | . | . | . | 0.20 | 0.30 |
| Cys | 34 | . | . | . | . | T | T | . | −0.76 | 0.61 | . | . | . | 0.20 | 0.47 |
| Tyr | 35 | . | . | B | . | . | T | . | −0.76 | 0.69 | . | . | . | −0.20 | 0.47 |
| Ala | 36 | . | . | B | . | . | . | . | −0.62 | 0.87 | . | . | . | −0.40 | 0.25 |
| Pro | 37 | . | . | B | . | . | . | . | −0.33 | 0.87 | . | . | . | −0.40 | 0.72 |
| Ala | 38 | . | . | B | . | . | . | . | −0.79 | 0.69 | . | . | . | −0.14 | 0.62 |
| Leu | 39 | . | . | B | . | . | T | . | −0.08 | 0.50 | . | . | . | 0.32 | 0.33 |
| Pro | 40 | . | . | B | . | . | T | . | 0.17 | 0.00 | . | . | F | 1.03 | 0.42 |
| Ser | 41 | . | . | . | . | T | T | . | 0.76 | −0.43 | . | . | F | 2.29 | 0.73 |
| Cys | 42 | . | . | B | . | . | T | . | 0.97 | −0.93 | . | . | F | 2.60 | 1.47 |
| Lys | 43 | . | A | B | . | . | . | . | 1.31 | −1.61 | . | . | F | 1.94 | 1.65 |
| Glu | 44 | . | A | B | . | . | . | . | 1.91 | −1.29 | . | . | F | 1.68 | 1.93 |
| Asp | 45 | . | A | . | . | T | . | . | 1.27 | −1.24 | . | . | F | 1.82 | 5.56 |
| Glu | 46 | . | A | B | . | . | . | . | 1.22 | −1.17 | . | . | F | 1.16 | 2.06 |
| Tyr | 47 | . | A | B | . | . | . | . | 1.59 | −0.74 | . | . | . | 0.75 | 1.18 |
| Pro | 48 | . | . | . | . | T | . | . | 1.54 | −0.36 | . | . | . | 0.90 | 0.95 |
| Val | 49 | . | . | . | . | T | . | . | 0.88 | −0.36 | . | . | F | 1.05 | 0.95 |
| Gly | 50 | . | . | . | . | T | T | . | 0.21 | 0.21 | . | . | F | 0.65 | 0.32 |
| Ser | 51 | . | . | . | . | T | T | . | 0.00 | 0.03 | . | * | F | 0.65 | 0.11 |
| Glu | 52 | . | . | . | . | T | T | . | 0.29 | 0.03 | . | * | F | 0.90 | 0.23 |
| Cys | 53 | . | . | B | . | . | T | . | −0.17 | −0.61 | . | * | . | 1.50 | 0.47 |
| Cys | 54 | . | . | B | . | . | T | . | 0.39 | −0.47 | . | * | . | 1.45 | 0.19 |
| Pro | 55 | . | . | . | . | T | T | . | 0.52 | −0.47 | . | . | . | 2.10 | 0.15 |
| Lys | 56 | . | . | . | . | T | T | . | 0.48 | −0.04 | . | . | F | 2.50 | 0.42 |
| Cys | 57 | . | . | . | . | T | T | . | 0.23 | −0.19 | * | * | F | 2.25 | 0.78 |
| Ser | 58 | . | . | . | . | . | T | C | 1.01 | 0.00 | * | * | F | 1.20 | 0.79 |
| Pro | 59 | . | . | . | . | T | T | . | 0.82 | −0.43 | . | * | F | 1.75 | 0.77 |
| Gly | 60 | . | . | . | . | T | T | . | 1.08 | 0.21 | . | * | F | 1.05 | 1.07 |
| Tyr | 61 | . | . | B | . | . | T | . | 1.03 | −0.36 | . | * | . | 0.85 | 1.60 |
| Arg | 62 | . | A | B | . | . | . | . | 1.11 | −0.74 | * | * | . | 0.75 | 1.79 |
| Val | 63 | . | A | B | . | . | . | . | 0.74 | −0.67 | * | * | . | 0.75 | 1.83 |
| Lys | 64 | . | A | B | . | . | . | . | 0.61 | −0.53 | * | * | . | 0.60 | 0.63 |
| Glu | 65 | . | A | B | . | . | . | . | 0.96 | −0.86 | * | * | . | 0.60 | 0.32 |
| Ala | 66 | . | A | B | . | . | . | . | 0.39 | −0.86 | * | * | . | 0.60 | 0.74 |
| Cys | 67 | . | A | B | . | . | . | . | −0.03 | −0.81 | * | * | . | 0.60 | 0.30 |
| Gly | 68 | . | A | . | . | T | . | . | 0.48 | −0.33 | * | * | . | 0.70 | 0.25 |
| Glu | 69 | . | A | . | . | T | . | . | 0.12 | 0.10 | * | * | F | 0.25 | 0.25 |
| Leu | 70 | . | . | . | B | T | . | . | −0.73 | 0.09 | * | * | F | 0.25 | 0.67 |
| Thr | 71 | . | . | . | B | T | . | . | −0.81 | 0.16 | * | * | F | 0.25 | 0.50 |
| Gly | 72 | . | . | . | B | T | . | . | −0.14 | 0.30 | * | * | F | 0.25 | 0.16 |
| Thr | 73 | . | . | . | B | T | . | . | −0.01 | 0.30 | * | * | F | 0.25 | 0.33 |
| Val | 74 | . | . | B | B | . | . | . | −0.68 | 0.04 | * | * | . | −0.30 | 0.35 |
| Cys | 75 | . | . | B | B | . | . | . | −0.08 | 0.13 | . | . | . | −0.30 | 0.19 |
| Glu | 76 | . | . | B | B | . | . | . | 0.02 | 0.13 | . | . | . | −0.23 | 0.20 |
| Pro | 77 | . | . | . | . | T | . | . | 0.02 | 0.07 | . | . | F | 0.59 | 0.42 |
| Cys | 78 | . | . | . | . | . | . | C | 0.02 | −0.14 | . | . | F | 1.06 | 0.78 |
| Pro | 79 | . | . | . | . | . | T | C | 0.63 | −0.23 | . | . | F | 1.33 | 0.65 |
| Pro | 80 | . | . | . | . | T | T | . | 0.41 | 0.53 | . | . | F | 0.70 | 0.66 |
| Gly | 81 | . | . | . | . | T | T | . | −0.18 | 0.79 | . | . | F | 0.63 | 0.86 |
| Thr | 82 | . | . | B | . | . | T | . | 0.00 | 0.71 | . | . | F | 0.16 | 0.56 |
| Tyr | 83 | . | . | B | . | . | . | . | −0.14 | 0.79 | . | . | . | −0.26 | 0.49 |
| Ile | 84 | . | . | B | . | . | . | . | 0.07 | 1.04 | . | . | . | −0.33 | 0.41 |
| Ala | 85 | . | . | B | . | . | . | . | −0.07 | 1.01 | . | . | . | −0.40 | 0.46 |
| His | 86 | . | . | B | . | . | T | . | −0.53 | 0.96 | . | . | . | −0.20 | 0.29 |
| Leu | 87 | . | . | B | . | . | T | . | −0.52 | 0.89 | * | . | . | −0.20 | 0.34 |
| Asn | 88 | . | . | . | . | T | T | . | −0.23 | 0.59 | * | * | . | 0.20 | 0.45 |
| Gly | 89 | . | . | . | . | T | T | . | −0.01 | 0.09 | * | . | F | 0.65 | 0.66 |
| Leu | 90 | . | . | . | . | T | . | . | −0.23 | 0.16 | * | * | F | 0.45 | 0.43 |
| Ser | 91 | . | . | . | . | T | T | . | −0.20 | 0.16 | * | . | F | 0.65 | 0.22 |
| Lys | 92 | . | . | . | . | T | T | . | −0.06 | 0.16 | * | * | . | 0.50 | 0.39 |
| Cys | 93 | . | . | B | . | . | T | . | −0.06 | 0.30 | * | * | . | 0.10 | 0.25 |
| Leu | 94 | . | . | B | . | . | T | . | −0.31 | 0.01 | * | . | . | 0.10 | 0.33 |
| Gln | 95 | . | A | B | . | . | . | . | −0.17 | 0.24 | * | . | . | −0.30 | 0.16 |
| Cys | 96 | . | A | B | . | . | . | . | 0.13 | 0.81 | * | . | . | −0.60 | 0.16 |
| Gln | 97 | . | A | B | . | . | . | . | −0.12 | 0.24 | * | . | . | −0.30 | 0.33 |
| Met | 98 | . | A | B | . | . | . | . | −0.04 | −0.01 | . | . | . | 0.30 | 0.29 |
| Cys | 99 | . | A | B | . | . | . | . | 0.17 | 0.09 | . | * | . | −0.30 | 0.55 |
| Asp | 100 | . | . | B | . | . | . | . | −0.18 | 0.13 | . | . | . | −0.10 | 0.31 |
| Pro | 101 | . | . | B | . | . | . | . | −0.32 | 0.16 | . | * | . | −0.10 | 0.31 |
| Ala | 102 | . | A | B | . | . | . | . | −0.21 | 0.23 | . | * | . | −0.30 | 0.48 |
| Met | 103 | . | A | B | . | . | . | . | −0.20 | −0.34 | . | * | . | 0.30 | 0.57 |
| Gly | 104 | . | A | B | . | . | . | . | 0.17 | 0.16 | * | * | . | −0.30 | 0.37 |
| Leu | 105 | . | A | B | . | . | . | . | 0.28 | 0.11 | * | * | . | −0.30 | 0.49 |
| Arg | 106 | . | A | B | . | . | . | . | 0.49 | −0.39 | * | * | . | 0.64 | 0.97 |
| Ala | 107 | . | A | B | . | . | . | . | 0.41 | −0.60 | * | * | . | 1.43 | 1.58 |
| Ser | 108 | . | . | . | . | T | T | . | 0.71 | −0.46 | * | * | F | 2.42 | 1.03 |
| Arg | 109 | . | . | . | . | T | T | . | 1.17 | −0.76 | * | * | F | 2.91 | 0.70 |
| Asn | 110 | . | . | . | . | T | T | . | 1.67 | −0.76 | * | * | F | 3.40 | 1.36 |
| Cys | 111 | . | . | . | . | T | T | . | 1.56 | −0.77 | * | * | F | 3.06 | 1.47 |
| Ser | 112 | . | . | . | . | T | . | . | 2.14 | −1.16 | * | . | F | 2.52 | 1.30 |
| Arg | 113 | . | A | . | . | T | . | . | 1.86 | −0.76 | * | * | F | 1.98 | 1.30 |
| Thr | 114 | . | A | . | . | T | . | . | 0.89 | −0.66 | * | * | F | 1.64 | 2.44 |
| Glu | 115 | . | A | . | . | T | . | . | 0.22 | −0.59 | * | . | F | 1.30 | 1.35 |
| Asn | 116 | . | A | B | . | . | . | . | 0.54 | −0.40 | * | . | F | 0.45 | 0.37 |
| Ala | 117 | . | A | B | . | . | . | . | 0.18 | 0.03 | * | . | . | −0.30 | 0.25 |
| Val | 118 | . | A | . | . | T | . | . | −0.23 | 0.11 | * | * | . | 0.10 | 0.08 |
| Cys | 119 | . | . | . | . | T | . | . | −0.13 | 0.50 | . | . | . | 0.00 | 0.07 |
| Gly | 120 | . | . | . | . | T | . | . | −0.48 | 0.53 | . | . | . | 0.00 | 0.10 |
| Cys | 121 | . | . | . | . | T | . | . | −0.51 | 0.46 | . | . | . | 0.00 | 0.13 |
| Ser | 122 | . | . | . | . | . | T | C | −0.62 | 0.31 | . | . | . | 0.30 | 0.34 |
| Pro | 123 | . | . | . | . | T | T | . | −0.43 | 0.53 | . | * | F | 0.35 | 0.30 |
| Gly | 124 | . | . | . | . | T | T | . | −0.66 | 0.67 | . | . | . | 0.20 | 0.30 |
| His | 125 | . | . | B | . | . | T | . | −1.17 | 0.79 | . | . | . | −0.20 | 0.16 |
| Phe | 126 | . | . | B | B | . | . | . | −0.50 | 1.04 | . | . | . | −0.60 | 0.07 |
| Cys | 127 | . | . | B | B | . | . | . | −0.20 | 1.01 | . | . | . | −0.32 | 0.13 |
| Ile | 128 | . | . | B | B | . | . | . | −0.33 | 0.59 | . | * | . | −0.04 | 0.16 |
| Val | 129 | . | . | B | . | . | T | . | 0.01 | 0.51 | . | * | . | 0.64 | 0.18 |
| Gln | 130 | . | . | . | . | T | T | . | 0.01 | −0.27 | . | . | F | 2.37 | 0.57 |
| Asp | 131 | . | . | . | . | T | T | . | 0.04 | −0.34 | * | . | F | 2.80 | 1.11 |
| Gly | 132 | . | . | . | . | T | T | . | 0.12 | −0.46 | * | . | F | 2.37 | 0.80 |
| Asp | 133 | . | A | . | . | T | . | . | 0.42 | −0.60 | * | . | F | 1.99 | 0.47 |
| His | 134 | . | A | . | . | T | . | . | 0.61 | −0.50 | . | * | . | 1.56 | 0.28 |
| Cys | 135 | . | A | B | . | . | . | . | 0.72 | 0.07 | . | * | . | −0.02 | 0.15 |
| Ala | 136 | . | A | B | . | . | . | . | 0.13 | −0.36 | . | * | . | 0.30 | 0.18 |
| Ala | 137 | . | A | B | . | . | . | . | 0.23 | 0.14 | . | * | . | −0.30 | 0.13 |
| Cys | 138 | . | A | B | . | . | . | . | −0.36 | 0.40 | . | * | . | −0.30 | 0.39 |
| Arg | 139 | . | A | B | . | . | . | . | −0.63 | 0.33 | . | * | . | −0.30 | 0.39 |
| Ala | 140 | . | A | B | . | . | . | . | −0.27 | 0.31 | . | * | . | −0.30 | 0.56 |
| Tyr | 141 | . | . | B | . | . | . | . | 0.02 | 0.20 | . | * | . | 0.05 | 1.39 |
| Ala | 142 | . | . | B | . | . | . | . | 0.40 | 0.01 | * | * | . | −0.10 | 0.95 |
| Thr | 143 | . | . | . | . | T | . | . | 0.72 | 0.44 | . | * | F | 0.30 | 1.45 |
| Ser | 144 | . | . | . | . | . | . | C | 0.61 | 0.37 | * | * | F | 0.25 | 0.92 |
| Ser | 145 | . | . | . | . | . | T | C | 1.31 | 0.01 | * | * | F | 0.60 | 1.57 |
| Pro | 146 | . | . | . | . | . | T | C | 0.70 | −0.49 | * | * | F | 1.20 | 2.14 |
| Gly | 147 | . | . | . | . | T | T | . | 1.29 | −0.33 | * | . | F | 1.40 | 1.18 |
| Gln | 148 | . | . | B | . | . | T | . | 1.64 | −0.31 | * | . | F | 1.00 | 1.53 |
| Arg | 149 | . | . | B | . | . | . | . | 1.60 | −0.70 | * | . | F | 1.40 | 1.98 |
| Val | 150 | . | . | B | . | . | . | . | 1.56 | −0.70 | * | . | F | 1.70 | 1.98 |
| Gln | 151 | . | . | B | . | . | T | . | 1.46 | −0.70 | * | . | F | 2.20 | 1.13 |
| Lys | 152 | . | . | B | . | . | T | . | 1.80 | −0.61 | * | . | F | 2.35 | 0.83 |
| Gly | 153 | . | . | . | . | . | T | C | 1.50 | −0.61 | * | . | F | 3.00 | 1.94 |
| Gly | 154 | . | . | . | . | . | T | C | 1.39 | −0.87 | * | * | F | 2.70 | 1.50 |
| Thr | 155 | . | . | . | . | . | . | C | 2.24 | −0.87 | * | . | F | 2.45 | 1.30 |
| Glu | 156 | . | . | . | . | . | . | C | 1.93 | −0.87 | * | . | F | 2.40 | 2.20 |
| Ser | 157 | . | . | B | . | T | T | . | 1.08 | −0.81 | * | . | F | 2.75 | 3.20 |
| Gln | 158 | . | . | . | . | T | T | . | 0.76 | −0.56 | * | . | F | 2.70 | 1.83 |
| Asp | 159 | . | . | . | . | T | T | . | 1.10 | −0.47 | * | . | F | 2.50 | 0.57 |
| Thr | 160 | . | . | B | . | . | T | . | 1.41 | −0.07 | * | . | F | 1.85 | 0.73 |
| Leu | 161 | . | . | . | . | T | . | . | 0.74 | −0.06 | * | . | . | 1.65 | 0.68 |
| Cys | 162 | . | . | . | . | T | T | . | 0.83 | 0.11 | . | . | . | 1.00 | 0.22 |
| Gln | 163 | . | . | . | . | T | T | . | 0.62 | 0.54 | . | . | . | 0.45 | 0.23 |
| Asn | 164 | . | . | . | . | T | T | . | 0.28 | 0.49 | * | . | . | 0.20 | 0.44 |
| Cys | 165 | . | . | B | . | . | T | . | 0.28 | 0.23 | . | . | F | 0.25 | 0.81 |
| Pro | 166 | . | . | . | . | . | T | C | 0.39 | 0.14 | . | . | F | 0.45 | 0.67 |
| Pro | 167 | . | . | . | . | T | T | . | 0.76 | 0.53 | . | . | F | 0.35 | 0.36 |
| Gly | 168 | . | . | . | . | T | T | . | 0.54 | 0.51 | . | . | F | 0.35 | 0.91 |
| Thr | 169 | . | . | . | . | T | T | . | 0.54 | 0.37 | . | * | F | 0.65 | 0.91 |
| Phe | 170 | . | . | B | . | . | . | . | 0.87 | 0.34 | . | * | F | 0.05 | 0.94 |
| Ser | 171 | . | . | . | . | . | T | C | 0.77 | 0.34 | . | * | F | 0.45 | 0.94 |
| Pro | 172 | . | . | . | . | . | T | C | 0.17 | 0.40 | . | * | F | 0.45 | 0.94 |
| Asn | 173 | . | . | . | . | T | T | . | 0.51 | 0.60 | . | * | F | 0.35 | 0.90 |
| Gly | 174 | . | . | . | . | . | T | C | 0.82 | −0.19 | . | * | F | 1.20 | 1.16 |
| Thr | 175 | . | A | . | . | . | . | C | 0.86 | −0.57 | . | * | F | 1.10 | 1.30 |
| Leu | 176 | . | A | . | . | . | . | C | 1.16 | −0.43 | . | . | F | 0.65 | 0.43 |
| Glu | 177 | . | A | B | . | . | . | . | 1.33 | −0.43 | . | . | F | 0.45 | 0.76 |
| Glu | 178 | . | A | B | . | . | . | . | 1.33 | −0.36 | . | . | . | 0.30 | 0.72 |
| Cys | 179 | . | A | B | . | . | . | . | 1.37 | −0.44 | . | * | . | 0.45 | 1.50 |
| Gln | 180 | . | A | . | . | T | . | . | 1.72 | −0.64 | * | * | . | 1.15 | 1.25 |
| His | 181 | . | A | . | . | T | . | . | 1.87 | −0.64 | . | * | . | 1.15 | 1.45 |
| Gln | 182 | . | A | . | . | T | . | . | 1.57 | −0.07 | . | * | F | 1.00 | 1.45 |
| Thr | 183 | . | . | . | . | T | T | . | 1.28 | −0.26 | . | * | F | 1.40 | 1.12 |
| Lys | 184 | . | . | . | . | T | T | . | 1.13 | 0.26 | . | * | F | 0.65 | 0.87 |
| Cys | 185 | . | . | . | . | T | T | . | 0.28 | 0.44 | . | * | . | 0.20 | 0.41 |
| Ser | 186 | . | . | . | . | T | T | . | −0.00 | 0.69 | . | . | . | 0.20 | 0.21 |
| Trp | 187 | . | . | B | B | . | . | . | 0.04 | 0.69 | . | * | . | −0.60 | 0.15 |
| Leu | 188 | . | . | B | B | . | . | . | −0.23 | 0.69 | * | . | . | −0.60 | 0.57 |
| Val | 189 | . | . | B | B | . | . | . | −0.62 | 0.61 | * | . | . | −0.60 | 0.43 |
| Thr | 190 | . | . | B | B | . | . | . | −0.54 | 0.66 | . | . | . | −0.39 | 0.40 |
| Lys | 191 | . | . | B | B | . | . | . | −0.59 | 0.24 | . | . | F | 0.27 | 0.50 |
| Ala | 192 | . | . | B | . | . | . | . | −0.61 | −0.01 | . | . | F | 1.28 | 0.66 |
| Gly | 193 | . | . | . | . | . | T | C | −0.10 | −0.17 | . | . | F | 1.89 | 0.66 |
| Ala | 194 | . | . | . | . | . | T | C | 0.46 | −0.27 | . | . | F | 2.10 | 0.44 |
| Gly | 195 | . | . | . | . | . | T | C | 0.47 | 0.11 | . | . | F | 1.29 | 0.59 |
| Thr | 196 | . | . | . | . | . | T | C | 0.39 | −0.00 | . | . | F | 1.68 | 0.80 |
| Ser | 197 | . | . | . | . | . | . | C | 0.69 | 0.07 | . | . | F | 0.82 | 1.07 |
| Ser | 198 | . | . | . | . | . | T | C | 0.18 | 0.49 | . | . | F | 0.51 | 1.14 |
| Ser | 199 | . | . | . | . | . | T | C | 0.48 | 0.70 | . | . | F | 0.15 | 0.59 |
| His | 200 | . | . | . | . | T | T | . | 0.53 | 1.13 | . | . | . | 0.20 | 0.46 |
| Trp | 201 | . | . | B | . | . | T | . | 0.14 | 1.66 | . | . | . | −0.20 | 0.36 |
| Val | 202 | . | . | B | B | . | . | . | −0.37 | 2.06 | . | . | . | −0.60 | 0.23 |
| Trp | 203 | . | . | B | B | . | . | . | −0.37 | 2.36 | . | . | . | −0.60 | 0.14 |
| Trp | 204 | . | . | B | B | . | . | . | −0.41 | 2.24 | . | . | . | −0.60 | 0.18 |
| Phe | 205 | . | . | B | B | . | . | . | −0.68 | 1.76 | . | . | . | −0.60 | 0.24 |
| Leu | 206 | . | . | . | . | . | T | C | −1.20 | 1.50 | . | . | . | 0.00 | 0.31 |
| Ser | 207 | . | . | . | . | . | T | C | −1.20 | 1.27 | . | * | F | 0.15 | 0.24 |
| Gly | 208 | . | . | . | . | T | T | . | −1.80 | 1.00 | . | . | F | 0.35 | 0.21 |
| Ser | 209 | . | . | . | . | . | T | C | −2.37 | 0.90 | . | * | F | 0.15 | 0.17 |
| Leu | 210 | . | . | . | B | . | . | C | −2.56 | 0.86 | . | . | . | −0.40 | 0.10 |
| Val | 211 | . | . | B | B | . | . | . | −2.60 | 1.16 | . | * | . | −0.60 | 0.07 |
| Ile | 212 | . | . | B | B | . | . | . | −2.97 | 1.37 | . | . | . | −0.60 | 0.04 |
| Val | 213 | . | . | B | B | . | . | . | −2.92 | 1.56 | . | . | . | −0.60 | 0.02 |
| Ile | 214 | . | . | B | B | . | . | . | −2.93 | 1.26 | . | . | . | −0.60 | 0.04 |
| Val | 215 | . | . | B | B | . | . | . | −2.98 | 1.10 | . | . | . | −0.60 | 0.09 |
| Cys | 216 | . | . | B | B | . | . | . | −2.47 | 1.06 | . | . | . | −0.60 | 0.09 |
| Ser | 217 | . | . | B | B | . | . | . | −2.39 | 0.84 | . | . | . | −0.60 | 0.13 |
| Thr | 218 | . | . | B | B | . | . | . | −2.42 | 0.84 | . | . | . | −0.60 | 0.14 |
| Val | 219 | . | . | B | B | . | . | . | −2.42 | 0.89 | . | . | . | −0.60 | 0.19 |
| Gly | 220 | . | . | B | B | . | . | . | −2.23 | 1.00 | . | . | . | −0.60 | 0.10 |
| Leu | 221 | . | . | B | B | . | . | . | −2.42 | 1.19 | . | * | . | −0.60 | 0.04 |
| Ile | 222 | . | . | B | B | . | . | . | −2.08 | 1.34 | * | . | . | −0.60 | 0.04 |
| Ile | 223 | . | . | B | B | . | . | . | −1.66 | 0.70 | * | * | . | −0.60 | 0.07 |
| Cys | 224 | . | . | B | B | . | . | . | −0.69 | 0.27 | . | . | . | −0.30 | 0.18 |
| Val | 225 | . | . | B | B | . | . | . | −0.30 | −0.41 | . | . | . | 0.30 | 0.49 |
| Lys | 226 | . | . | B | B | . | . | . | 0.30 | −1.10 | * | * | F | 1.24 | 1.40 |
| Arg | 227 | . | . | B | . | . | . | . | 1.30 | −1.36 | * | * | F | 1.78 | 4.04 |
| Arg | 228 | . | . | . | . | T | . | . | 1.84 | −1.93 | . | * | F | 2.52 | 10.65 |
| Lys | 229 | . | . | . | . | . | T | C | 2.51 | −2.14 | . | * | F | 2.86 | 5.27 |
| Pro | 230 | . | . | . | . | T | T | . | 2.51 | −2.14 | . | * | F | 3.40 | 4.49 |
| Arg | 231 | . | . | . | . | T | T | . | 1.61 | −1.50 | . | * | F | 3.06 | 1.70 |
| Gly | 232 | . | . | . | . | T | T | . | 1.54 | −0.86 | * | * | F | 2.57 | 0.63 |
| Asp | 233 | . | . | B | B | . | . | . | 0.58 | −0.86 | * | . | F | 1.43 | 0.82 |
| Val | 234 | . | . | B | B | . | . | . | −0.36 | −0.64 | * | * | F | 1.09 | 0.31 |
| Val | 235 | . | . | B | B | . | . | . | −1.00 | 0.04 | . | * | . | −0.30 | 0.22 |
| Lys | 236 | . | . | B | B | . | . | . | −1.41 | 0.26 | * | * | . | −0.30 | 0.10 |
| Val | 237 | . | . | B | B | . | . | . | −1.92 | 0.64 | * | * | . | −0.60 | 0.18 |
| Ile | 238 | . | . | B | B | . | . | . | −1.92 | 0.64 | * | . | . | −0.60 | 0.18 |
| Val | 239 | . | . | B | B | . | . | . | −0.96 | 0.40 | * | * | . | −0.30 | 0.15 |
| Ser | 240 | . | . | B | B | . | . | . | −0.06 | 0.40 | * | * | . | −0.08 | 0.40 |
| Val | 241 | . | . | B | B | . | . | . | 0.01 | −0.24 | * | * | . | 0.89 | 1.15 |
| Gln | 242 | . | . | B | B | . | . | . | 0.87 | −0.93 | * | . | F | 1.56 | 3.03 |
| Arg | 243 | . | . | . | B | . | . | C | 1.76 | −1.17 | . | . | F | 1.98 | 3.91 |
| Lys | 244 | . | . | . | B | . | . | C | 2.02 | −1.56 | . | * | F | 2.20 | 9.13 |
| Arg | 245 | . | A | . | . | . | . | C | 2.32 | −1.70 | . | * | F | 1.98 | 5.33 |
| Gln | 246 | . | A | . | . | . | . | C | 2.83 | −2.10 | . | * | F | 1.76 | 4.71 |
| Glu | 247 | . | A | . | . | . | . | C | 2.83 | −1.67 | . | * | F | 1.54 | 2.33 |
| Ala | 248 | . | A | . | . | . | . | C | 2.13 | −1.67 | . | * | F | 1.32 | 2.06 |
| Glu | 249 | . | A | . | . | . | . | C | 1.78 | −1.17 | . | * | F | 1.10 | 1.20 |
| Gly | 250 | . | A | . | . | . | . | C | 0.81 | −1.09 | * | * | F | 1.10 | 1.00 |
| Glu | 251 | A | A | . | B | . | . | . | −0.08 | −0.44 | . | * | F | 0.45 | 0.74 |
| Ala | 252 | A | A | . | B | . | . | . | −0.08 | −0.26 | . | * | F | 0.45 | 0.30 |
| Thr | 253 | A | A | . | B | . | . | . | −0.08 | −0.26 | * | * | . | 0.30 | 0.52 |
| Val | 254 | A | A | . | B | . | . | . | −0.89 | −0.19 | * | . | . | 0.30 | 0.30 |
| Ile | 255 | . | A | B | B | . | . | . | −0.54 | 0.50 | . | . | . | −0.60 | 0.25 |
| Glu | 256 | . | A | B | B | . | . | . | −1.13 | 0.40 | . | . | . | −0.30 | 0.30 |
| Ala | 257 | . | A | B | . | . | . | . | −0.76 | 0.41 | * | . | . | −0.60 | 0.41 |
| Leu | 258 | . | A | B | . | . | . | . | −0.66 | 0.20 | * | . | . | −0.30 | 0.89 |
| Gln | 259 | . | A | . | . | . | . | C | 0.20 | −0.06 | * | * | . | 0.78 | 0.80 |
| Ala | 260 | . | A | . | . | . | . | C | 0.23 | −0.06 | * | * | F | 1.36 | 1.32 |
| Pro | 261 | . | . | . | . | . | T | C | −0.08 | 0.09 | * | * | F | 1.44 | 1.19 |
| Pro | 262 | . | . | . | . | T | T | . | 0.20 | −0.11 | * | * | F | 2.37 | 0.99 |
| Asp | 263 | . | . | . | . | T | T | . | 0.16 | −0.03 | * | * | F | 2.80 | 1.42 |
| Val | 264 | . | . | B | . | . | T | . | −0.43 | 0.11 | * | * | F | 1.37 | 0.68 |
| Thr | 265 | . | . | B | B | . | . | . | −0.70 | 0.19 | * | . | F | 0.69 | 0.44 |
| Thr | 266 | . | . | B | B | . | . | . | −0.49 | 0.40 | . | . | . | 0.26 | 0.20 |
| Val | 267 | . | . | B | B | . | . | . | −0.28 | 0.40 | . | . | . | −0.02 | 0.46 |
| Ala | 268 | . | . | B | B | . | . | . | −0.59 | −0.24 | . | * | . | 0.30 | 0.55 |
| Val | 269 | . | . | B | B | . | . | . | −0.62 | −0.24 | . | . | . | 0.30 | 0.55 |
| Glu | 270 | . | . | B | B | . | . | . | −0.52 | −0.04 | . | . | F | 0.45 | 0.52 |
| Glu | 271 | . | . | B | B | . | . | . | −0.51 | −0.26 | * | . | F | 0.45 | 0.80 |
| Thr | 272 | . | . | B | B | . | . | . | −0.36 | −0.37 | * | . | F | 0.60 | 1.44 |
| Ile | 273 | . | . | B | B | . | . | . | −0.08 | −0.23 | * | . | F | 0.45 | 0.72 |
| Pro | 274 | . | . | B | B | . | . | . | 0.43 | 0.26 | * | * | F | −0.15 | 0.60 |
| Ser | 275 | . | . | . | B | T | . | . | 0.54 | 0.69 | * | * | F | −0.05 | 0.41 |
| Phe | 276 | . | . | . | B | T | . | . | 0.24 | 0.20 | * | * | F | 0.40 | 1.15 |
| Thr | 277 | . | . | . | B | T | . | . | 0.34 | −0.10 | * | . | F | 0.85 | 1.00 |
| Gly | 278 | . | . | . | . | T | . | . | 1.23 | −0.10 | * | * | F | 1.45 | 1.15 |
| Arg | 279 | . | . | . | . | . | . | C | 1.41 | −0.09 | * | * | F | 1.50 | 2.14 |
| Ser | 280 | . | . | . | . | . | T | C | 1.32 | −0.37 | * | * | F | 1.95 | 2.01 |
| Pro | 281 | . | . | . | . | . | T | C | 1.63 | −0.43 | * | * | . | 2.05 | 2.60 |
| Asn | 282 | . | . | . | . | T | T | . | 1.56 | −0.43 | * | . | . | 2.50 | 1.70 |
| His | 283 | . | . | . | . | . | T | C | 1.51 | −0.00 | * | . | . | 2.05 | 1.62 |

In another aspect, the invention provides an antibody that binds a peptide or polypeptide comprising an epitope-bearing portion of a polypeptide described herein. The epitope of this polypeptide portion is an immunogenic or antigenic epitope of a polypeptide of the invention. An “immunogenic epitope” is defined as a part of a protein that elicits an antibody response when the whole protein is the immunogen. On the other hand, a region of a protein molecule to which an antibody can bind is defined as an “antigenic epitope.” The number of immunogenic epitopes of a protein generally is less than the number of antigenic epitopes. See, for instance, Geysen et al., Proc. Natl. Acad. Sci. USA 81:3998-4002 (1983).

As to the selection of peptides or polypeptides bearing an antigenic epitope (i.e., that contain a region of a protein molecule to which an antibody can bind), it is well known in that art that relatively short synthetic peptides that mimic part of a protein sequence are routinely capable of eliciting an antiserum that reacts with the partially mimicked protein. See, for instance, Sutcliffe, J. G., Shinnick, T. M., Green, N. and Learner, R. A. (1983) Antibodies that react with predetermined sites on proteins. Science 219:660-666. Peptides capable of eliciting protein-reactive sera are frequently represented in the primary sequence of a protein, can be characterized by a set of simple chemical rules, and are confined neither to immunodominant regions of intact proteins nor to the amino or carboxyl terminals.

Antigenic epitope-bearing peptides and polypeptides are therefore useful to raise antibodies, including monoclonal antibodies, that bind to a TR2 polypeptide of the invention. See, for instance, Wilson et al., Cell 37:767-778 (1984) at 777. Antigenic epitope-bearing peptides and polypeptides preferably contain a sequence of at least seven, more preferably at least nine and most preferably between at least about 15 to about 30 amino acids contained within the amino acid sequence of SEQ ID NO:2.

Antibodies of the invention may bind one or more antigenic TR2 polypeptides or peptides including, but not limited to: a polypeptide comprising amino acid residues from about 14 to about 19 of SEQ ID NO:2; a polypeptide comprising amino acid residues from about 41 to about 45 of SEQ ID NO:2; a polypeptide comprising amino acid residues from about 108 to about 114 of SEQ ID NO:2; a polypeptide comprising amino acid residues from about 130 to about 134 of SEQ ID NO:2; a polypeptide comprising amino acid residues from about 150 to about 161 of SEQ ID NO:2; a polypeptide comprising amino acid residues from about 227 to about 232 of SEQ ID NO:2; a polypeptide comprising amino acid residues from about 242 to about 247 of SEQ ID NO:2; and/or a polypeptide comprising amino acid residues from about 279 to about 283 of SEQ ID NO:2. In this context “about” includes the particularly recited range, larger or smaller by several (5, 4, 3, 2, or 1) amino acids, at either terminus or at both termini. As indicated above, the inventors have determined that the above polypeptide fragments are antigenic regions of the TR2 protein. Epitope-bearing TR2 peptides and polypeptides may be produced by any conventional means. Houghten, R. A., “General method for the rapid solid-phase synthesis of large numbers of peptides: specificity of antigen-antibody interaction at the level of individual amino acids,” Proc. Natl. Acad. Sci. USA 82:5131-5135 (1985). This “Simultaneous Multiple Peptide Synthesis (SMPS)” process is further described in U.S. Pat. No. 4,631,211 to Houghten et al. (1986).

As one of skill in the art will appreciate, TR2 polypeptides and the epitope-bearing fragments thereof described herein (e.g., corresponding to all or a portion of the extracellular domain such as, for example, amino acid residues 37 to 200 of SEQ ID NO:2 can be combined with parts of the constant domain of immunoglobulins (IgG), resulting in chimeric polypeptides. These fusion proteins facilitate purification and show an increased half-life in vivo. This has been shown, e.g., for chimeric proteins consisting of the first two domains of the human CD4-polypeptide and various domains of the constant regions of the heavy or light chains of mammalian immunoglobulins (EPA 394,827; Traunecker et al., Nature 331:84-86 (1988)). Fusion proteins that have a disulfide-linked dimeric structure due to the IgG part can also be more efficient in binding and neutralizing other molecules than the monomeric TR2 protein or protein fragment alone (Fountoulakis et al., J Biochem 270:3958-3964 (1995)). Thus, antibodies of the invention may bind the TR2 moiety of fusion proteins that comprise all or a portion of a TR2 polypeptide.

Recombinant DNA technology known to those skilled in the art can be used to create novel mutant proteins or “muteins” including single or multiple amino acid substitutions, deletions, additions or fusion proteins. Such modified polypeptides can show, e.g., enhanced activity or increased stability. In addition, they may be purified in higher yields and show better solubility than the corresponding natural polypeptide, at least under certain purification and storage conditions. Antibodies of the present invention may also bind such modified TR2 polypeptides or TR2 polypeptide fragments or variants.

For instance, for many proteins, including the extracellular domain of a membrane associated protein or the mature form(s) of a secreted protein, it is known in the art that one or more amino acids may be deleted from the N-terminus or C-terminus without substantial loss of biological function, or loss of the ability to be bound by a specific antibody. For instance, Ron et al., J. Biol. Chem., 268:2984-2988 (1993) reported modified KGF proteins that had heparin binding activity even if 3, 8, or 27 amino-terminal amino acid residues were missing

However, even if deletion of one or more amino acids from the N-terminus of a protein results in modification or loss of one or more biological functions of the protein, other functional activities (e.g., biological activities, ability to multimerize, ability to bind TR2 ligand (e.g., LIGHT or LTα)) may still be retained. For example, the ability of shortened TR2 polypeptides to induce and/or bind to antibodies which recognize the complete or mature forms of the TR2 polypeptides generally will be retained when less than the majority of the residues of the complete or mature polypeptide are removed from the N-terminus. Whether a particular polypeptide lacking N-terminal residues of a complete polypeptide retains such immunologic activities can readily be determined by routine methods described herein and otherwise known in the art. It is not unlikely that a TR2 polypeptide with a large number of deleted N-terminal amino acid residues may retain some biological or immunogenic activities. In fact, peptides composed of as few as six TR2 amino acid residues may often evoke an immune response.

Accordingly, the present invention further provides antibodies that bind polypeptides having one or more residues deleted from the amino terminus of the TR2 amino acid sequence of SEQ ID NO:2 up to the glycine residue at position number 278 and polynucleotides encoding such polypeptides. In particular, the present invention provides antibodies that bind polypeptides comprising the amino acid sequence of residues n¹-283 of SEQ ID NO:2, where n¹ is an integer from 2 to 278 corresponding to the position of the amino acid residue in SEQ ID NO:2.

More in particular, the invention provides antibodies that bind polypeptides comprising, or alternatively consisting of, the amino acid sequence of residues of E-2 to H-283; P-3 to H-283; P-4 to H-283; G-5 to H-283; D-6 to H-283; W-7 to H-283; G-8 to H-283; P-9 to H-283; P-10 to H-283; P-11 to H-283; W-12 to H-283; R-13 to H-283; S-14 to H-283; T-15 to H-283; P-16 to H-283; K-17 to H-283; T-18 to H-283; D-19 to H-283; V-20 to H-283; L-21 to H-283; R-22 to H-283; L-23 to H-283; V-24 to H-283; L-25 to H-283; Y-26 to H-283; L-27 to H-283; T-28 to H-283; F-29 to H-283; L-30 to H-283; G-31 to H-283; A-32 to H-283; P-33 to H-283; C-34 to H-283; Y-35 to H-283; A-36 to H-283; P-37 to H-283; A-38 to H-283; L-39 to H-283; P-40 to H-283; S-41 to H-283; C-42 to H-283; K-43 to H-283; E-44 to H-283; D-45 to H-283; E-46 to H-283; Y-47 to H-283; P-48 to H-283; V-49 to H-283; G-50 to H-283; S-51 to H-283; E-52 to H-283 C-53 to H-283; C-54 to H-283; P-55 to H-283; K-56 to H-283; C-57 to H-283; S-58 to H-283; P-59 to H-283; G-60 to H-283; Y-61 to H-283; R-62 to H-283; V-63 to H-283; K-64 to H-283; E-65 to H-283; A-66 to H-283; C-67 to H-283; G-68 to H-283; E-69 to H-283; L-70 to H-283; T-71 to H-283; G-72 to H-283; T-73 to H-283; V-74 to H-283; C-75 to H-283; E-76 to H-283; P-77 to H-283; C-78 to H-283; P-79 to H-283; P-80 to H-283; G-81 to H-283; T-82 to H-283; Y-83 to H-283; 1-84 to H-283; A-85 to H-283; H-86 to H-283; L-87 to H-283; N-88 to H-283; G-89 to H-283; L-90 to H-283; S-91 to H-283; K-92 to H-283; C-93 to H-283; L-94 to H-283; Q-95 to H-283; C-96 to H-283; Q-97 to H-283; M-98 to H-283; C-99 to H-283; D-100 to H-283; P-101 to H-283; A-102 to H-283; M-103 to H-283; G-104 to H-283; L-105 to H-283; R-106 to H-283; A-107 to H-283; S-108 to H-283; R-109 to H-283; N-110 to H-283; C-111 to H-283; S-112 to H-283; R-113 to H-283; T-114 to H-283; E-115 to H-283; N-116 to H-283; A-117 to H-283; V-118 to H-283; C-119 to H-283; G-120 to H-283; C-121 to H-283; S-122 to H-283; P-123 to H-283; G-124 to H-283; H-125 to H-283; F-126 to H-283; C-127 to H-283; I-128 to H-283; V-129 to H-283; Q-130 to H-283; D-131 to H-283; G-132 to H-283; D-133 to H-283; H-134 to H-283; C-135 to H-283; A-136 to H-283; A-137 to H-283; C-138 to H-283; R-139 to H-283; A-140 to H-283; Y-141 to H-283; A-142 to H-283; T-143 to H-283; S-144 to H-283; S-145 to H-283; P-146 to H-283; G-147 to H-283; Q-148 to H-283; R-149 to H-283; V-150 to H-283; Q-151 to H-283; K-152 to H-283; G-153 to H-283; G-154 to H-283; T-155 to H-283; E-156 to H-283; S-157 to H-283; Q-158 to H-283; D-159 to H-283; T-160 to H-283; L-161 to H-283; C-162 to H-283; Q-163 to H-283; N-164 to H-283; C-165 to H-283; P-166 to H-283; P-167 to H-283; G-168 to H-283; T-169 to H-283; F-170 to H-283; S-171 to H-283; P-172 to H-283; N-173 to H-283; G-174 to H-283; T-175 to H-283; L-176 to H-283; E-177 to H-283; E-178 to H-283; C-179 to H-283; Q-180 to H-283; H-181 to H-283; Q-182 to H-283; T-183 to H-283; K-184 to H-283; C-185 to H-283; S-186 to H-283; W-187 to H-283; L-188 to H-283; V-189 to H-283; T-190 to H-283; K-191 to H-283; A-192 to H-283; G-193 to H-283; A-194 to H-283; G-195 to H-283; T-196 to H-283; S-197 to H-283; S-198 to H-283; S-199 to H-283; H-200 to H-283; W-201 to H-283; V-202 to H-283; W-203 to H-283; W-204 to H-283; F-205 to H-283; L-206 to H-283; S-207 to H-283; G-208 to H-283; S-209 to H-283; L-210 to H-283; V-211 to H-283; I-212 to H-283; V-213 to H-283; I-214 to H-283; V-215 to H-283; C-216 to H-283; S-217 to H-283; T-218 to H-283; V-219 to H-283; G-220 to H-283; L-221 to H-283; I-222 to H-283; I-223 to H-283; C-224 to H-283; V-225 to H-283; K-226 to H-283; R-227 to H-283; R-228 to H-283; K-229 to H-283; P-230 to H-283; R-231 to H-283; G-232 to H-283; D-233 to H-283; V-234 to H-283; V-235 to H-283; K-236 to H-283; V-237 to H-283; I-238 to H-283; V-239 to H-283; S-240 to H-283; V-241 to H-283; Q-242 to H-283; R-243 to H-283; K-244 to H-283; R-245 to H-283; Q-246 to H-283; E-247 to H-283; A-248 to H-283; E-249 to H-283; G-250 to H-283; E-251 to H-283; A-252 to H-283; T-253 to H-283; V-254 to H-283; I-255 to H-283; E-256 to H-283; A-257 to H-283; L-258 to H-283; Q-259 to H-283; A-260 to H-283; P-261 to H-283; P-262 to H-283; D-263 to H-283; V-264 to H-283; T-265 to H-283; T-266 to H-283; V-267 to H-283; A-268 to H-283; V-269 to H-283; E-270 to H-283; E-271 to H-283; T-272 to H-283; I-273 to H-283; P-274 to H-283; S-275 to H-283; F-276 to H-283; T-277 to H-283; and G-278 to H-283 of the TR2 sequence of SEQ ID NO:2.

In another embodiment, N-terminal deletions of the TR2 polypeptide can be described by the general formula n² to 200 where n² is a number from 2 to 195 corresponding to the amino acid sequence identified of SEQ ID NO:2. In specific embodiments, antibodies of the invention bind N terminal deletions of the TR2 comprising, or alternatively consisting of, the amino acid sequence of residues: E-2 to H-200; P-3 to H-200; P-4 to H-200; G-5 to H-200; D-6 to H-200; W-7 to H-200; G-8 to H-200; P-9 to H-200; P-10 to H-200; P-11 to H-200; W-12 to H-200; R-13 to H-200; S-14 to H-200; T-15 to H-200; P-16 to H-200; K-17 to H-200; T-18 to H-200; D-19 to H-200; V-20 to H-200; L-21 to H-200; R-22 to H-200; L-23 to H-200; V-24 to H-200; L-25 to H-200; Y-26 to H-200; L-27 to H-200; T-28 to H-200; F-29 to H-200; L-30 to H-200; G-31 to H-200; A-32 to H-200; P-33 to H-200; C-34 to H-200; Y-35 to H-200; A-36 to H-200; P-37 to H-200; A-38 to H-200; L-39 to H-200; P-40 to H-200; S-41 to H-200; C-42 to H-200; K-43 to H-200; E-44 to H-200; D-45 to H-200; E-46 to H-200; Y-47 to H-200; P-48 to H-200; V-49 to H-200; G-50 to H-200; S-51 to H-200; E-52 to H-200; C-53 to H-200; C-54 to H-200; P-55 to H-200; K-56 to H-200; C-57 to H-200; S-58 to H-200; P-59 to H-200; G-60 to H-200; Y-61 to H-200; R-62 to H-200; V-63 to H-200; K-64 to H-200; E-65 to H-200; A-66 to H-200; C-67 to H-200; G-68 to H-200; E-69 to H-200; L-70 to H-200; T-71 to H-200; G-72 to H-200; T-73 to H-200; V-74 to H-200; C-75 to H-200; E-76 to H-200; P-77 to H-200; C-78 to H-200; P-79 to H-200; P-80 to H-200; G-81 to H-200; T-82 to H-200; Y-83 to H-200; 1-84 to H-200; A-85 to H-200; H-86 to H-200; L-87 to H-200; N-88 to H-200; G-89 to H-200; L-90 to H-200; S-91 to H-200; K-92 to H-200; C-93 to H-200; L-94 to H-200; Q-95 to H-200; C-96 to H-200; Q-97 to H-200; M-98 to H-200; C-99 to H-200; D-100 to H-200; P-101 to H-200; A-102 to H-200; M-103 to H-200; G-104 to H-200; L-105 to H-200; R-106 to H-200; A-107 to H-200; S-108 to H-200; R-109 to H-200; N-110 to H-200; C-111 to H-200; S-112 to H-200; R-113 to H-200; T-114 to H-200; E-115 to H-200; N-1 16 to H-200; A-1 17 to H-200; V-1 18 to H-200; C-1 19 to H-200; G-120 to H-200; C-121 to H-200; S-122 to H-200; P-123 to H-200; G-124 to H-200; H-125 to H-200; F-126 to H-200; C-127 to H-200; I-128 to H-200; V-129 to H-200; Q-130 to H-200; D-131 to H-200; G-132 to H-200; D-133 to H-200; H-134 to H-200; C-135 to H-200; A-136 to H-200; A-137 to H-200; C-138 to H-200; R-139 to H-200; A-140 to H-200; Y-141 to H-200; A-142 to H-200; T-143 to H-200; S-144 to H-200; S-145 to H-200; P-146 to H-200; G-147 to H-200; Q-148 to H-200; R-149 to H-200; V-150 to H-200; Q-151 to H-200; K-152 to H-200; G-153 to H-200; G-154 to H-200; T-155 to H-200; E-156 to H-200; S-157 to H-200; Q-158 to H-200; D-159 to H-200; T-160 to H-200; L-161 to H-200; C-162 to H-200; Q-163 to H-200; N-164 to H-200; C-165 to H-200; P-166 to H-200; P-167 to H-200; G-168 to H-200; T-169 to H-200; F-170 to H-200; S-171 to H-200; P-172 to H-200; N-173 to H-200; G-174 to H-200; T-175 to H-200; L-176 to H-200; E-177 to H-200; E-178 to H-200; C-179 to H-200; Q-180 to H-200; H-181 to H-200; Q-182 to H-200; T-183 to H-200; K-184 to H-200; C-185 to H-200; S-186 to H-200; W-187 to H-200; L-188 to H-200; V-189 to H-200; T-190 to H-200; K-191 to H-200; A-192 to H-200; G-193 to H-200; A-194 to H-200; G-195 to H-200; of the TR2 extracellular domain sequence of SEQ ID NO:2.

As mentioned above, even if deletion of one or more amino acids from the C-terminus of a protein results in modification of loss of one or more biological functions of the protein, other functional activities (e.g., biological activities, ability to multimerize, ability to bind TR2 ligand (e.g., LIGHT and/or LTα)) may still be retained. For example the ability of the shortened TR2 polypeptide to induce and/or bind to antibodies which recognize the complete or mature forms of the TR2 polypeptide generally will be retained when less than the majority of the residues of the complete or mature polypeptide are removed from the C-terminus. Whether a particular polypeptide lacking C-terminal residues of a complete polypeptide retains such immunologic activities can readily be determined by routine methods described herein and otherwise known in the art. It is not unlikely that a TR2 polypeptide with a large number of deleted C-terminal amino acid residues may retain some biological or immunogenic activities. In fact, peptides composed of as few as six TR2 amino acid residues may often evoke an immune response.

In another embodiment, antibodies of the invention bind C-terminal deletions of the TR2 polypeptide that can be described by the general formula 37-m¹ where m¹ is a number from 43 to 282 corresponding to the amino acid sequence identified of SEQ ID NO:2. In specific embodiments, the invention provides antibodies that bind TR2 polypeptides comprising, or alternatively consisting of, the amino acid sequence of residues: P-37 to N-282; P-37 to P-281; P-37 to S-280; P-37 to R-279; P-37 to G-278; P-37 to T-277; P-37 to F-276; P-37 to S-275; P-37 to P-274; P-37 to 1-273; P-37 to T-272; P-37 to E-271; P-37 to E-270; P-37 to V-269; P-37 to A-268; P-37 to V-267; P-37 to T-266; P-37 to T-265; P-37 to V-264; P-37 to D-263; P-37 to P-262; P-37 to P-261; P-37 to A-260; P-37 to Q-259; P-37 to L-258; P-37 to A-257; P-37 to E-256; P-37 to I-255; P-37 to V-254; P-37 to T-253; P-37 to A-252; P-37 to E-251; P-37 to G-250; P-37 to E-249; P-37 to A-248; P-37 to E-247; P-37 to Q-246; P-37 to R-245; P-37 to K-244; P-37 to R-243; P-37 to Q-242; P-37 to V-241; P-37 to S-240; P-37 to V-239; P-37 to I-238; P-37 to V-237; P-37 to K-236; P-37 to V-235; P-37 to V-234; P-37 to D-233; P-37 to G-232; P-37 to R-231; P-37 to P-230; P-37 to K-229; P-37 to R-228; P-37 to R-227; P-37 to K-226; P-37 to V-225; P-37 to C-224; P-37 to 1-223; P-37 to 1-222; P-37 to L-221; P-37 to G-220; P-37 to V-219; P-37 to T-218; P-37 to S-217; P-37 to C-216; P-37 to V-215; P-37 to I-214; P-37 to V-213; P-37 to 1-212; P-37 to V-211; P-37 to L-210; P-37 to S-209; P-37 to G-208; P-37 to S-207; P-37 to L-206; P-37 to F-205; P-37 to W-204; P-37 to W-203; P-37 to V-202; P-37 to W-201; P-37 to H-200; P-37 to S-199; P-37 to S-198; P-37 to S-197; P-37 to T-196; P-37 to G-195; P-37 to A-194; P-37 to G-193; P-37 to A-192; P-37 to K-191; P-37 to T-190; P-37 to V-189; P-37 to L-188; P-37 to W-187; P-37, to S-186; P-37 to C-185; P-37 to K-184; P-37 to T-183; P-37 to Q-182; P-37 to H-181; P-37 to Q-180; P-37 to C-179; P-37 to E-178; P-37 to E-177; P-37 to L-176; P-37 to T-175; P-37 to G-174; P-37 to N-173; P-37 to P-172; P-37 to S-171; P-37 to F-170; P-37 to T-169; P-37 to G-168; P-37 to P-167; P-37 to P-166; P-37 to C-165; P-37 to N-164; P-37 to Q-163; P-37 to C-162; P-37 to L-161; P-37 to T-160; P-37 to D-159; P-37 to Q-158; P-37 to S-157; P-37 to E-156; P-37 to T-155; P-37 to G-154; P-37 to G-153; P-37 to K-152; P-37 to Q-151; P-37 to V-150; P-37 to R-149; P-37 to Q-148; P-37 to G-147; P-37 to P-146; P-37 to S-145; P-37 to S-144; P-37 to T-143; P-37 to A-142; P-37 to Y-141; P-37 to A-140; P-37 to R-139; P-37 to C-138; P-37 to A-137; P-37 to A-136; P-37 to C-135; P-37 to H-134; P-37 to D-133; P-37 to G-132; P-37 to D-131; P-37 to Q-130; P-37 to V-129; P-37 to I-128; P-37 to C-127; P-37 to F-126; P-37 to H-125; P-37 to G-124; P-37 to P-123; P-37 to S-122; P-37 to C-121; P-37 to0G-120; P-37 to C-119; P-37 to V-118; P-37 to A-117; P-37 to N-116; P-37 to E-115; P-37 to T-114; P-37 to R-113; P-37 to S-112; P-37 to C-111; P-37 to N-110; P-37 to R-109; P-37 to S-108; P-37 to A-107; P-37 to R-106; P-37 to L-105; P-37 to G-104; P-37 to M-103; P-37 to A-102; P-37 to P-101; P-37 to D-100; P-37 to C-99; P-37 to M-98; P-37 to Q-97; P-37 to C-96; P-37 to Q-95; P-37 to L-94; P-37 to C-93; P-37 to K-92; P-37 to S-91; P-37 to L-90; P-37 to G-89; P-37 to N-88; P-37 to L-87; P-37 to H-86; P-37 to A-85; P-37 to 1-84; P-37 to Y-83; P-37 to T-82; P-37 to G-81; P-37 to P-80; P-37 to P-79; P-37 to C-78; P-37 to P-77; P-37 to E-76; P-37 to C-75; P-37 to V-74; P-37 to T-73; P-37 to G-72; P-37 to T-71; P-37 to L-70; P-37 to E-69; P-37 to G-68; P-37 to C-67; P-37 to A-66; P-37 to E-65; P-37 to K-64; P-37 to V-63; P-37 to R-62; P-37 to Y-61; P-37 to G-60; P-37 to P-59; P-37 to S-58; P-37 to C-57; P-37 to K-56; P-37 to P-55; P-37 to C-54; P-37 to C-53; P-37 to E-52; P-37 to S-51; P-37 to G-50; P-37 to V-49; P-37 to P-48; P-37 to Y-47; P-37 to E-46; P-37 to D-45; P-37 to E-44; P-37 to K-43 of the TR2 sequence of SEQ ID NO:2.

The invention also provides antibodies that bind polypeptides having one or more amino acids deleted from both the amino and the carboxyl termini of a TR2 polypeptide, which may be described generally as having residues n¹-m¹ and/or n²-m¹ of SEQ ID NO:2, where n¹, n², and m¹ are integers as described above.

Preferably, antibodies of the present invention bind fragments of TR2 comprising a portion of the extracellular domain; i.e., within residues 37-200 of SEQ ID NO:2, since any portion therein is expected to be soluble.

It will be recognized in the art that some amino acid sequence of TR2 can be varied without significant effect of the structure or function of the protein. If such differences in sequence are contemplated, it should be remembered that there will be critical areas on the protein which determine activity. Such areas will usually comprise residues which make up the ligand binding site or which form tertiary structures which affect these domains.

Thus, the invention further includes antibodies that bind variations of the TR2 protein which show substantial TR2 protein activity or which include regions of TR2 such as the protein fragments discussed below. Such mutants include deletions, insertions, inversions, repeats, and type substitution. Guidance concerning which amino acid changes are likely to be phenotypically silent can be found in Bowie, J. U. et al., Science 247:1306-1310 (1990).

Thus, antibodies of the present invention may bind a fragment, derivative, or analog of the polypeptide of SEQ ID NO:2, or that encoded by the cDNA in ATCC deposit 97059. Such fragments, variants or derivatives may be (i) one in which at least one or more of the amino acid residues are substituted with a conserved or non-conserved amino acid residue (preferably a conserved amino acid residue(s), and more preferably at least one but less than ten conserved amino acid residues) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide, such as an IgG Fc fusion region peptide or leader or secretory sequence or a sequence which is employed for purification of the mature polypeptide or a proprotein sequence. Such fragments, derivatives and analogs are deemed to be within the scope of those skilled in the art from the teachings herein.

Of particular interest are substitutions of charged amino acids with another charged amino acid and with neutral or negatively charged amino acids. The latter results in proteins with reduced positive charge to improve the characteristics of the TR2 protein. The prevention of aggregation is highly desirable. Aggregation of proteins not only results in a loss of activity but can also be problematic when preparing pharmaceutical formulations, because they can be immunogenic. (Pinckard et al., Clin Exp. Immunol. 2:331-340 (1967); Robbins et al., Diabetes 36:838-845 (1987); Cleland et al. Crit. Rev. Therapeutic Drug Carrier Systems 10:307-377 (1993)).

The replacement of amino acids can also change the selectivity of binding to cell surface receptors. Ostade et al., Nature 361:266-268 (1993) describes certain mutations resulting in selective binding of TNF-alpha to only one of the two known types of TNF receptors. Thus, the antibodies of the present invention may bind a TR2 that contains one or more amino acid substitutions, deletions or additions, either from natural mutations or human manipulation.

As indicated, changes are preferably of a minor nature, such as conservative amino acid substitutions that do not significantly affect the folding or activity of the protein (see Table 3).

TABLE 3
Conservative Amino Acid Substitutions.

| | |
|---|---|
| Aromatic | Phenylalanine |
| | Tryptophan |
| | Tyrosine |
| Hydrophobic | Leucine |
| | Isoleucine |
| | Valine |
| Polar | Glutamine |
| | Asparagine |
| Basic | Arginine |
| | Lysine |
| | Histidine |
| Acidic | Aspartic Acid |
| | Glutamic Acid |
| Small | Alanine |
| | Serine |
| | Threonine |
| | Methionine |
| | Glycine |

In specific embodiments, the number of substitutions, additions or deletions in the amino acid sequence of SEQ ID NO:2 and/or any of the polypeptide fragments described herein (e.g., the extracellular domain or intracellular domain) is 75, 70, 60, 50, 40, 35, 30, 25, 20, 15, 10, 9, 8, 7, 6, 5, 4, 3, 2, 1 or 30-20, 20-15, 20-10, 15-10, 10-1, 5-10, 1-5, 1-3 or 1-2.

In specific embodiments, the antibodies of the invention bind TR2 polypeptides or fragments or variants thereof (especially a fragment comprising or alternatively consisting of, the extracellular soluble domain of TR2), that contains any one or more of the following conservative mutations in TR2: M1 replaced with A, G, I, L, S, T, or V; E2 replaced with D; G5 replaced with A, I, L, S, T, M, or V; D6 replaced with E; W7 replaced with F, or Y; G8 replaced with A, I, L, S, T, M, or V; W12 replaced with F, or Y; R13 replaced with H, or K; S14 replaced with A, G, I, L, T, M, or V; T15 replaced with A, G, I, L, S, M, or V; K17 replaced with H, or R; T18 replaced with A, G, I, L, S, M, or V; D19 replaced with E; V20 replaced with A, G, I, L, S, T, or M; L21 replaced with A, G, I, S, T, M, or V; R22 replaced with H, or K; L23 replaced with A, G, I, S, T, M, or V; V24 replaced with A, G, I, L, S, T, or M; L25 replaced with A, G, I, S, T, M, or V; Y26 replaced with F, or W; L27 replaced with A, G, I, S, T, M, or V; T28 replaced with A, G, I, L, S, M, or V; F29 replaced with W, or Y; L30 replaced with A, G, I, S, T, M, or V; G31 replaced with A, I, L, S, T, M, or V; A32 replaced with G, I, L, S, T, M, or V; Y35 replaced with F, or W; A36 replaced with G, I, L, S, T, M, or V; A38 replaced with G, I, L, S, T, M, or V; L39 replaced with A, G, I, S, T, M, or V; S41 replaced with A, G, I, L, T, M, or V; K43 replaced with H, or R; E44 replaced with D; D45 replaced with E; E46 replaced with D; Y47 replaced with F, or W; V49 replaced with A, G, I, L, S, T, or M; G50 replaced with A, I, L, S, T, M, or V; S51 replaced with A, G, I, L, T, M, or V; E52 replaced with D; K56 replaced with H, or R; S58 replaced with A, G, I, L, T, M, or V; G60 replaced with A, I, L, S, T, M, or V; Y61 replaced with F, or W; R62 replaced with H, or K; V63 replaced with A, G, I, L, S, T, or M; K64 replaced with H, or R; E65 replaced with D; A66 replaced with G, I, L, S, T, M, or V; G68 replaced with A, I, L, S, T, M, or V; E69 replaced with D; L70 replaced with A, G, I, S, T, M, or V; T71 replaced with A, G, I, L, S, M, or V; G72 replaced with A, I, L, S, T, M, or V; T73 replaced with A, G, I, L, S, M, or V; V74 replaced with A, G, I, L, S, T, or M; E76 replaced with D; G81 replaced with A, I, L, S, T, M, or V; T82 replaced with A, G, I, L, S, M, or V; Y83 replaced with F, or W; 184 replaced with A, G, L, S, T, M, or V; A85 replaced with G, I, L, S, T, M, or V; H86 replaced with K, or R; L87 replaced with A, G, I, S, T, M, or V; N88 replaced with Q; G89 replaced with A, I, L, S, T, M, or V; L90 replaced with A, G, I, S, T, M, or V; S91 replaced with A, G, I, L, T, M, or V; K92 replaced with H, or R; L94 replaced with A, G, I, S, T, M, or V; Q95 replaced with N; Q97 replaced with N; M98 replaced with A, G, I, L, S, T, or V; D100 replaced with E; A102 replaced with G, I, L, S, T, M, or V; M103 replaced with A, G, I, L, S, T, or V; G104 replaced with A, I, L, S, T, M, or V; L105 replaced with A, G, I, S, T, M, or V; R106 replaced with H, or K; A107 replaced with G, I, L, S, T, M, or V; S108 replaced with A, G, I, L, T, M, or V; R109 replaced with H, or K; N110 replaced with Q; S112 replaced with A, G. I, L, T, M, or V; R113 replaced with H, or K; T114 replaced with A, G, I, L, S, M, or V; E115 replaced with D; N116 replaced with Q; A117 replaced with G, I, L, S, T, M, or V; V118 replaced with A, G, I, L, S, T, or M; G120 replaced with A, I, L, S, T, M, or V; S122 replaced with A, G, I, L, T, M, or V; G124 replaced with A, I, L, S, T, M, or V; H125 replaced with K, or R; F126 replaced with W, or Y; I128 replaced with A, G, L, S, T, M, or V; V129 replaced with A, G, I, L, S, T, or M; Q130 replaced with N; D131 replaced with E; G132 replaced with A, I, L, S, T, M, or V; D133 replaced with E; H134 replaced with K, or R; A136 replaced with G, I, L, S, T, M, or V; A137 replaced with G, I, L, S, T, M, or V; R139 replaced with H, or K; A140 replaced with G, I, L, S, T, M, or V; Y141 replaced with F, or W; A142 replaced with G, I, L, S, T, M, or V; T143 replaced with A, G, I, L, S, M, or V; S144 replaced with A, G, I, L, T, M, or V; S145 replaced with A, G, I, L, T, M, or V; G147 replaced with A, I, L, S, T, M, or V; Q148 replaced with N; R149 replaced with H, or K; V150 replaced with A, G, I, L, S, T, or M; Q151 replaced with N; K152 replaced with H, or R; G153 replaced with A, I, L, S, T, M, or V; G154 replaced with A, I, L, S, T, M, or V; T155 replaced with A, G, I, L, S, M, or V; E156 replaced with D; S157 replaced with A, G, I, L, T, M, or V; Q158 replaced with N; D159 replaced with E; T160 replaced with A, G, I, L, S, M, or V; L161 replaced with A, G, I, S, T, M, or V; Q163 replaced with N; N164 replaced with Q; G168 replaced with A, I, L, S, T, M, or V; T169 replaced with A, G, I, L, S, M, or V; F170 replaced with W, or Y; S171 replaced with A, G, I, L, T, M, or V; N173 replaced with Q; G174 replaced with A, I, L, S, T, M, or V; T175 replaced with A, G, I, L, S, M, or V; L176 replaced with A, G, I, S, T, M, or V; E177 replaced with D; E178 replaced with D; Q180 replaced with N; H181 replaced with K, or R; Q182 replaced with N; T183 replaced with A, G, I, L, S, M, or V; K184 replaced with H, or R; S186 replaced with A, G, I, L, T, M, or V; W187 replaced with F, or Y; L188 replaced with A, G, I, S, T, M, or V; V189 replaced with A, G, I, L, S, T, or M; T190 replaced with A, G, I, L, S, M, or V; K191 replaced with H, or R; A192 replaced with G, I, L, S, T, M, or V; G193 replaced with A, I, L, S, T, M, or V; A194 replaced with G, I, L, S, T, M, or V; G195 replaced with A, I, L, S, T, M, or V; T196 replaced with A, G, I, L, S, M, or V; S197 replaced with A, G, I, L, T, M, or V; S198 replaced with A, G, I, L, T, M, or V; S199 replaced with A, G, I, L, T, M, or V; H200 replaced with K, or R; W201 replaced with F, or Y; V202 replaced with A, G, I, L, S, T, or M; W203 replaced with F, or Y; W204 replaced with F, or Y; F205 replaced with W, or Y; L206 replaced with A, G, I, S, T, M, or V; S207 replaced with A, G, I, L, T, M, or V; G208 replaced with A, I, L, S, T, M, or V; S209 replaced with A, G, I, L, T, M, or V; L210 replaced with A, G, I, S, T, M, or V; V211 replaced with A, G, I, L, S, T, or M; I212 replaced with A, G, L, S, T, M, or V; V213 replaced with A, G, I, L, S, T, or M; I214 replaced with A, G, L, S, T, M, or V; V215 replaced with A, G, I, L, S, T, or M; S217 replaced with A, G, I, L, T, M, or V; T218 replaced with A, G, I, L, S, M, or V; V219 replaced with A, G, I, L, S, T, or M; G220 replaced with A, I, L, S, T, M, or V; L221 replaced with A, G, I, S, T, M, or V; I222 replaced with A, G, L, S, T, M, or V; I223 replaced with A, G, L, S, T, M, or V; V225 replaced with A, G, I, L, S, T, or M; K226 replaced with H, or R; R227 replaced with H, or K; R228 replaced with H, or K; K229 replaced with H, or R; R231 replaced with H, or K; G232 replaced with A, I, L, S, T, M, or V; D233 replaced with E; V234 replaced with A, G, I, L, S, T, or M; V235 replaced with A, G, I, L, S, T, or M; K236 replaced with H, or R; V237 replaced with A, G, I, L, S, T, or M; I238 replaced with A, G, L, S, T, M, or V; V239 replaced with A, G, I, L, S, T, or M; S240 replaced with A, G, I, L, T, M, or V; V241 replaced with A, G, I, L, S, T, or M; Q242 replaced with N; R243 replaced with H, or K; K244 replaced with H, or R; R245 replaced with H, or K; Q246 replaced with N; E247 replaced with D; A248 replaced with G, I, L, S, T, M, or V; E249 replaced with D; G250 replaced with A, I, L, S, T, M, or V; E251 replaced with D; A252 replaced with G, I, L, S, T, M, or V; T253 replaced with A, G, I, L, S, M, or V; V254 replaced with A, G, I, L, S, T, or M; I255 replaced with A, G, L, S, T, M, or V; E256 replaced with D; A257 replaced with G, I, L, S, T, M, or V; L258 replaced with A, G, I, S, T, M, or V; Q259 replaced with N; A260 replaced with G, I, L, S, T, M, or V; D263 replaced with E; V264 replaced with A, G, I, L, S, T, or M; T265 replaced with A, G, I, L, S, M, or V; T266 replaced with A, G, I, L, S, M, or V; V267 replaced with A, G, I, L, S, T, or M; A268 replaced with G, I, L, S, T, M, or V; V269 replaced with A, G, I, L, S, T, or M; E270 replaced with D; E271 replaced with D; T272 replaced with A, G, I, L, S, M, or V; I273 replaced with A, G, L, S, T, M, or V; S275 replaced with A, G, I, L, T, M, or V; F276 replaced with W, or Y; T277 replaced with A, G, I, L, S, M, or V; G278 replaced with A, I, L, S, T, M, or V; R279 replaced with H, or K; S280 replaced with A, G, I, L, T, M, or V; N282 replaced with Q; H283 replaced with K, or R of SEQ ID NO:2.

In specific embodiments, the antibodies of the invention bind TR2 polypeptides or fragments or variants thereof (especially a fragment comprising or alternatively consisting of, the extracellular soluble domain of TR2), that contains any one or more of the following non-conservative mutations in TR2: MI replaced with D, E, H, K, R, N, Q, F, W, Y, P, or C; E2 replaced with H, K, R, A, G, I, L, S, T, M, V, N, Q, F, W, Y, P, or C; P3 replaced with D, E, H, K, R, A, G, I, L, S, T, M, V, N, Q, F, W, Y, or C; P4 replaced with D, E, H, K, R, A, G, I, L, S, T, M, V, N, Q, F, W, Y, or C; G5 replaced with D, E, H, K, R, N, Q, F, W, Y, P, or C; D6 replaced with H, K, R, A, G, I, L, S, T, M, V, N, Q, F, W, Y, P, or C; W7 replaced with D, E, H, K, R, N, Q, A, G, I, L, S, T, M, V, P, or C; G8 replaced with D, E, H, K, R, N, Q, F, W, Y, P, or C; P9 replaced with D, E, H, K, R, A, G, I, L, S, T, M, V, N, Q, F, W, Y, or C; P10 replaced with D, E, H, K, R, A, G, I, L, S, T, M, V, N, Q, F, W, Y, or C; P11 replaced with D, E, H, K, R, A, G, I, L, S, T, M, V, N, Q, F, W, Y, or C; W12 replaced with D, E, H, K, R, N, Q, A, G, I, L, S, T, M, V, P, or C; R13 replaced with D, E, A, G, I, L, S, T, M, V, N, Q, F, W, Y, P, or C; S14 replaced with D, E, H, K, R, N, Q, F, W, Y, P, or C; T15 replaced with D, E, H, K, R, N, Q, F, W, Y, P, or C; P16 replaced with D, E, H, K, R, A, G, I, L, S, T, M, V, N, Q, F, W, Y, or C; K17 replaced with D, E, A, G, I, L, S, T, M, V, N, Q, F, W, Y, P, or C; T18 replaced with D, E, H, K, R, N, Q, F, W, Y, P, or C; D19 replaced with H, K, R, A, G, I, L, S, T, M, V, N, Q, F, W, Y, P, or C; V20 replaced with D, E, H, K, R, N, Q, F, W, Y, P, or C; L21 replaced with D, E, H, K, R, N, Q, F, W, Y, P, or C; R22 replaced with D, E, A, G, I, L, S, T, M, V, N, Q, F, W, Y, P, or C; L23 replaced with D, E, H, K, R, N, Q, F, W, Y, P, or C; V24 replaced with D, E, H, K, R, N, Q, F, W, Y, P, or C; L25 replaced with D, E, H, K, R, N, Q, F, W, Y, P, or C; Y26 replaced with D, E, H, K, R, N, Q, A, G, I, L, S, T, M, V, P, or C; L27 replaced with D, E, H, K, R, N, Q, F, W, Y, P, or C; T28 replaced with D, E, H, K, R, N, Q, F, W, Y, P, or C; F29 replaced with D, E, H, K, R, N, Q, A, G, I, L, S, T, M, V, P, or C; L30 replaced with D, E, H, K, R, N, Q, F, W, Y, P, or C; G31 replaced with D, E, H, K, R, N, Q, F, W, Y, P, or C; A32 replaced with D, E, H, K, R, N, Q, F, W, Y, P, or C; P33 replaced with D, E, H, K, R, A, G, I, L, S, T, M, V, N, Q, F, W, Y, or C; C34 replaced with D, E, H, K, R, A, G, I, L, S, T, M, V, N, Q, F, W, Y, or P; Y35 replaced with D, E, H, K, R, N, Q, A, G, I, L, S, T, M, V, P, or C; A36 replaced with D, E, H, K, R, N, Q, F, W, Y, P, or C; P37 replaced with D, E, H, K, R, A, G, I, L, S, T, M, V, N, Q, F, W, Y, or C; A38 replaced with D, E, H, K, R, N, Q, F, W, Y, P, or C; L39 replaced with D, E, H, K, R, N, Q, F, W, Y, P, or C; P40 replaced with D, E, H, K, R, A, G, I, L, S, T, M, V, N, Q, F, W, Y, or C; S41 replaced with D, E, H, K, R, N, Q, F, W, Y, P, or C; C42 replaced with D, E, H, K, R, A, G, I, L, S, T, M, V, N, Q, F, W, Y, or P; K43 replaced with D, E, A, G, I, L, S, T, M, V, N, Q, F, W, Y, P, or C; E44 replaced with H, K, R, A, G, I, L, S, T, M, V, N, Q, F, W, Y, P, or C; D45 replaced with H, K, R, A, G, I, L, S, T, M, V, N, Q, F, W, Y, P, or C; E46 replaced with H, K, R, A, G, I, L, S, T, M, V, N, Q, F, W, Y, P, or C; Y47 replaced with D, E, H, K, R, N, Q, A, G, I, L, S, T, M, V, P, or C; P48 replaced with D, E, H, K, R, A, G, I, L, S, T, M, V, N, Q, F, W, Y, or C; V49 replaced with D, E, H, K, R, N, Q, F, W, Y, P, or C; G50 replaced with D, E, H, K, R, N, Q, F, W, Y, P, or C; S51 replaced with D, E, H, K, R, N, Q, F, W, Y, P, or C; E52 replaced with H, K, R, A, G, I, L, S, T, M, V, N, Q, F, W, Y, P, or C; C53 replaced with D, E, H, K, R, A, G, I, L, S, T, M, V, N, Q, F, W, Y, or P; C54 replaced with D, E, H, K, R, A, G, I, L, S, T, M, V, N, Q, F, W, Y, or P; P55 replaced with D, E, H, K, R, A, G, I, L, S, T, M, V, N, Q, F, W, Y, or C; K56 replaced with D, E, A, G, I, L, S, T, M, V, N, Q, F, W, Y, P, or C; C57 replaced with D, E, H, K, R, A, G, I, L, S, T, M, V, N, Q, F, W, Y, or P; S58 replaced with D, E, H, K, R, N, Q, F, W, Y, P, or C; P59 replaced with D, E, H, K, R, A, G, I, L, S, T, M, V, N, Q, F, W, Y, or C; G60 replaced with D, E, H, K, R, N, Q, F, W, Y, P, or C; Y61 replaced with D, E, H, K, R, N, Q, A, G, I, L, S, T, M, V, P, or C; R62 replaced with D, E, A, G, I, L, S, T, M, V, N, Q, F, W, Y, P, or C; V63 replaced with D, E, H, K, R, N, Q, F, W, Y, P, or C; K64 replaced with D, E, A, G, I, L, S, T, M, V, N, Q, F, W, Y, P, or C; E65 replaced with H, K, R, A, G, I, L, S, T, M, V, N, Q, F, W, Y, P, or C; A66 replaced with D, E, H, K, R, N, Q, F, W, Y, P, or C; C67 replaced with D, E, H, K, R, A, G, I, L, S, T, M, V, N, Q, F, W, Y, or P; G68 replaced with D, E, H, K, R, N, Q, F, W, Y, P, or C; E69 replaced with H, K, R, A, G, I, L, S, T, M, V, N, Q, F, W, Y, P, or C; L70 replaced with D, E, H, K, R, N, Q, F, W, Y, P, or C; T71 replaced with D, E, H, K, R, N, Q, F, W, Y, P, or C; G72 replaced with D, E, H, K, R, N, Q, F, W, Y, P, or C; T73 replaced with D, E, H, K, R, N, Q, F, W, Y, P, or C; V74 replaced with D, E, H, K, R, N, Q, F, W, Y, P, or C; C75 replaced with D, E, H, K, R, A, G, I, L, S, T, M, V, N, Q, F, W, Y,or P; E76 replaced with H, K, R, A, G, I, L, S, T, M, V, N, Q, F, W, Y, P, or C; P77 replaced with D, E, H, K, R, A, G, I, L, S, T, M, V, N, Q, F, W, Y, or C; C78 replaced with D, E, H, K, R, A, G, I, L, S, T, M, V, N, Q, F, W, Y, or P; P79 replaced with D, E, H, K, R, A, G, I, L, S, T, M, V, N, Q, F, W, Y, or C; P80 replaced with D, E, H, K, R, A, G, I, L, S, T, M, V, N, Q, F, W, Y, or C; G81 replaced with D, E, H, K, R, N, Q, F, W, Y, P, or C; T82 replaced with D, E, H, K, R, N, Q, F, W, Y, P, or C; Y83 replaced with D, E, H, K, R, N, Q, A, G, I, L, S, T, M, V, P, or C; I84 replaced with D, E, H, K, R, N, Q, F, W, Y, P, or C; A85 replaced with D, E, H, K, R, N, Q, F, W, Y, P, or C; H86 replaced with D, E, A, G, I, L, S, T, M, V, N, Q, F, W, Y, P, or C; L87 replaced with D, E, H, K, R, N, Q, F, W, Y, P, or C; N88 replaced with D, E, H, K, R, A, G, I, L, S, T, M, V, F, W, Y, P, or C; G89 replaced with D, E, H, K, R, N, Q, F, W, Y, P, or C; L90 replaced with D, E, H, K, R, N, Q, F, W, Y, P, or C; S91 replaced with D, E, H, K, R, N, Q, F, W, Y, P, or C; K92 replaced with D, E, A, G, I, L, S, T, M, V, N, Q, F, W, Y, P, or C; C93 replaced with D, E, H, K, R, A, G, I, L, S, T, M, V, N, Q, F, W, Y, or P; L94 replaced with D, E, H, K, R, N, Q, F, W, Y, P, or C; Q95 replaced with D, E, H, K, R, A, G, I, L, S, T, M, V, F, W, Y, P, or C; C96 replaced with D, E, H, K, R, A, G, I, L, S, T, M, V, N, Q, F, W, Y, or P; Q97 replaced with D, E, H, K, R, A, G, I, L, S, T, M, V, F, W, Y, P, or C; M98 replaced with D, E, H, K, R, N, Q, F, W, Y, P, or C; C99 replaced with D, E, H, K, R, A, G, I, L, S, T, M, V, N, Q, F, W, Y, or P; D100 replaced with H, K, R, A, G, I, L, S, T, M, V, N, Q, F, W, Y, P, or C; P101 replaced with D, E, H, K, R, A, G, I, L, S, T, M, V, N, Q, F, W, Y, or C; A102 replaced with D, E, H, K, R, N, Q, F, W, Y, P, or C; M103 replaced with D, E, H, K, R, N, Q, F, W, Y, P, or C; G104 replaced with D, E, H, K, R, N, Q, F, W, Y, P, or C; L105 replaced with D, E, H, K, R, N, Q, F, W, Y, P, or C; R106 replaced with D, E, A, G, I, L, S, T, M, V, N, Q, F, W, Y, P, or C; A107 replaced with D, E, H, K, R, N, Q, F, W, Y, P, or C; S108 replaced with D, E, H, K, R, N, Q, F, W, Y, P, or C; R109 replaced with D, E, A, G, I, L, S, T, M, V, N, Q, F, W, Y, P, or C; N110 replaced with D, E, H, K, R, A, G, I, L, S, T, M, V, F, W, Y, P, or C; C111 replaced with D, E, H, K, R, A, G, I, L, S, T, M, V,N, Q, F, W, Y, or P; S112 replaced with D, E, H, K, R, N, Q, F, W, Y, P, or C; R113 replaced with D, E, A, G, I, L, S, T, M, V, N, Q, F, W, Y, P, or C; T114 replaced with D, E, H, K, R, N, Q, F, W, Y, P, or C; E115 replaced with H, K, R, A, G, I, L, S, T, M, V, N, Q, F, W, Y, P, or C; N116 replacedwith D, E, H, K, R, A, G, I, L, S, T, M, V, F, W, Y, P, or C; A117 replaced with D, E, H, K, R, N, Q, F, W, Y, P, or C; V118 replaced with D, E, H, K, R, N, Q, F, W, Y, P, or C; C119 replaced with D, E, H, K, R, A, G, I, L, S, T, M, V, N, Q, F, W, Y, or P; G120 replaced with D, E, H, K, R, N, Q, F, W, Y, P, or C; C121 replaced with D, E, H, K, R, A, G, I, L, S, T, M, V, N, Q, F, W, Y, or P; S122 replaced with D, E, H, K, R, N, Q, F, W, Y, P, or C; P123 replaced with D, E, H, K, R, A, G, I, L, S, T, M, V, N, Q, F, W, Y, or C; G124 replaced with D, E, H, K, R, N, Q, F, W, Y, P, or C; H125 replaced with D, E, A, G, I, L, S, T, M, V, N, Q, F, W, Y, P, or C; F126 replaced with D, E, H, K, R, N, Q, A, G, I, L, S, T, M, V, P, or C; C127 replaced with D, E, H, K, R, A, G, I, L, S, T, M, V, N, Q, F, W, Y, or P; 1128 replaced with D, E, H, K, R, N, Q, F, W, Y, P, or C; V129 replaced with D, E, H, K, R, N, Q, F, W, Y, P, or C; Q130 replaced with D, E, H, K, R, A, G, I, L, S, T, M, V, F, W, Y, P, or C; D131 replaced with H, K, R, A, G, I, L, S, T, M, V, N, Q, F, W, Y, P, or C; G132 replaced with D, E, H, K, R, N, Q, F, W, Y, P, or C; D133 replaced with H, K, R, A, G, I, L, S, T, M, V, N, Q, F, W, Y, P, or C; H134 replaced with D, E, A, G, I, L, S, T, M, V, N, Q, F, W, Y, P, or C; C135 replaced with D, E, H, K, R, A, G, I, L, S, T, M, V, N, Q, F, W, Y, or P; A136 replaced with D, E, H, K, R, N, Q, F, W, Y, P, or C; A137 replaced with D, E, H, K, R, N, Q, F, W, Y, P, or C; C138 replaced with D, E, H, K, R, A, G, I, L, S, T, M, V, N, Q, F, W, Y, or P; R139 replaced with D, E, A, G, I, L, S, T, M, V, N, Q, F, W, Y, P, or C; A140 replaced with D, E, H, K, R, N, Q, F, W, Y, P, or C; Y141 replaced with D, E, H, K, R, N, Q, A, G, I, L, S, T, M, V, P, or C; A142 replaced with D, E, H, K, R, N, Q, F, W, Y, P, or C; T143 replaced with D, E, H, K, R, N, Q, F, W, Y, P, or C; S144 replaced with D, E, H, K, R, N, Q, F, W, Y, P, or C; S145 replaced with D, E, H, K, R, N, Q, F, W, Y, P, or C; P146 replaced with D, E, H, K, R, A, G, I, L, S, T, M, V, N, Q, F, W, Y, or C; G147 replaced with D, E, H, K, R, N, Q, F, W, Y, P, or C; Q148 replaced with D, E, H, K, R, A, G, I, L, S, T, M, V, F, W, Y, P, or C; R149 replaced with D, E, A, G, I, L, S, T, M, V, N, Q, F, W, Y, P, or C; V150 replaced with D, E, H, K, R, N, Q, F, W, Y, P, or C; Q151 replaced with D, E, H, K, R, A, G, I, L, S, T, M, V, F, W, Y, P, or C; K152 replaced with D, E, A, G, I, L, S, T, M, V, N, Q, F, W, Y, P, or C; G153 replaced with D, E, H, K, R, N, Q, F, W, Y, P, or C; G154 replaced with D, E, H, K, R, N, Q, F, W, Y, P, or C; T155 replaced with D, E, H, K, R, N, Q, F, W, Y, P, or C; E156 replaced with H, K, R, A, G, I, L, S, T, M, V, N, Q, F, W, Y, P, or C; S157 replaced with D, E, H, K, R, N, Q, F, W, Y, P, or C; Q158 replaced with D,E, H, K, R, A, G, I, L, S, T, M, V, F, W, Y, P, or C; D159 replaced with H, K, R, A, G, I, L, S, T, M, V, N, Q, F, W, Y, P, or C; T160 replaced with D, E, H, K, R, N, Q, F, W, Y, P, or C; L161 replaced with D, E, H, K, R, N, Q, F, W, Y, P, or C; C162 replaced with D, E, H, K, R, A, G, I, L, S, T, M, V, N, Q, F, W, Y, or P; Q163 replaced with D, E, H, K, R, A, G, I, L, S, T, M, V, F, W, Y, P, or C; N164 replaced with D, E, H, K, R, A, G, I, L, S, T, M, V, F, W, Y, P, or C; C165 replaced with D, E, H, K, R, A, G, I, L, S, T, M, V, N, Q, F, W, Y, or P; P166 replaced with D, E, H, K, R, A, G, I, L, S, T, M, V, N, Q, F, W, Y, or C; P167 replaced with D, E, H, K, R, A, G, I, L, S, T, M, V, N, Q, F, W, Y, or C; G168 replaced with D, E, H, K, R, N, Q, F, W, Y, P, or C; T169 replaced with D, E, H, K, R, N, Q, F, W, Y, P, or C; F170 replaced with D, E, H, K, R, N, Q, A, G, I, L, S, T, M, V, P, or C; S171 replaced with D, E, H, K, R, N, Q, F, W, Y, P, or C; P172 replaced with D, E, H, K, R, A, G, I, L, S, T, M, V, N, Q, F, W, Y, or C; N173 replaced with D, E, H, K, R, A, G, I, L, S, T, M, V, F, W, Y, P, or C; G174 replaced with D, E, H, K, R, N, Q, F, W, Y, P, or C; T175 replaced with D, E, H, K, R, N, Q, F, W, Y, P, or C; L176 replaced with D, E, H, K, R, N, Q, F, W, Y, P, or C; E177 replaced with H, K, R, A, G, I, L, S, T, M, V, N, Q, F, W, Y, P, or C; E178 replaced with H, K, R, A, G,I, L, S, T, M, V, N, Q, F, W, Y, P, or C; C179 replaced with D, E, H, K, R, A, G, I, L, S, T, M, V, N, Q, F, W, Y, or P; Q180 replaced with D, E, H, K, R, A, G, I, L, S, T, M, V, F, W, Y, P, or C; H181 replaced with D, E, A, G, I, L, S, T, M, V, N, Q, F, W, Y, P, or C; Q182 replaced with D, E, H, K, R, A, G, I, L, S, T, M, V, F, W, Y, P, or C; T183 replaced with D, E, H, K, R, N, Q, F, W, Y, P, or C; K184 replaced with D, E, A, G, I, L, S, T, M, V, N, Q, F, W, Y, P, or C; C185 replaced with D, E, H, K, R, A, G, I, L, S, T, M, V, N, Q, F, W, Y, or P; S186 replaced with D, E, H, K, R, N, Q, F, W, Y, P, or C; W187 replaced with D, E, H, K, R, N, Q, A, G, I, L, S, T, M, V, P, or C; L188 replaced with D, E, H, K, R, N, Q, F, W, Y, P, or C; V189 replaced with D, E, H, K, R, N, Q, F, W, Y, P, or C; T190 replaced with D, E, H, K, R, N, Q, F, W, Y, P, or C; K191 replaced with D, E, A, G,I, L, S, T, M, V, N, Q, F, W, Y, P, or C; A192 replaced with D, E, H, K, R, N, Q, F, W, Y, P, or C; G193 replaced with D, E, H, K, R, N, Q, F, W, Y, P, or C; A194 replaced with D, E, H, K, R, N, Q, F, W, Y, P, or C; G195 replaced with D, E, H, K, R, N, Q, F, W, Y, P, or C; T196 replaced with D, E, H, K, R, N, Q, F, W, Y, P, or C; S197 replaced with D, E, H, K, R, N, Q, F, W, Y, P, or C; S 198 replaced with D, E, H, K, R, N, Q, F, W, Y, P, or C; S199 replaced with D, E, H, K, R, N, Q, F, W, Y, P, or C; H200 replaced with D, E, A, G, I, L, S, T, M, V, N, Q, F, W, Y, P, or C; W201 replaced with D, E, H, K, R, N, Q, A, G, I, L, S, T, M, V, P, or C; V202 replaced with D, E, H, K, R, N, Q, F, W, Y, P, or C; W203 replaced with D, E, H, K, R, N, Q, A, G, I, L, S, T, M, V, P, or C; W204 replaced with D, E, H, K, R, N, Q, A, G, I, L, S, T, M, V, P, or C; F205 replaced with D, E, H, K, R, N, Q, A, G, I, L, S, T, M, V, P, or C; L206 replaced with D, E, H, K, R, N, Q, F, W, Y, P, or C; S207 replaced with D, E, H, K, R, N, Q, F, W, Y, P, or C; G208 replaced with D, E, H, K, R, N, Q, F, W, Y, P, or C; S209 replaced with D, E, H, K, R, N, Q, F, W, Y, P, or C; L210 replaced with D, E, H, K, R, N, Q, F, W, Y, P, or C; V211 replaced with D, E, H, K, R, N, Q, F, W, Y, P, or C; I212 replaced with D, E, H, K, R, N, Q, F, W, Y, P, or C; V213 replaced with D, E, H, K, R, N, Q, F, W, Y, P, or C; I214 replaced with D, E, H, K, R, N, Q, F, W, Y, P, or C; V215 replaced with D, E, H, K, R, N, Q, F, W, Y, P, or C; C216 replaced with D, E, H, K, R, A, G, I, L, S, T, M, V, N, Q, F, W, Y, or P; S217 replaced with D, E, H, K, R, N, Q, F, W, Y, P, or C; T218 replaced with D, E, H, K, R, N, Q, F, W, Y, P, or C; V219 replaced with D, E, H, K, R, N, Q, F, W, Y, P, or C; G220 replaced with D, E, H, K, R, N, Q, F, W, Y, P, or C; L221 replaced with D, E, H, K, R, N, Q, F, W, Y, P, or C; I222 replaced with D, E, H, K, R, N, Q, F, W, Y, P, or C; I223 replaced with D, E, H, K, R, N, Q, F, W, Y, P, or C; C224 replaced with D, E, H, K, R, A, G, I, L, S, T, M, V, N, Q, F, W, Y, or P; V225 replaced with D, E, H, K, R, N, Q, F, W, Y, P, or C; K226 replaced with D, E, A, G, I, L, S, T, M, V, N, Q, F, W, Y, P, or C; R227 replaced with D, E, A, G, I, L, S, T, M, V, N, Q, F, W, Y, P, or C; R228 replaced with D, E, A, G, I, L, S, T, M, V, N, Q, F, W, Y, P, or C; K229 replaced with D, E, A, G, I, L, S, T, M, V, N, Q, F, W, Y, P, or C; P230 replaced with D, E, H, K, R, A, G, I, L, S, T, M, V, N, Q, F, W, Y, or C; R231 replaced with D, E, A, G, I, L, S, T, M, V, N, Q, F, W, Y, P, or C; G232 replaced with D, E, H, K, R, N, Q, F, W, Y, P, or C; D233 replaced with H, K, R, A, G, I, L, S, T, M, V, N, Q, F, W, Y, P, or C; V234 replaced with D, E, H, K, R, N, Q, F, W, Y, P, or C; V235 replaced with D, E, H, K, R, N, Q, F, W, Y, P, or C; K236 replaced with D, E, A, G, I, L, S, T, M, V, N, Q, F, W, Y, P, or C; V237 replaced with D, E, H, K, R, N, Q, F, W, Y, P, or C; I238 replaced with D, E, H, K, R, N, Q, F, W, Y, P, or C; V239 replaced with D, E, H, K, R, N, Q, F, W, Y, P, or C; S240 replaced with D, E, H, K, R, N, Q, F, W, Y, P, or C; V241 replaced with D, E, H, K, R, N, Q, F, W, Y, P, or C; Q242 replaced with D, E, H, K, R, A, G, I, L, S, T, M, V, F, W, Y, P, or C; R243 replaced with D, E, A, G, I, L, S, T, M, V, N, Q, F, W, Y, P, or C; K244 replaced with D, E, A, G, I, L, S, T, M, V, N, Q, F, W, Y, P, or C; R245 replaced with D, E, A, G, I, L, S, T, M, V, N, Q, F, W, Y, P, or C; Q246 replaced with D, E, H, K, R, A, G, I, L, S, T, M, V, F, W, Y, P, or C; E247 replaced with H, K, R, A, G, I, L, S, T, M, V, N, Q, F, W, Y, P, or C; A248 replaced with D, E, H, K, R, N, Q, F, W, Y, P, or C; E249 replaced with H, K, R, A, G, I, L, S, T, M, V, N, Q, F, W, Y, P, or C; G250 replaced with D, E, H, K, R, N, Q, F, W, Y, P, or C; E251 replaced with H, K, R, A, G, I, L, S, T, M, V, N, Q, F, W, Y, P, or C; A252 replaced with D, E, H, K, R, N, Q, F, W, Y, P, or C; T253 replaced with D, E, H, K, R, N, Q, F, W, Y, P, or C; V254 replaced with D, E, H, K, R, N, Q, F, W, Y, P, or C; I255 replaced with D, E, H, K, R, N, Q, F, W, Y, P, or C; E256 replaced with H, K, R, A, G, I, L, S, T, M, V, N, Q, F, W, Y, P, or C; A257 replaced with D, E, H, K, R, N, Q, F, W, Y, P, or C; L258 replaced with D, E, H, K, R, N, Q, F, W, Y, P, or C; Q259 replaced with D, E, H, K, R, A, G, I, L, S, T, M, V, F, W, Y, P, or C; A260 replaced with D, E, H, K, R, N, Q, F, W, Y, P, or C; P261 replaced with D, E, H, K, R, A, G, I, L, S, T, M, V, N, Q, F, W, Y, or C; P262 replaced with D, E, H, K, R, A, G, I, L, S, T, M, V, N, Q, F, W, Y, or C; D263 replaced with H, K, R, A, G, I, L, S, T, M, V, N, Q, F, W, Y, P, or C; V264 replaced with D, E, H, K, R, N, Q, F, W, Y, P, or C; T265 replaced with D, E, H, K, R, N, Q, F, W, Y, P, or C; T266 replaced with D, E, H, K, R, N, Q, F, W, Y, P, or C; V267 replaced with D, E, H, K, R, N, Q, F, W, Y, P, or C; A268 replaced with D, E, H, K, R, N, Q, F, W, Y, P, or C; V269 replaced with D, E, H, K, R, N, Q, F, W, Y, P, or C; E270 replaced with H, K, R, A, G, I, L, S, T, M, V, N, Q, F, W, Y, P, or C; E271 replaced with H, K, R, A, G, I, L, S, T, M, V, N, Q, F, W, Y, P, or C; T272 replaced with D, E, H, K, R, N, Q, F, W, Y, P, or C; I273 replaced with D, E, H, K, R, N, Q, F, W, Y, P, or C; P274 replaced with D, E, H, K, R, A, G, I, L, S, T, M, V, N, Q, F, W, Y, or C; S275 replaced with D, E, H, K, R, N, Q, F, W, Y, P, or C; F276 replaced with D, E, H, K, R, N, Q, A, G, I, L, S, T, M, V, P, or C; T277 replaced with D, E, H, K, R, N, Q, F, W, Y, P, or C; G278 replaced with D, E, H, K, R, N, Q, F, W, Y, P, or C; R279 replaced with D, E, A, G, I, L, S, T, M, V, N, Q, F, W, Y, P, or C; S280 replaced with D, E, H, K, R, N, Q, F, W, Y, P, or C; P281 replaced with D, E, H, K, R, A, G, I, L, S, T, M, V, N, Q, F, W, Y, or C; N282 replaced with D, E, H, K, R, A, G, I, L, S, T, M, V, F, W, Y, P, or C; H283 replaced with D, E, A, G, I,L, S, T, M, V, N, Q, F, W, Y, P, or C of SEQ ID NO:2.

Amino acids in the TR2 protein of the present invention that are essential for function can be identified by methods known in the art, such as site-directed mutagenesis or alanine-scanning mutagenesis (Cunningham and Wells, Science 244:1081-1085 (1989)). The latter procedure introduces single alanine mutations at every residue in the molecule. The resulting mutant molecules are then tested for biological activity such as receptor binding or in vitro, or in vivo proliferative activity. Sites that are critical for ligand-receptor binding can also be determined by structural analysis such as crystallization, nuclear magnetic resonance or photoaffinity labeling (Smith et al., J. Mol. Biol. 224:899-904 (1992) and de Vos et al. Science 255:306-312 (1992)). In preferred embodiments, antibodies of the present invention bind regions of TR2 that are essential for TR2 function. In other preferred embodiments, antibodies of the present invention bind regions of TR2 that are essential for TR2 function and inhibit or abolish TR2 function. In other preferred embodiments, antibodies of the present invention bind regions of TR2 that are essential for TR2 function and enhance TR2 function.

Additionally, protein engineering may be employed to improve or alter the characteristics of TR2 polypeptides. Recombinant DNA technology known to those skilled in the art can be used to create novel mutant proteins or muteins including single or multiple amino acid substitutions, deletions, additions or fusion proteins. Such modified polypeptides can show, e.g., enhanced activity or increased stability. In addition, they may be purified in higher yields and show better solubility than the corresponding natural polypeptide, at least under certain purification and storage conditions. Antibodies of the present invention may bind such modified TR2 polypeptides.

Non-naturally occurring variants of TR2 may be produced using art-known mutagenesis techniques, which include, but are not limited to oligonucleotide mediated mutagenesis, alanine scanning, PCR mutagenesis, site directed mutagenesis (see e.g., Carter et al., Nucl. Acids Res. 13:4331 (1986); and Zoller et al., Nucl. Acids Res. 10:6487 (1982)), cassette mutagenesis (see e.g., Wells et al., Gene 34:315 (1985)), restriction selection mutagenesis (see e.g., Wells et al., Philos. Trans. R. Soc. London SerA 317:415 (1986)).

Thus, the invention also encompasses antibodies that bind TR2 derivatives and analogs that have one or more amino acid residues deleted, added, and/or substituted to generate TR2 polypeptides that are better suited for binding activity, therapeutic activity, expression, scale up, etc., in the host cells chosen. For example, cysteine residues can be deleted or substituted with another amino acid residue in order to eliminate disulfide bridges; N-linked glycosylation sites can be altered or eliminated to achieve, for example, expression of a homogeneous product that is more easily recovered and purified from yeast hosts which are known to hyperglycosylate N-linked sites. To this end, a variety of amino acid substitutions at one or both of the first or third amino acid positions on any one or more of the glycosylation recognition sequences in the TR2 polypeptides and/or an amino acid deletion at the second position of any one or more such recognition sequences will prevent glycosylation of the TR2 at the modified tripeptide sequence (see, e.g., Miyajimo et al., EMBO J 5(6):1193-1197). Additionally, one or more of the amino acid residues of TR2 polypeptides (e.g., arginine and lysine residues) may be deleted or substituted with another residue to eliminate undesired processing by proteases such as, for example, furins or kexins.

The antibodies of the present invention also include antibodies that bind a polypeptide comprising, or alternatively, consisting of the polypeptide encoded by the deposited cDNA (the deposit having ATCC Accession Number 97059) including the leader; a polypeptide comprising, or alternatively, consisting of the mature polypeptide encoded by the deposited the cDNA minus the leader (i.e., the mature protein); a polypeptide comprising, or alternatively, consisting of the polypeptide of SEQ ID NO:2 including the leader; a polypeptide comprising, or alternatively, consisting of the polypeptide of SEQ ID NO:2 minus the amino terminal methionine; a polypeptide comprising, or alternatively, consisting of the polypeptide of SEQ ID NO:2 minus the leader; a polypeptide comprising, or alternatively, consisting of the TR2 extracellular domain; a polypeptide comprising, or alternatively, consisting of the TR2 cysteine rich domain; a polypeptide comprising, or alternatively, consisting of the TR2 transmembrane domain; a polypeptide comprising, or alternatively, consisting of the TR2 intracellular domain; a polypeptide comprising, or alternatively, consisting of soluble polypeptides comprising all or part of the extracellular and intracelluar domains but lacking the transmembrane domain; as well as polypeptides which are at least 80% identical, more preferably at least 90% or 95% identical, still more preferably at least 96%, 97%, 98% or 99% identical to the polypeptides described above (e.g., the polypeptide encoded by the deposited cDNA clone (the deposit having ATCC Accession Number 97059), the polypeptide of SEQ ID NO:2, and portions of such polypeptides with at least 30 amino acids and more preferably at least 50 amino acids.

By a polypeptide having an amino acid sequence at least, for example, 95% “identical” to a reference amino acid sequence of a TR2 polypeptide is intended that the amino acid sequence of the polypeptide is identical to the reference sequence except that the polypeptide sequence may include up to five amino acid alterations per each 100 amino acids of the reference amino acid of the TR2 polypeptide. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 90%, 95%, 96%, 97%, 98% or 99% identical to, for instance, the amino acid sequence shown in SEQ ID NO:2 or to the amino acid sequence encoded by deposited cDNA clones can be determined conventionally using known computer programs such the Bestfit program (Wisconsin Sequence Analysis Package, Version 8 for Unix, Genetics Computer Group, University Research Park, 575 Science Drive, Madison, Wis. 53711. When using Bestfit or any other sequence alignment program to determine whether a particular sequence is, for instance, 95% identical to a reference sequence according to the present invention, the parameters are set, of course, such that the percentage of identity is calculated over the full length of the reference amino acid sequence and that gaps in homology of up to 5% of the total number of amino acid residues in the reference sequence are allowed.

In a specific embodiment, the identity between a reference (query) sequence (a sequence of the present invention) and a subject sequence, also referred to as a global sequence alignment, is determined using the FASTDB computer program based on the algorithm of Brutlag et al. (Comp. App. Biosci. 6:237-245 (1990)). Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter. According to this embodiment, if the subject sequence is shorter than the query sequence due to N— or C-terminal deletions, not because of internal deletions, a manual correction is made to the results to take into consideration the fact that the FASTDB program does not account for N— and C-terminal truncations of the subject sequence when calculating global percent identity. For subject sequences truncated at the N— and C-termini, relative to the query sequence, the percent identity is corrected by calculating the number of residues of the query sequence that are N— and C-terminal of the subject sequence, which are not matched/aligned with a corresponding subject residue, as a percent of the total bases of the query sequence. A determination of whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of this embodiment. Only residues to the N— and C-termini of the subject sequence, which are not matched/aligned with the query sequence, are considered for the purposes of manually adjusting the percent identity score. That is, only query residue positions outside the farthest N— and C-terminal residues of the subject sequence. For example, a 90 amino acid residue subject sequence is aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N— and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N— or C-termini of the subject sequence which are not matched/aligned with the query. In this case the percent identity calculated by FASTDB is not manually corrected. Once again, only residue positions outside the N— and C-terminal ends of the subject sequence, as displayed in the FASTDB alignment, which are not matched/aligned with the query sequence are manually corrected for. No other manual corrections are made for the purposes of this embodiment.

The present application is also directed to antibodies that bind proteins containing polypeptides at least 90%, 95%, 96%, 97%, 98% or 99% identical to the TR2 polypeptide sequence set forth herein as n¹-m¹, and/or n²-m¹. In preferred embodiments, the application is directed to antibodies that bind proteins containing polypeptides at least 90%, 95%, 96%, 97%, 98% or 99% identical to polypeptides having the amino acid sequence of the specific TR2 N— and C-terminal deletions recited herein.

In certain preferred embodiments, antibodies of the invention bind TR2 fusion proteins as described above wherein the TR2 portion of the fusion protein are those described as n¹-m¹, and/or n²-m¹ herein.

Antibodies of the Invention May Bind Modified TR2 Polypeptides

It is specifically contmeplated that antibodies of the present invention may bind modified forms of TR2 proteins (e.g., SEQ ID NOs: 2, 4, or 6)

In specific embodiments, antibodies of the present invention bind TR2 polypeptides (such as those decribed above) including, but not limited to naturally purified TR2 polypeptides, TR2 polypeptides produced by chemical synthetic procedures, and TR2 polypeptides produced by recombinant techniques from a prokaryotic or eukaryotic host, including, for example, bacterial, yeast, higher plant, insect and mammalian cells using, for example, the recombinant compositions and methods described above. Depending upon the host employed in a recombinant production procedure, the polypeptides may be glycosylated or non-glycosylated. In addition, TR2 polypeptides may also include an initial modified methionine residue, in some cases as a result of host-mediated processes.

In addition, TR2 proteins that antibodies of the present invention may bind can be chemically synthesized using techniques known in the art (e.g., see Creighton, Proteins: Structures and Molecular Principles, W.H. Freeman & Co., N.Y. (1983), and Hunkapiller, et al., Nature 310:105-111 (1984)). For example, a peptide corresponding to a fragment of a TR2 polypeptide can be synthesized by use of a peptide synthesizer. Furthermore, if desired, nonclassical amino acids or chemical amino acid analogs can be introduced as a substitution or addition into the TR2 polypeptide sequence. Non-classical amino acids include, but are not limited to, to the D-isomers of the common amino acids, 2,4-diaminobutyric acid, a-amino isobutyric acid, 4-aminobutyric acid, Abu, 2-amino butyric acid, g-Abu, e-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, b-alanine, fluoro-amino acids, designer amino acids such as b-methyl amino acids, Ca-methyl amino acids, Na-methyl amino acids, and amino acid analogs in general. Furthermore, the amino acid can be D (dextrorotary) or L (levorotary).

The invention additionally, encompasses antibodies that bind TR2 polypeptides which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited to, specific chemical cleavage by cyanogen bromide, trypsin, chymotrypsin, papain, V8 protease, NaBH₄, acetylation, formylation, oxidation, reduction, metabolic synthesis in the presence of tunicamycin; etc.

Additional post-translational modifications to TR2 polypeptides for example, e.g., N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of procaryotic host cell expression. The polypeptides may also be modified with a detectable label, such as an enzymatic, fluorescent, isotopic or affinity label to allow for detection and isolation of the protein.

Also provided by the invention are antibodies that bind chemically modified derivatives of TR2 polypeptide which may provide additional advantages such as increased solubility, stability and circulating time of the polypeptide, or decreased immunogenicity (see U.S. Pat. No. 4,179,337). The chemical moieties for derivitization may be selected from water soluble polymers such as polyethylene glycol, ethylene glycol/propylene glycol copolymers, carboxymethylcellulose, dextran, polyvinyl alcohol and the like. The polypeptides may be modified at random positions within the molecule, or at predetermined positions within the molecule and may include one, two, three or more attached chemical moieties.

The polymer may be of any molecular weight, and may be branched or unbranched. For polyethylene glycol, the preferred molecular weight is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog). For example, the polyethylene glycol may have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 25,000, 30,000, 35,000, 40,000, 50,000, 55,000, 60,000, 65,000, 70,000, 75,000, 80,000, 85,000, 90,000, 95,000, or 100,000 kDa.

As noted above, the polyethylene glycol may have a branched structure. Branched polyethylene glycols are described, for example, in U.S. Pat. No. 5,643,575; Morpurgo et al., Appl. Biochem. Biotechnol. 56:59-72 (1996); Vorobjev et al., Nucleosides Nucleotides 18:2745-2750 (1999); and Caliceti et al., Bioconjug. Chem. 10:638-646 (1999), the disclosures of each of which are incorporated herein by reference.

The polyethylene glycol molecules (or other chemical moieties) should be attached to the protein with consideration of effects on functional or antigenic domains of the protein. There are a number of attachment methods available to those skilled in the art, e.g., EP 0 401 384, herein incorporated by reference (coupling PEG to G-CSF), see also Malik et al., Exp. Hematol. 20:1028-1035 (1992) (reporting pegylation of GM-CSF using tresyl chloride). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues, glutamic acid residues and the C-terminal amino acid residue. Sulfhydryl groups may also be used as a reactive group for attaching the polyethylene glycol molecules. Preferred for therapeutic purposes is attachment at an amino group, such as attachment at the N-terminus or lysine group.

As suggested above, polyethylene glycol may be attached to proteins via linkage to any of a number of amino acid residues. For example, polyethylene glycol can be linked to a proteins via covalent bonds to lysine, histidine, aspartic acid, glutamic acid, or cysteine residues. One or more reaction chemistries may be employed to attach polyethylene glycol to specific amino acid residues (e.g., lysine, histidine, aspartic acid, glutamic acid, or cysteine) of the protein or to more than one type of amino acid residue (e.g., lysine, histidine, aspartic acid, glutamic acid, cysteine and combinations thereof) of the protein.

One may specifically desire proteins chemically modified at the N-terminus. Using polyethylene glycol as an illustration of the present composition, one may select from a variety of polyethylene glycol molecules (by molecular weight, branching, etc.), the proportion of polyethylene glycol molecules to protein (or peptide) molecules in the reaction mix, the type of pegylation reaction to be performed, and the method of obtaining the selected N-terminally pegylated protein. The method of obtaining the N-terminally pegylated preparation (i.e., separating this moiety from other monopegylated moieties if necessary) may be by purification of the N-terminally pegylated material from a population of pegylated protein molecules. Selective proteins chemically modified at the N-terminus modification may be accomplished by reductive alkylation which exploits differential reactivity of different types of primary amino groups (lysine versus the N-terminal) available for derivatization in a particular protein. Under the appropriate reaction conditions, substantially selective derivatization of the protein at the N-terminus with a carbonyl group containing polymer is achieved.

As indicated above, pegylation of the proteins of the invention may be accomplished by any number of means. For example, polyethylene glycol may be attached to the protein either directly or by an intervening linker. Linkerless systems for attaching polyethylene glycol to proteins are described in Delgado et al., Crit. Rev. Thera. Drug Carrier Sys. 9:249-304 (1992); Francis et al., Intern. J. of Hematol. 68:1-18 (1998); U.S. Pat. No. 4,002,531; U.S. Pat. No. 5,349,052; WO 95/06058; and WO 98/32466, the disclosures of each of which are incorporated herein by reference.

One system for attaching polyethylene glycol directly to amino acid residues of proteins without an intervening linker employs tresylated MPEG, which is produced by the modification of monmethoxy polyethylene glycol (MPEG) using tresylchloride (CISO₂CH₂CF₃). Upon reaction of protein with tresylated MPEG, polyethylene glycol is directly attached to amine groups of the protein. Thus, the invention includes protein-polyethylene glycol conjugates produced by reacting proteins of the invention with a polyethylene glycol molecule having a 2,2,2-trifluoreothane sulphonyl group.

Polyethylene glycol can also be attached to proteins using a number of different intervening linkers. For example, U.S. Pat. No. 5,612,460, the entire disclosure of which is incorporated herein by reference, discloses urethane linkers for connecting polyethylene glycol to proteins. Protein-polyethylene glycol conjugates wherein the polyethylene glycol is attached to the protein by a linker can also be produced by reaction of proteins with compounds such as MPEG-succinimidylsuccinate, MPEG activated with 1,1′-carbonyldiimidazole, MPEG-2,4,5-trichloropenylcarbonate, MPEG-p-nitrophenolcarbonate, and various MPEG-succinate derivatives. A number additional polyethylene glycol derivatives and reaction chemistries for attaching polyethylene glycol to proteins are described in WO 98/32466, the entire disclosure of which is incorporated herein by reference. Pegylated protein products produced using the reaction chemistries set out herein are included within the scope of the invention.

The number of polyethylene glycol moieties attached to each TR2 polypeptide (i.e., the degree of substitution) may also vary. For example, the pegylated proteins of the invention may be linked, on average, to 1, 2, 3, 4, 5, 6, 7, 8, 9, 10, 12, 15, 17, 20, or more polyethylene glycol molecules. Similarly, the average degree of substitution within ranges such as 1-3, 24, 3-5, 4-6, 5-7, 6-8, 7-9, 8-10, 9-11, 10-12, 11-13, 12-14, 13-15, 14-16, 15-17, 16-18, 17-19, or 18-20 polyethylene glycol moieties per protein molecule. Methods for determining the degree of substitution are discussed, for example, in Delgado et al., Crit. Rev. Thera. Drug Carrier Sys. 9:249-304 (1992).

As mentioned the antibodies of the present invention may bind TR2 polypeptides that are modified by either natural processes, such as posttranslational processing, or by chemical modification techniques which are well known in the art. It will be appreciated that the same type of modification may be present in the same or varying degrees at several sites in a given TR2 polypeptide. TR2 polypeptides may be branched, for example, as a result of ubiquitination, and they may be cyclic, with or without branching. Cyclic, branched, and branched cyclic TR2 polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, PROTEINS—STRUCTURE AND MOLECULAR PROPERTIES, 2nd Ed., T. E. Creighton, W. H. Freeman and Company, New York (1993); POSTTRANSLATIONAL COVALENT MODIFICATION OF PROTEINS, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992)).

In one embodiment, the invention provides antibodies (e.g., antibodies comprising two heavy chains and two light chains linked together by disulfide bridges) that specifically bind a TR2 polypeptide (e.g., SEQ ID NOs: 2, 4, or 6) or fragments or variants thereof, wherein the amino acid sequence of the heavy chain and the amino acid sequence of the light chain are the same as the amino acid sequence of a heavy chain and a light chain expressed by one or more cell lines referred to in Table 1. In another embodiment, the invention provides antibodies (each consisting of two heavy chains and two light chains linked together by disulfide bridges to form an antibody) that specifically bind a TR2 polypeptide (e.g., SEQ ID NOs: 2, 4, or 6) or fragments or variants thereof, wherein the amino acid sequence of the heavy chain or the amino acid sequence of the light chain are the same as the amino acid sequence of a heavy chain or a light chain expressed by one or more cell lines referred to in Table 1. Specific binding to TR2 polypeptides may be determined by immunoassays known in the art or described herein for assaying specific antibody-antigen binding. Molecules comprising, or alternatively consisting of, fragments or variants of these antibodies that specifically bind to TR2 are also encompassed by the invention, as are nucleic acid molecules encoding these antibodies molecules, fragments and/or variants (e.g., SEQ ID NO:7).

Anti-TR2 Antibodies

In one embodiment of the present invention, antibodies that specifically bind to TR2 or a fragment or variant thereof, comprise a polypeptide having the amino acid sequence of any one of the heavy chains of an antibody expressed by at least one of the cell lines referred to in Table 1 and/or any one of the light chains of an antibody expressed by at least one of the cell lines referred to in Table 1.

In another embodiment of the present invention, antibodies that specifically bind to TR2 or a fragment or variant thereof, comprise a polypeptide having the amino acid sequence of any one of the VH domains of at least one of the scFvs referred to in Table 1 and/or any one of the VL domains of at least one of the scFvs referred to in Table 1. In preferred embodiments, antibodies of the present invention comprise the amino acid sequence of a VH domain and VL domain of the scFvs referred to in Table 1. Molecules comprising, or alternatively consisting of, antibody fragments or variants of the VH and/or VL domains of at least one of the scFvs referred to in Table 1 that specifically bind to TR2 are also encompassed by the invention, as are nucleic acid molecules encoding these VH and VL domains, molecules, fragments and/or variants.

The present invention also provides antibodies that specifically bind to a polypeptide, or polypeptide fragment or variant of TR2, wherein said antibodies comprise, or alternatively consist of, a polypeptide having an amino acid sequence of any one, two, three, or more of the VH CDRs contained in a VH domain of one or more scFvs referred to in Table 1. In particular, the invention provides antibodies that specifically bind TR2, comprising, or alternatively consisting of, a polypeptide having the amino acid sequence of a VH CDR 1 contained in a VH domain of one or more scFvs referred to in Table 1. In another embodiment, antibodies that specifically bind TR2, comprise, or alternatively consist of, a polypeptide having the amino acid sequence of a VH CDR2 contained in a VH domain of one or more scFvs referred to in Table 1. In a preferred embodiment, antibodies that specifically bind TR2, comprise, or alternatively consist of a polypeptide having the amino acid sequence of a VH CDR3 contained in a VH domain of one or more scFvs referred to in Table 1. Molecules comprising, or alternatively consisting of, these antibodies, or antibody fragments or variants thereof, that specifically bind to TR2 or a fragment or variant thereof are also encompassed by the invention, as are nucleic acid molecules encoding these antibodies, molecules, fragments and/or variants (e.g., SEQ ID NO:7).

The present invention also provides antibodies that specifically bind to a polypeptide, or polypeptide fragment or variant of TR2, wherein said antibodies comprise, or alternatively consist of, a polypeptide having an amino acid sequence of any one, two, three, or more of the VL CDRs contained in a VL domain of one or more scFvs referred to in Table 1. In particular, the invention provides antibodies that specifically bind TR2, comprising, or alternatively consisting of, a polypeptide having the amino acid sequence of a VL CDR1 contained in a VL domain of one or more scFvs referred to in Table 1. In another embodiment, antibodies that specifically bind TR2, comprise, or alternatively consist of, a polypeptide having the amino acid sequence of a VL CDR2 contained in a VL domain of one or more scFvs referred to in Table 1. In a preferred embodiment, antibodies that specifically bind TR2, comprise, or alternatively consist of a polypeptide having the amino acid sequence of a VL CDR3 contained in a VL domain of one or more scFvs referred to in Table 1. Molecules comprising, or alternatively consisting of, these antibodies, or antibody fragments or variants thereof, that specifically bind to TR2 or a fragment or variant thereof are also encompassed by the invention, as are nucleic acid molecules encoding these antibodies, molecules, fragments and/or variants (e.g., SEQ ID NO:7).

The present invention also provides antibodies (including molecules comprising, or alternatively consisting of, antibody fragments or variants) that specifically bind to a TR2 polypeptide or polypeptide fragment or variant of TR2, wherein said antibodies comprise, or alternatively consist of, one, two, three, or more VH CDRs and one, two, three or more VL CDRs, as contained in a VH domain or VL domain of one or more scFvs referred to in Table 1. In particular, the invention provides for antibodies that specifically bind to a polypeptide or polypeptide fragment or variant of TR2, wherein said antibodies comprise, or alternatively consist of, a VH CDR1 and a VL CDR1, a VH CDR1 and a VL CDR2, a VH CDR1 and a VL CDR3, a VH CDR2 and a VL CDR1, VH CDR2 and VL CDR2, a VH CDR2 and a VL CDR3, a VH CDR3 and a VH CDR1, a VH CDR3 and a VL CDR2, a VH CDR3 and a VL CDR3, or any combination thereof, of the VH CDRs and VL CDRs contained in a VH domain or VL domain of one or more scFvs referred to in Table 1. In a preferred embodiment, one or more of these combinations are from the same scFv as disclosed in Table 1. Molecules comprising, or alternatively consisting of, fragments or variants of these antibodies, that specifically bind to TR2 are also encompassed by the invention, as are nucleic acid molecules encoding these antibodies, molecules, fragments or variants (e.g., SEQ ID NO:7).

Nucleic Acid Molecules Encoding Anti-TR2 Antibodies

The present invention also provides for nucleic acid molecules, generally isolated, encoding an antibody of the invention (including molecules comprising, or alternatively consisting of, antibody fragments or variants thereof). In specific embodiments, the nucleic acid molecules encoding an antibody of the invention comprise, or alternatively consist of SEQ ID NO:7 or fragments or variants thereof.

In a specific embodiment, a nucleic acid molecule of the invention encodes an antibody (including molecules comprising, or alternatively consisting of, antibody fragments or variants thereof), comprising, or alternatively consisting of, a VH domain having an amino acid sequence of any one of the VH domains of at least one of the scFvs referred to in Table 1 and a VL domain having an amino acid sequence of VL domain of at least one of the scFvs referred to in Table 1. In another embodiment, a nucleic acid molecule of the invention encodes an antibody (including molecules comprising, or alternatively consisting of, antibody fragments or variants thereof), comprising, or alternatively consisting of, a VH domain having an amino acid sequence of any one of the VH domains of at least one of the scFvs referred to in Table 1 or a VL domain having an amino acid sequence of a VL domain of at least one of the scFvs referred to in Table 1.

The present invention also provides antibodies that comprise, or alternatively consist of, variants (including derivatives) of the antibody molecules (e.g., the VH domains and/or VL domains) described herein, which antibodies specifically bind to a TR2 polypeptide or a fragment or variant thereof. Standard techniques known to those of skill in the art can be used to introduce mutations in the nucleotide sequence encoding a molecule of the invention, including, for example, site-directed mutagenesis and PCR-mediated mutagenesis which result in amino acid substitutions. Preferably, the variants (including derivatives) encode less than 50 amino acid substitutions, less than 40 amino acid subsitutions, less than 30 amino acid substitutions, less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the reference VH domain, VHCDR1, VHCDR2, VHCDR3, VL domain, VLCDR1, VLCDR2, or VLCDR3. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity (e.g., the ability to bind TR2).

For example, it is possible to introduce mutations only in framework regions or only in CDR regions of an antibody molecule. Introduced mutations may be silent or neutral missense mutations, i.e., have no, or little, effect on an antibody's ability to bind antigen. These types of mutations may be useful to optimize codon usage, or improve a hybriodma's antibody production. Alternatively, non-neutral missense mutations may alter an antibody's ability to bind antigen. The location of most silent and neutral missense mutations is likely to be in the framework regions, while the location of most non-neutral missense mutations is likely to be in CDR, though this is not an absolute requirement. One of skill in the art would be able to design and test mutant molecules with desired properties such as no alteration in antigen binding activity or alteration in binding activity (e.g, improvements in antigen binding activity or change in antibody specificity). Following mutagenesis, the encoded protein may routinely be expressed and the functional and/or biological activity of the encoded protein, (e.g., ability to specifically bind TR2) can be determined using techniques described herein or by routinely modifying techniques known in the art.

In a specific embodiment, an antibody of the invention (including a molecule comprising, or alternatively consisting of, an antibody fragment or variant thereof), that specifically binds TR2 polypeptides or fragments or variants thereof, comprises, or alternatively consists of, an amino acid sequence encoded by a nucleotide sequence that hybridizes to a nucleotide sequence that is complementary to that encoding one of the VH or VL domains of one or more scFvs referred to in Table 1. under stringent conditions, e.g., hybridization to filter-bound DNA in 6× sodium chloride/sodium citrate (SSC) at about 45° C. followed by one or more washes in 0.2×SSC/0. 1% SDS at about 50-65° C., under highly stringent conditions, e.g., hybridization to filter-bound nucleic acid in 6×SSC at about 45° C. followed by one or more washes in 0. 1×SSC/0.2% SDS at about 68° C., or under other stringent hybridization conditions which are known to those of skill in the art (see, for example, Ausubel, F. M. et al., eds. , 1989, Current Protocols in Molecular Biology, Vol. I, Green Publishing Associates, Inc. and John Wiley & Sons, Inc., New York at pages 6.3.1-6.3.6 and 2.10.3). Nucleic acid molecules encoding these antibodies are also encompassed by the invention.

It is well known within the art that polypeptides, or fragments or variants thereof, with similar amino acid sequences often have similar structure and many of the same biological activities. Thus, in one embodiment, an antibody (including a molecule comprising, or alternatively consisting of, an antibody fragment or variant thereof), that specifically binds to a TR2 polypeptide or fragments or variants of a TR2 polypeptide, comprises, or alternatively consists of, a VH domain having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical, to the amino acid sequence of a VH domain of at least one of the scFvs referred to in Table 1.

In another embodiment, an antibody (including a molecule comprising, or alternatively consisting of, an antibody fragment or variant thereof), that specifically binds to a TR2 polypeptide or fragments or variants of a TR2 polypeptide, comprises, or alternatively consists of, a VL domain having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical, to the amino acid sequence of a VL domain of at least one of the scFvs referred to in Table 1.

Methods of Producing Antibodies

Antibodies in accordance with the invention are preferably prepared utilizing a phage scFv display library. Technologies utilized for achieving the same are disclosed in the patents, applications, and references disclosed herein.

In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of lymphoid tissues) or synthetic cDNA libraries. The DNA encoding the VH and VL domains are joined together by an scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to an antigen of interest (i.e., a TR2 polypeptide or a fragment thereof) can be selected or identified with antigen, e.g., using labeled antigen or antigen bound or captured to a solid surface or bead. Examples of phage display methods that can be used to make the antibodies of the present invention include, but are not limited to, those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280(1994); PCT application No. PCT/GB91/01 134; PCT publications WO 90/02809; WO 91/10737; WO 92/01047; WO 92/18719; WO 93/1 1236; WO 95/15982; WO 95/20401; WO97/13844; and U.S. Pat. Nos. 5,698,426; 5,223,409; 5,403,484; 5,580,717; 5,427,908; 5,750,753; 5,821,047; 5,571,698; 5,427,908; 5,516,717; 5,780,225; 5,658,727; 5,735,743 and 5,969,108; each of which is incorporated herein by reference in its entirety.

For some uses, such as for in vitro affinity maturation of an antibody of the invention, it may be useful to express the VH and VL domains of one or more scFvs referred to in Table 1 as single schain antibodies or Fab fragments in a phage display library. For example, the cDNAs encoding the VH and VL domains of the scFvs referred to in Table 1 may be expressed in all possible combinations using a phage display library, allowing for the selection of VH/VL combinations that bind a TR2 polypeptides with preferred binding characteristics such as improved affinity or improved off rates. Additionally, VH and VL segments—the CDR regions of the VH and VL domains of the scFvs referred to in Table 1, in particular, may be mutated in vitro. Expression of VH and VL domains with “mutant” CDRs in a phage display library allows for the selection of VH/VL combinations that bind a TR2 polypeptides with preferred binding characteristics such as improved affinity or improved off rates.

Additional Methods of Producing Antibodies

Antibodies of the invention (including antibody fragments or variants) can be produced by any method known in the art. For example, it will be appreciated that antibodies in accordance with the present invention can be expressed in cell lines, including, but not limited to, myeloma cell lines and hybridoma cell lines. Sequences encoding the cDNAs or genomic clones for the particular antibodies can be used for transformation of a suitable mammalian or nonmammalian host cells or to generate phage display libraries, for example. Additionally, polypeptide antibodies of the invention may be chemically synthesized or produced through the use of recombinant expression systems.

One way to produce the antibodies of the invention would be to clone the VH and/or VL domains of the scFvs referred to in Table 1. In order to isolate the VH and VL domains from bacteria transfected with a vector containing the scFv, PCR primers complementary to VH or VL nucleotide sequences (See Example 2), may be used to amplify the VH and VL sequences. The PCR products may then be cloned using vectors, for example, which have a PCR product cloning site consisting of a 5′ and 3′ single T nucleotide overhang, that is complementary to the overhanging single adenine nucleotide added onto the 5′ and 3′ end of PCR products by many DNA polymerases used for PCR reactions. The VH and VL domains can then be sequenced using conventional methods known in the art. Alternatively, the VH and VL domains may be amplified using vector specific primers designed to amplify the entire scFv, (i.e. the VH doamin, linker and VL domain.)

The cloned VH and VL genes may be placed into one or more suitable expression vectors. By way of non-limiting example, PCR primers including VH or VL nucleotide sequences, a restriction site, and a flanking sequence to protect the restriction site may be used to amplify the VH or VL sequences. Utilizing cloning techniques known to those of skill in the art, the PCR amplified VH domains may be cloned into vectors expressing the appropriate immunoglobulin constant region, e.g., the human IgG1 or IgG4 constant region for VH domains, and the human kappa or lambda constant regions for kappa and lambda VL domains, respectively. Preferably, the vectors for expressing the VH or VL domains comprise a promoter suitable to direct expression of the heavy and light chains in the chosen expression system, a secretion signal, a cloning site for the immunoglobulin variable domain, immunoglobulin constant domains, and a selection marker such as neomycin. The VH and VL domains may also be cloned into a single vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art (See, for example, Guo et al., J. Clin. Endocrinol. Metab. 82:925-31 (1997), and Ames et al., J. Immunol. Methods 184:177-86 (1995) which are herein incorporated in their entireties by reference).

The invention provides polynucleotides comprising, or alternatively consisting of, a nucleotide sequence encoding an antibody of the invention (including molecules comprising, or alternatively consisting of, antibody fragments or variants thereof). The invention also encompasses polynucleotides that hybridize under high stringency, or alternatively, under intermediate or lower stringency hybridization conditions, e.g., as defined supra, to polynucleotides complementary to nucleic acids having a polynucleotide sequence that encodes an antibody of the invention or a fragment or variant thereof.

The polynucleotides may be obtained, and the nucleotide sequence of the polynucleotides determined, by any method known in the art. If the amino acid sequences of the VH domains, VL domains and CDRs thereof, are known, nucleotide sequences encoding these antibodies can be determined using methods well known in the art, i.e., the nucleotide codons known to encode the particular amino acids are assembled in such a way to generate a nucleic acid that encodes the antibody, of the invention. Such a polynucleotide encoding the antibody may be assembled from chemically synthesized oligonucleotides (e.g., as described in Kutmeier et al., BioTechniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody (including molecules comprising, or alternatively consisting of, antibody fragments or variants thereof) may be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, preferably poly A+ RNA, isolated from, any tissue or cells expressing the antibody, such as hybridoma cells or Epstein Barr virus transformed B cell lines that express an antibody of the invention) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence of the antibody (including molecules comprising, or alternatively consisting of, antibody fragments or variants thereof) is determined, the nucleotide sequence of the antibody may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties), to generate antibodies having a different amino acid sequence, for example to create amino acid substitutions, deletions, and/or insertions.

In a specific embodiment, VH and VL domains of one or more scFvs referred to in Table 1, or fragments or variants thereof, are inserted within antibody framework regions using recombinant DNA techniques known in the art. In a specific embodiment, one, two, three, four, five, six, or more of the CDRs of VH and/or VL domains of one or more scFvs referred to in Table 1, or fragments or variants thereof, is inserted within antibody framework regions using recombinant DNA techniques known in the art. The framework regions may be naturally occurring or consensus antibody framework regions, and preferably human antibody framework regions (see, e.g., Chothia et al., J. Mol. Biol. 278: 457-479 (1998) for a listing of human antibody framework regions, the contents of which are hereby incorporated by reference in its entirety). Preferably, the polynucleotides generated by the combination of the antibody framework regions and CDRs encode an antibody (including molecules comprising, or alternatively consisting of, antibody fragments or variants thereof) that specifically binds to TR2. Preferably, as discussed supra, polynucleotides encoding variants of antibodies or antibody fragments having one or more amino acid substitutions may be made within the framework regions, and, preferably, the amino acid substitutions do not significantly alter binding of the antibody to its antigen. Additionally, such methods may be used to make amino acid substitutions or deletions of one or more variable region cysteine residues participating in an intrachain disulfide bond to generate antibody molecules, or antibody fragments or variants, lacking one or more intrachain disulfide bonds. Other alterations to the polynucleotide are encompassed by the present invention and fall within the ordinary skill of the art.

Xenomouse™ Technology

The ability to clone and reconstruct megabase-sized human loci in YACs and to introduce them into the mouse germline provides a powerful approach to elucidating the functional components of very large or crudely mapped loci as well as generating useful models of human disease. Furthermore, the utilization of such technology for substitution of mouse loci with their human equivalents could provide unique insights into the expression and regulation of human gene products during development, their communication with other systems, and their involvement in disease induction and progression.

An important practical application of such a strategy is the “humanization” of the mouse humoral immune system. Introduction of human immunoglobulin (Ig) loci into mice in which the endogenous Ig genes have been inactivated offers the opportunity to study the mechanisms underlying programmed expression and assembly of antibodies as well as their role in B cell development. Furthermore, such a strategy could provide an ideal source for production of fully human monoclonal antibodies (Mabs) an important milestone towards fulfilling the promise of antibody therapy in human disease.

Fully human antibodies are expected to minimize the immunogenic and allergic responses intrinsic to mouse or mouse-derivatized Monoclonal antibodies and thus to increase the efficacy and safety of the administered antibodies. The use of fully human antibodies can be expected to provide a substantial advantage in the treatment of chronic and recurring human diseases, such as cancer, which require repeated antibody administrations.

One approach towards this goal was to engineer mouse strains deficient in mouse antibody production with large fragments of the human Ig loci in anticipation that such mice would produce a large repertoire of human antibodies in the absence of mouse antibodies. Large human Ig fragments would preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains should yield high affinity antibodies against any antigen of interest, including human antigens. Using the hybridoma technology, antigen-specific human Monoclonal antibodies with the desired specificity could be readily produced and selected.

This general strategy was demonstrated in connection with the generation of the first XenoMouse™ strains as published in 1994. See Green et al. Nature Genetics 7:13-21 (1994). The XenoMouse™ strains were engineered with yeast artificial chromosomes (YACS) containing 245 kb and10 190 kb-sized germline configuration fragments of the human heavy chain locus and kappa light chain locus, respectively, which contained core variable and constant region sequences. Id. The human Ig containing YACs proved to be compatible with the mouse system for both rearrangement and expression of antibodies and were capable of substituting for the inactivated mouse Ig genes. This was demonstrated by their ability to induce B-cell development, to produce an adult-like human repertoire of fully human antibodies, and to generate antigen-specific human monoclonal antibodies. These results also suggested that introduction of larger portions of the human Ig loci containing greater numbers of V genes, additional regulatory elements, and human Ig constant regions might recapitulate substantially the full repertoire that is characteristic of the human humoral response to infection and immunization. The work of Green et al. was recently extended to the introduction of greater than approximately 80% of the human antibody repertoire through introduction of megabase sized, germline configuration YAC fragments of the human heavy chain loci and kappa light chain loci, respectively, to produce XenoMouse™ mice. See Mendez et al. Nature Genetics 15:146-156 (1997), Green and Jakobovits J Exp. Med. 188:483-495 (1998), Green, Journal of Immunological Methods 231:11-23 (1999) and U.S. patent application Ser. No. 08/759,620, filed Dec. 3, 1996, the disclosures of which are hereby incorporated by reference.

Such approach is further discussed and delineated in U.S. patent application Ser. No. 07/466,008, filed Jan. 12, 1990, Ser. No. 07/710,515, filed Nov. 8, 1990, Ser. No. 07/919,297, filed Jul. 24, 1992, Ser. No. 07/922,649, filed Jul. 30, 1992, filed Ser. No. 08/031,801, filed Mar. 15, 1993, Ser. No. 08/112,848, filed Aug. 27, 1993, Ser. No. 08/234,145, filed Apr. 28, 1994, Ser. No. 08/376,279, filed Jan. 20, 1995, Ser. No. 08/430,938, Apr. 27, 1995, Ser. No. 0-8/464,584, filed Jun. 5, 1995, Ser. No. 08/464,582, filed Jun. 5, 1995, Ser. No. 08/471,191, filed Jun. 5, 1995, Ser. No. 08/462,837, filed Jun. 5, 1995, Ser. No. 08/486,853, filed Jun. 5, 1995, Ser. No. 08/486,857, filed Jun. 5, 1995, Ser. No. 08/486,859, filed Jun. 5, 1995, Ser. No. 08/462,513, filed Jun. 5, 1995, Ser. No. 08/724,752, filed Oct. 2, 1996, and Ser. No. 08/759,620, filed Dec. 3, 1996. See also Mendez et al. Nature Genetics 15:146-156 (1997) and Green and Jakobovits J Exp. Med. 188:483 495 (1998). See also European Patent No., EP 0 463 151 B1, grant published Jun. 12, 1996, International Patent Application No., WO 94/02602, published Feb. 3, 1994, International Patent Application No., WO 96/34096, published Oct. 31, 1996, and WO 98/24893, published Jun. 11, 1998. The disclosures of each of the above-cited patents, applications, and references are hereby incorporated by reference in their entirety.

Human anti-mouse antibody (HAMA) responses have led the industry to prepare chimeric or otherwise humanized antibodies. While chimeric antibodies have a human constant region and a murine variable region, it is expected that certain human anti-chimeric antibody (HACA) responses will be observed, particularly in chronic or multi-dose utilizations of the antibody. Thus, it would be desirable to provide fully human antibodies against TR2 polypeptides in order to vitiate concerns and/or effects of HAMA or HACA responses.

Monoclonal antibodies specific for TR2 polypeptides may be prepared using hybridoma technology. (Kohler et al., Nature 256:495 (1975); Kohler et al., Eur. J. Immunol. 6:511 (1976); Kohler et al., Eur. J. Immunol. 6:292 (1976); Hammerling et al., in: Monoclonal Antibodies and T-Cell Hybridomas, Elsevier, N.Y., pp. 571-681 (1981)). Briefly, XenoMouse™ mice may be immunized with TR2 polypeptides. After immunization, the splenocytes of such mice may be extracted and fused with a suitable myeloma cell line. Any suitable myeloma cell line may be employed in accordance with the present invention; however, it is preferable to employ the parent myeloma cell line (SP2O), available from the ATCC. After fusion, the resulting hybridoma cells are selectively maintained in HAT medium, and then cloned by limiting dilution as described by Wands et al. (Gastroenterology 80:225-232 (1981)). The hybridoma cells obtained through such a selection are then assayed to identify clones which secrete antibodies capable of binding TR2 polypetides.

For some uses, including in vivo use of antibodies in humans and in vitro detection assays, it may be preferable to use human or chimeric antibodies. Completely human antibodies are particularly desirable for therapeutic treatment of human patients. See also, U.S. Pat. Nos. 4,444,887 and 4,716,111; and PCT publications WO 98/46645, WO 98/50435, WO 98/24893, WO98/16654, WO 96/34096, WO 96/35735, and WO 91/10741; each of which is incorporated herein by reference in its entirety. In a specific embodiment, antibodies of the present invention comprise one or more VH and VL domains of the invention and constant regions from another immunoglobulin molecule, preferably a human immunoglobulin molecule. In a specific embodiment, antibodies of the present invention comprise one or more CDRs corresponding to the VH and VL domains of the invention and framework regions from another immunoglobulin molecule, preferably a human immunoglobulin molecule. In other embodiments, an antibody of the present invention comprises one, two, three, four, five, six or more VL CDRs or VH CDRs corresponding to one or more of the VH or VL domains of one or more scFvs referred to in Table 1, or fragments or variants thereof, and framework regions (and, optionally one or more CDRs not present in the antibodies expressed by scFvs referred to in Table 1) from a human immunoglobulin molecule. In a preferred embodiment, an antibody of the present invention comprises a VH CDR3, VL CDR3, or both, corresponding to the same scFv, or different scFvs selected from the scFvs referred to in Table 1, or fragments or variants thereof, and framework regions from a human immunoglobulin.

A chimeric antibody is a molecule in which different portions of the antibody are derived from different immunoglobulin molecules such as antibodies having a human variable region and a non-human (e.g., murine) immunoglobulin constant region or vice versa. Methods for producing chimeric antibodies are known in the art. See e.g., Morrison, Science 229:1202 (1985); Oi et al., BioTechniques 4:214 (1986); Gillies et al., J. Immunol. Methods 125:191-202 (1989); U.S. Pat. Nos. 5,807,715; 4,816,567; and 4,816,397, which are incorporated herein by reference in their entirety. Chimeric antibodies comprising one or more CDRs from human species and framework regions from a non-human immunoglobulin molecule (e.g., framework regions from a murine, canine or feline immunoglobulin molecule) (or vice versa) can be produced using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska et al., PNAS 91:969-973 (1994)), and chain shuffling (U.S. Pat. No. 5,565,352). In a preferred embodiment, chimeric antibodies comprise a human CDR3 having an amino acid sequence of any one of the VH CDR3s or VL CDR3s of a VH or VL domain of one or more of the scFvs referred to in Table 1, or a variant thereof, and non-human framework regions or human framework regions different from those of the frameworks in the corresponding scFv disclosed in Table 1. Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 352:323 (1988), which are incorporated herein by reference in their entireties.)

Intrabodies are antibodies, often scFvs, that are expressed from a recombinant nucleic acid molecule and engineered to be retained intracellularly (e.g., retained in the cytoplasm, endoplasmic reticulum, or periplasm). Intrabodies may be used, for example, to ablate the function of a protein to which the intrabody binds. The expression of intrabodies may also be regulated through the use of inducible promoters in the nucleic acid expression vector comprising the intrabody. Intrabodies of the invention can be produced using methods known in the art, such as those disclosed and reviewed in Chen et al., Hum. Gene Ther. 5:595-601 (1994); Marasco, W. A., Gene Ther. 4:11-15 (1997); Rondon and Marasco, Annu. Rev. Microbiol. 51:257-283 (1997); Proba et al., J. Mol. Biol. 275:245-253 (1998); Cohen et al., Oncogene 17:2445-2456 (1998); Ohage and Steipe, J. Mol. Biol. 291:1119-1128 (1999); Ohage et al., J. Mol. Biol. 291:1129-1134 (1999); Wirtz and Steipe, Protein Sci. 8:2245-2250 (1999); Zhu et al., J. Immunol. Methods 231:207-222 (1999); and references cited therein.

Recombinant expression of an antibody of the invention (including antibody fragments or variants thereof (e.g., a heavy or light chain of an antibody of the invention), requires construction of an expression vector(s) containing a polynucleotide that encodes the antibody. Once a polynucleotide encoding an antibody molecule (e.g., a whole antibody, a heavy or light chain of an antibody, or portion thereof (preferably, but not necessarily, containing the heavy or light chain variable domain)), of the invention has been obtained, the vector(s) for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention (e.g., a whole antibody, a heavy or light chain of an antibody, a heavy or light chain variable domain of an antibody, or a portion thereof, or a heavy or light chain CDR, a single chain Fv, or fragments or variants thereof), operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464, the contents of each of which are hereby incorporated by reference in its entirety) and the variable domain of the antibody may be cloned into such a vector for expression of the entire heavy chain, the entire light chain, or both the entire heavy and light chains.

The expression vector(s) is(are) transferred to a host cell by conventional techniques and the transfected cells are then cultured by conventional techniques to produce an antibody of the invention. Thus, the invention includes host cells containing polynucleotide(s) encoding an antibody of the invention (e.g., whole antibody, a heavy or light chain thereof, or portion thereof, or a single chain antibody, or a fragment or variant thereof), operably linked to a heterologous promoter. In preferred embodiments, for the expression of entire antibody molecules, vectors encoding both the heavy and light chains may be co-expressed in the host cell for expression of the entire immunoglobulin molecule, as detailed below.

A variety of host-expression vector systems may be utilized to express the antibody molecules of the invention. Such host-expression systems represent vehicles by which the coding sequences of interest may be produced and subsequently purified, but also represent cells which may, when transformed or transfected with the appropriate nucleotide coding sequences, express an antibody molecule of the invention in situ. These include, but are not limited to, bacteriophage particles engineered to express antibody fragments or variants teherof (single chain antibodies), microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3, NS0 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., Gene 45:101 (1986); Cockett et al., Bio/Technology 8:2 (1990); Bebbington et al., Bio/Techniques 10:169 (1992); Keen and Hale, Cytotechnology 18:207 (1996)). These references are incorporated in their entirities by refernce herein.

In bacterial systems, a number of expression vectors may be advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited to, the E. coli expression vector pUR278 (Ruther et al., EMBO 1. 2:1791 (1983)), in which the antibody coding sequence may be ligated individually into the vector in frame with the lac Z coding region so that a fusion protein is produced; pIN vectors (Inouye & Inouye, Nucleic Acids Res. 13:3101-3109 (1985); Van Heeke & Schuster, J. Biol. Chem. 24:5503-5509 (1989)); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) may be used as a vector to express foreign genes. The virus grows in Spodoptera frugiperda cells. Antibody coding sequences may be cloned individually into non-essential regions (for example, the polyhedrin gene) of the virus and placed under control of an AcNPV promoter (for example, the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, Proc. Natl. Acad. Sci. USA 8 1:355-359 (1984)). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., Methods in Enzymol. 153:51-544 (1987)).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include, but are not limited to, CHO, VERY, BHK, Hela, COS, NSO, MDCK, 293, 3T3, W138, and in particular, breast cancer cell lines such as, for example, BT483, Hs578T, HTB2, BT20 and T47D, and normal mammary gland cell line such as, for example, CRL7030 and HsS78Bst.

For long-term, high-yield production of recombinant proteins, stable expression is preferred. For example, cell lines which stably express the antibody may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compositions that interact directly or indirectly with the antibody molecule.

A number of selection systems may be used, including but not limited to, the herpes simplex virus thymidine kinase (Wigler et al., Cell 11:223 (1977)), hypoxanthine-guanine phosphoribosyltransferase (Szybalska & Szybalski, Proc. Natl. Acad. Sci. USA 48:202 (1992)), and adenine phosphoribosyltransferase (Lowy et al., Cell 22:8 17 (1980)) genes can be employed in tk-, hgprt- or aprt- cells, respectively. Also, antimetabolite resistance can be used as the basis of selection for the following genes: dhfr, which confers resistance to methotrexate (Wigler et al., Natl. Acad. Sci. USA 77:357 (1980); O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 (Clinical Pharmacy 12:488-505; Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62: 191-217 (1993); TIB TECH 11(5):155-2 15 (May, 1993)); and hygro, which confers resistance to hygromycin (Santerre et al., Gene 30:147 (1984)). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., J. Mol. Biol. 150:1 (1981), which are incorporated by reference herein in their entireties.

The expression levels of an antibody molecule can be increased by vector amplification (for a review, see Bebbington and Hentschel, “The use of vectors based on gene amplification for the expression of cloned genes in mammalian cells” in DNA Cloning, Vol.3. (Academic Press, New York, 1987)). When a marker in the vector system expressing antibody is amplifiable, increase in the level of inhibitor present in culture of host cell will increase the number of copies of the marker gene. Since the amplified region is associated with the coding sequence of the antibody, production of the antibody will also increase (Crouse et al., Mol. Cell. Biol. 3:257 (1983)).

Vectors which use glutamine synthase (GS) or DHFR as the selectable markers can be amplified in the presence of the drugs methionine sulphoximine or methotrexate, respectively. An advantage of glutamine synthase based vectors are the availabilty of cell lines (e.g., the murine myeloma cell line, NS0) which are glutamine synthase negative. Glutamine synthase expression systems can also function in glutamine synthase expressing cells (e.g. Chinese Hamster Ovary (CHO) cells) by providing additional inhibitor to prevent the functioning of the endogenous gene. A glutamine synthase expression system and components thereof are detailed in PCT publications: WO87/04462; WO86/05807; WO89/01036; WO89/10404; and WO91/06657 which are incorporated in their entireties by reference herein. Additionally, glutamine synthase expression vectors that may be used according to the present invention are commercially available from suplliers, including, for example Lonza Biologics, Inc. (Portsmouth, N.H.). Expression and production of monoclonal antibodies using a GS expression system in murine myeloma cells is described in Bebbington et al., Bioltechnology 10:169(1992) and in Biblia and Robinson Biotechnol. Prog. 11:1 (1995) which are incorporated in their entirities by reference herein.

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain is preferably placed before the heavy chain to avoid an excess of toxic free heavy chain (Proudfoot, Nature 322:52 (1986); Kohler, Proc. Natl. Acad. Sci. USA 77:2 197 (1980)). The coding sequences for the heavy and light chains may comprise cDNA or genomic DNA.

Once an antibody molecule of the invention (including molecules comprising, or alternatively consisting of, antibody fragments or variants thereof) has been chemically synthesized or recombinantly expressed, it may be purified by any method known in the art for purification of an immunoglobulin molecule, or more generally, a protein molecule, such as, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies of the present invention may be fused to heterologous polypeptide sequences described herein or otherwise known in the art, to facilitate purification.

Antibodies of the present invention include naturally purified products, products of chemical synthetic procedures, and products produced by recombinant techniques from a prokaryotic or eukaryotic host, including, for example, bacterial, yeast, higher plant, insect and mammalian cells. Depending upon the host employed in a recombinant production procedure, the antibodies of the present invention may be glycosylated or may be non-glycosylated. In addition, antibodies of the invention may also include an initial modified methionine residue, in some cases as a result of host-mediated processes.

Antibodies of the invention can be chemically synthesized using techniques known in the art (e.g., see Creighton, 1983, Proteins: Structures and Molecular Principles, W.H. Freeman & Co., N.Y., and Hunkapiller, M., et al., 1984, Nature 310:105-111). For example, a peptide corresponding to a fragment of an antibody of the invention can be synthesized by use of a peptide synthesizer. Furthermore, if desired, nonclassical amino acids or chemical amino acid analogs can be introduced as a substitution or addition into the antibody polypeptide sequence. Non-classical amino acids include, but are not limited to, to the D-isomers of the common amino acids, 2,4-diaminobutyric acid, a-amino isobutyric acid, 4-aminobutyric acid, Abu, 2-amino butyric acid, g-Abu, e-Ahx, 6-amino hexanoic acid, Aib, 2-amino isobutyric acid, 3-amino propionic acid, omithine, norleucine, norvaline, hydroxyproline, sarcosine, citrulline, homocitrulline, cysteic acid, t-butylglycine, t-butylalanine, phenylglycine, cyclohexylalanine, b-alanine, fluoro-amino acids, designer amino acids such as b-methyl amino acids, Ca-methyl amino acids, Na-methyl amino acids, and amino acid analogs in general. Furthermore, the amino acid can be D (dextrorotary) or L (levorotary).

The invention encompasses antibodies which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited, to specific chemical cleavage by cyanogen bromide, trypsin, chymotrypsin, papain, V8 protease, NaBH4, acetylation, formylation, oxidation, reduction, metabolic synthesis in the presence of tunicamycin, etc.

Additional post-translational modifications encompassed by the invention include, for example, e.g., N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of procaryotic host cell expression. The antibodies may also be modified with a detectable label, such as an enzymatic, fluorescent, radioisotopic or affinity label to allow for detection and isolation of the antibody.

Examples of suitable enzymes include horseradish peroxidase, alkaline phosphatase, beta-galactosidase, glucose oxidase or acetylcholinesterase; examples of suitable prosthetic group complexes include streptavidin/biotin and avidin/biotin; examples of suitable fluorescent materials include biotin, umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include a radioactive metal ion, e.g., alpha-emitters such as, for example, ²¹³Bi, or other radioisotopes such as, for example, iodine (¹³¹I, ¹²⁵I, ¹²³I, ¹²¹I), carbon (¹⁴C), sulfur (³⁵S), tritium (³H), indium (^{115m}In, ^{113m}In, ¹¹²In, ¹¹¹In), and technetium (⁹⁹Tc, ^{99m}Tc), thallium (²⁰¹Ti), gallium (68Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ¹¹³Sn, and ¹¹⁷Tin.

In specific embodiments, antibodies of the invention may be labeled with Europium. For example, antibodies of the invention may be labelled with Europium using the DELFIA Eu-labeling kit (catalog #1244-302, Perkin Elmer Life Sciences, Boston, Mass.) following manufacturer's instructions.

In specific embodiments, antibodies of the invention are attached to macrocyclic chelators useful for conjugating radiometal ions, including but not limited to, ¹¹¹In, ¹⁷⁷Lu, ⁹⁰Y, ¹⁶⁶Ho, ¹⁵³Sm, ²¹⁵Bi and ²²⁵Ac to polypeptides. In a preferred embodiment, the radiometal ion associated with the macrocyclic chelators attached to antibodies of the invention is ¹¹¹In. In another preferred embodiment, the radiometal ion associated with the macrocyclic chelator attached to antibodies polypeptides of the invention is ⁹⁰Y. In specific embodiments, the macrocyclic chelator is 1,4,7,10-tetraazacyclododecane-N,N′,N″,N′″-tetraacetic acid (DOTA). In specific embodiments, the macrocyclic chelator is α-(5-isothiocyanato-2-methoxyphenyl)-1,4,7,10-tetraaza-cyclododecane-1,4,7,10-tetraacetic acid. In other specific embodiments, the DOTA is attached to the antibody of the invention via a linker molecule. Examples of linker molecules useful for conjugatinga macrocyclic chelator such as DOTA to a polypeptide are commonly known in the art—see, for example, DeNardo et al., Clin Cancer Res. 4(10):2483-90, 1998; Peterson et al., Bioconjug. Chem. 10(4):553-7, 1999; and Zimmerman et al, Nucl. Med. Biol. 26(8):943-50, 1999 which are hereby incorporated by reference in their entirety. In addition, U.S. Pat. Nos, 5,652,361 and 5,756,065, which disclose chelating agents that may be conjugated to antibodies, and methods for making and using them, are hereby incorporated by reference in their entireties.

In one embodiment, antibodies of the invention are labeled with biotin. In other related embodiments, biotinylated antibodies of the invention may be used, for example, as an imaging agent or as a means of identifying one or more TRAIL receptor coreceptor or ligand molecules.

Also provided by the invention are chemically modified derivatives of antibodies of the invention which may provide additional advantages such as increased solubility, stability and in vivo or in vitro circulating time of the polypeptide, or decreased immunogenicity (see U.S. Pat. No. 4,179,337). The chemical moieties for derivitization may be selected from water soluble polymers such as polyethylene glycol, ethylene glycol/propylene glycol copolymers, carboxymethylcellulose, dextran, polyvinyl alcohol and the like. The antibodies may be modified at random positions within the molecule, or at predetermined positions within the molecule and may include one, two, three or more attached chemical moieties.

The polymer may be of any molecular weight, and may be branched or unbranched. For polyethylene glycol, the preferred molecular weight is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog). For example, the polyethylene glycol may have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 25,000, 30,000, 35,000, 40,000, 50,000, 55,000, 60,000, 65,000, 70,000, 75,000, 80,000, 85,000, 90,000, 95,000, or 100,000 kDa.

As noted above, the polyethylene glycol may have a branched structure. Branched polyethylene glycols are described, for example, in U.S. Pat. No. 5,643,575; Morpurgo et al., Appl. Biochem. Biotechnol. 56:59-72 (1996); Vorobjev et al., Nucleosides Nucleotides 18:2745-2750 (1999); and Caliceti et al., Bioconjug. Chem. 10:638-646 (1999), the disclosures of each of which are incorporated herein by reference.

Characterization of Anti-TR2 Antibodies

Antibodies of the present invention (including molecules comprising, or alternatively consisting of, antibody fragments or variants thereof) may also be described or specified in terms of their binding to TR2 polypeptides or fragments or variants of TR2 polypeptides. In specific embodiments, antibodies of the invention bind TR2 polypeptides, or fragments or variants thereof, with a dissociation constant or K_{D} of less than or equal to 5×10⁻² M, 10⁻² M, 5×10⁻³ M, 10⁻³ M, 5×10⁻⁴ M, 10⁻⁴M, or 10⁻⁵ M. More preferably, antibodies of the invention bind TR2 polypeptides or fragments or variants thereof with a dissociation constant or K_{D} less than or equal to 5×10⁻⁶ M, 10⁻⁶ M, 5×10⁻⁷ M, 10⁻⁷ M, 5×10⁻⁸ M, or 10⁻⁸ M. Even more preferably, antibodies of invention bind TR2 polypeptides or fragments or variants thereof with a dissociation constant or K_{D} less than or equal to 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M. The invention encompasses antibodies that bind TR2 polypeptides with a dissociation constant or K_{D} that is within any one of the ranges that are between each of the individual recited values.

In specific embodiments, antibodies of the invention bind TR2 polypeptides or fragments or variants thereof with an off rate (k_{off}) of less than or equal to 5×10⁻² sec⁻¹, 10⁻² sec⁻¹, 5×10⁻³ sec⁻¹ or 10⁻³ sec⁻¹. More preferably, antibodies of the invention bind TR2 polypeptides or fragments or variants thereof with an off rate (k_{off}) less than or equal to 5×10⁻⁴ sec⁻¹, 10⁻⁴ sec−1, 5×10⁻⁵ sec⁻¹, or 10⁻⁵ sec⁻¹ 5×10⁻⁶ sec⁻¹, 10⁻⁶ sec⁻¹, 5×10⁻⁷ sec⁻¹ or 10⁻⁷ sec⁻¹. The invention encompasses antibodies that bind TR2 polypeptides with an off rate (k_{off}) that is within any one of the ranges that are between each of the individual recited values.

In other embodiments, antibodies of the invention bind TR2 polypeptides or fragments or variants thereof with an on rate (kₒₙ) of greater than or equal to 10³ M⁻¹ sec⁻¹, 5×10³ M⁻¹ sec⁻¹, 10⁴ M⁻¹ sec⁻¹ or 5×10⁴ M⁻¹ sec⁻¹. More preferably, antibodies of the invention bind TR2 polypeptides or fragments or variants thereof with an on rate (kₒₙ) greater than or equal to 10⁵ M⁻¹ sec⁻¹, 5×10⁵ M⁻¹ sec⁻¹, 10⁶ M⁻¹ sec⁻1, or 5×10⁶ M sec⁻¹ or 10⁷ M⁻¹ sec⁻¹. The invention encompasses antibodies that bind TR2 polypeptides with on rate (kₒₙ) that is within any one of the ranges that are between each of the individual recited values.

The antibodies of the invention (including molecules comprising, or alternatively consisting of, antibody fragments or variants thereof) specifically bind to a polypeptide or polypeptide fragment or variant of human TR2 polypeptides (SEQ ID NOS:2, 4, or 6). In another embodiment, the antibodies of the invention specifically bind to a polypeptide or polypeptide fragment or variant of simian TR2 polypeptides. In yet another embodiment, the antibodies of the invention specifically bind to a polypeptide or polypeptide fragment or variant of murine TR2 polypeptides. In one embodiment, the antibodies of the invention bind specifically to human and simian TR2 polypeptides. In another embodiment, the antibodies of the invention bind specifically to human TR2 polypeptides and murine TR2 polypeptides. More preferably, antibodies of the invention, preferentially bind to human TR2 polypeptides compared to murine TR2 polypeptides.

In preferred embodiments, the antibodies of the present invention (including molecules comprising, or alternatively consisting of, antibody fragments or variants thereof), specifically bind to TR2 polypeptides and do not cross-react with any other antigens. In preferred embodiments, the antibodies of the invention specifically bind to TR2 polypeptides (e.g., SEQ ID NOS:2, 4, 6 or fragments or variants thereof) and do not cross-react with one or more additional members of the Tumor Necrosis Factor Tumor Necrosis Factor Receptor Family polypeptides (e.g., BCMA, TACI, CD30, CD27, OX40, 4-1BB, CD40, NGFR, TNFR1, TNFR2, Fas, and NGFR).

In another embodiment, the antibodies of the present invention (including molecules comprising, or alternatively consisting of, antibody fragments or variants thereof), specifically bind to TR2 polypeptides and cross-react with other antigens. In other embodiments, the antibodies of the invention specifically bind to TR2 polypeptides (e.g., SEQ ID NOS:2, 4, 6, or fragments or variants thereof) and cross-react with one or more additional members of the Tumor Necrosis Factor Receptor Family polypeptides (e.g., BCMA, TACI, CD30, CD27, OX40, 4-1BB, CD40, NGFR, TNFR1, TNFR2, Fas, and NGFR).

In a preferred embodiment, antibodies of the invention preferentially bind TR2 (SEQ ID NO:2), or fragments and variants thereof relative to their ability to bind TR2-SV1 or TR2-SV2 (SEQ ID NOS:4 and 6) or fragments or variants thereof.

In another embodiment, antibodies of the invention preferentially bind TR2-SV1 (SEQ ID NO:4), or fragments and variants thereof relative to their ability to bind TR2 or TR2-SV2 (SEQ ID NOS:2 and 6) or fragments or variants thereof. In other embodiments, the antibodies of the invention preferentially bind to TR2 and TR2-SV2 (SEQ ID NOS:2 and 6), or fragments and variants thereof relative to their ability to bind TR2-SV1 (SEQ ID NO:4) or fragments or variants thereof. In other embodiments, the antibodies of the invention preferentially bind to TR2 and TR2-SV1 (SEQ ID NOS:2 and 4), or fragments and variants thereof relative to their ability to bind TR2-SV2 (SEQ ID NO:6) or fragments or variants thereof. In other embodiments, the antibodies of the invention preferentially bind to TR2-SV1 and TR2-SV2 (SEQ ID NOS:4 and 6), or fragments and variants thereof relative to their ability to bind TR2 (SEQ ID NO:2) or fragments or variants thereof. In other embodiments, the antibodies of the invention bind TR2, TR2-SV1, and TR2-SV2 (SEQ ID NOS:2, 4, and 6). In another embodiment, antibodies of the invention preferentially bind TR2-SV2 (SEQ ID NO:6), or fragments and variants thereof relative to their ability to bind TR2 or TR2-SV1 (SEQ ID NOS:2 and 6) or fragments or variants thereof. An antibody's ability to preferentially bind one antigen compared to another antigen may be determined using any method known in the art.

By way of non-limiting example, an antibody may be considered to bind a first antigen preferentially if it binds said first antigen with a dissociation constant (K_{D}) that is less than the antibody's K_{D} for the second antigen. In another non-limiting embodiment, an antibody may be considered to bind a first antigen preferentially if it binds said first antigen with an affinity (i.e., K_{D}) that is at least one order of magnitude less than the antibody's K_{D} for the second antigen. In another non-limiting embodiment, an antibody may be considered to bind a first antigen preferentially if it binds said first antigen with an affinity (i.e., K_{D}) that is at least two orders of magnitude less than the antibody's K_{D} for the second antigen.

In another non-limiting embodiment, an antibody may be considered to bind a first antigen preferentially if it binds said first antigen with an off rate (k_{off}) that is less than the antibody's k_{off} for the second antigen. In another non-limiting embodiment, an antibody may be considered to bind a first antigen preferentially if it binds said first antigen with a k_{off} that is at least one order of magnitude less than the antibody's k_{off} for the second antigen. In another non-limiting embodiment, an antibody may be considered to bind a first antigen preferentially if it binds said first antigen with a k_{off} that is at least two orders of magnitude less than the antibody's k_{off} for the second antigen.

The invention also encompasses antibodies (including molecules comprising, or alternatively consisting of, antibody fragments or variants thereof) that have one or more of the same biological characteristics as one or more of the antibodies described herein. By “biological characteristics” is meant, the in vitro or in vivo activities or properties of the antibodies, such as, for example, the ability to bind to TR2 polypeptides (e.g., TR2 proteins expressed on the surface of a cell), the ability to inhibit TR2 mediated biological activity (e.g., to inhibit proliferation and/or differentiation of TR2 expressing cells; See Example 3); the ability to inhibit or substantially block binding of a TR2 ligand (e.g. LIGHT (also referred to as LIGHT; International Publication Nos. WO 97/34911 and WO 99/42584; Zhai et al., J. Clin. Invest. 102:1142-1151 (1998); SEQ ID NO:45), and LTα (also referred to as lymphotoxin-α and TNF-β; Granger et al., Nature 218:1253-4 (1968); SEQ ID NO:46), which are hereby incorporated by reference in their entirety, or a fragment, variant or fusion protein thereof, to TR2; the ability to inhibit or substantially block TR2-mediated entry of Herpes simplex virus (HSV) into cells; or the ability to downregulate TR2 expression on the surface of cells. Other biological activities that antibodies against TR2 polypeptides may have, include, but are not limited to, the ability to stimulate TR2 mediated biological activity (e.g., to stimulate proliferation and/or differentiation of TR2 expressing cells (e.g., T-cells)) or the ability to upregulate TR2 expression on the surface of cells. Optionally, the antibodies of the invention will bind to the same epitope as at least one of the antibodies specifically referred to herein. Such epitope binding can be routinely determined using assays known in the art.

The present invention also provides for antibodies (including molecules comprising, or alternatively consisting of, antibody fragments or variants thereof), that inhibit one or more TR2 polypeptide mediated biological activities. In one embodiment, an antibody that inhibits one or more TR2 polypeptide mediated biological activities comprises, or alternatively consists of a VH and/or a VL domain of at least one of the scFvs referred to in Table 1, or fragment or variant thereof. In a specific embodiment, an antibody that inhibits one or more TR2 polypeptide mediated biological activities comprises, or alternatively consists of a VH and a VL domain of any one of the scFvs referred to in Table 1, or fragment or variant thereof. Nucleic acid molecules encoding these antibodies are also encompassed by the invention.

The present invention also provides for antibodies (including molecules comprising, or alternatively consisting of, antibody fragments or variants thereof), that inhibit proliferation of TR2 expressing cells, for example T-cells (See Example 3). In one embodiment, an antibody that inhibits proliferation of TR2 expressing cells comprises, or alternatively consists of a VH and/or a VL domain of at least one of the scFvs referred to in Table 1, or fragment or variant thereof. In a specific embodiment, an antibody that inhibits proliferation of TR2 expressing cells comprises, or alternatively consists of a VH and a VL domain of any one of the scFvs referred to in Table 1, or fragment or variant thereof. Nucleic acid molecules encoding these antibodies are also encompassed by the invention.

The present invention also provides for antibodies (including molecules comprising, or alternatively consisting of, antibody fragments or variants thereof), that inhibit differentiation of TR2 expressing cells (e.g., T-cells). In one embodiment, an antibody that inhibits differentiation of TR2 expressing cells comprises, or alternatively consists of a VH and/or a VL domain of at least one of the scFvs referred to in Table 1, or fragment or variant thereof. In a specific embodiment, an antibody that inhibits differentiation of TR2 expressing cells comprises, or alternatively consists of a VH and a VL domain of any one of the scFvs referred to in Table 1, or fragment or variant thereof. Nucleic acid molecules encoding these antibodies are also encompassed by the invention.

The present invention also provides for antibodies (including molecules comprising, or alternatively consisting of, antibody fragments or variants thereof), that inhibit HSV entry into TR2 expressing cells. In one embodiment, an antibody that inhibits HSV entry into TR2 expressing cells comprises, or alternatively consists of a VH and/or a VL domain of at least one of the scFvs referred to in Table 1, or fragment or variant thereof. In a specific embodiment, an antibody that inhibits HSV entry into TR2 expressing cells comprises, or alternatively consists of a VH and a VL domain of any one of the scFvs referred to in Table 1, or fragment or variant thereof. Nucleic acid molecules encoding these antibodies are also encompassed by the invention.

The present invention also provides for antibodies (including molecules comprising, or alternatively consisting of, antibody fragments or variants thereof), that block or inhibit the binding of TR2 ligand to a TR2 polypeptide. In one embodiment, an antibody that blocks or inhibits the binding of TR2 ligand to a TR2 polypeptide comprises, or alternatively consists of a VH and/or a VL domain of at least one of the scFvs referred to in Table 1, or fragment or variant thereof. In a specific embodiment, an antibody that blocks or inhibits the binding of TR2 ligand to a TR2 polypeptide comprises, or alternatively consists of a VH and a VL domain of any one of the scFvs referred to in Table 1, or fragment or variant thereof. In one specific embodiment the TR2 ligand is LIGHT (SEQ ID NO:45). In another specific embodiment the TR2 ligand is LTα (SEQ ID NO:46). Nucleic acid molecules encoding these antibodies are also encompassed by the invention.

The present invention also provides for antibodies (including molecules comprising, or alternatively consisting of, antibody fragments or variants thereof), that stimulate TR2 expressing cells (e.g., T-cells). In one embodiment, an antibody that stimulates TR2 expressing cells comprises, or alternatively consists of a VH and/or a VL domain of at least one of the scFvs referred to in Table 1, or fragment or variant thereof. In a specific embodiment, an antibody that stimulates TR2 expressing cells comprises, or alternatively consists of a VH and a VL domain of any one of the scFvs referred to in Table 1, or fragment or variant thereof. Nucleic acid molecules encoding these antibodies are also encompassed by the invention.

Antibodies of the present invention (including antibody fragments or variants thereof) may be characterized in a variety of ways. In particular, antibodies and related molecules of the invention may be assayed for the ability to specifically bind to TR2 polypeptides or a fragment or variant of TR2 polypeptides using techniques described herein or routinely modifying techniques known in the art. Assays for the ability of the antibodies of the invention to specifically bind TR2 polypeptides or a fragment of TR2 may be performed in solution (e.g., Houghten, Bio/Techniques 13:412-421(1992)), on beads (e.g., Lam, Nature 354:82-84 (1991)), on chips (e.g., Fodor, Nature 364:555-556 (1993)), on bacteria (e.g., U.S. Pat. No. 5,223,409), on spores (e.g., U.S. Pat. Nos. 5,571,698; 5,403,484; and 5,223,409), on plasmids (e.g., Cull et al., Proc. Natl. Acad. Sci. USA 89:1865-1869 (1992)) or on phage (e.g., Scott and Smith, Science 249:386-390 (1990); Devlin, Science 249:404-406 (1990); Cwirla et al., Proc. Natl. Acad. Sci. USA 87:7178-7182 (1990); and Felici, J. Mol. Biol. 222:301-310 (1991)) (each of these references is incorporated herein in its entirety by reference). Antibodies that have been identified to specifically bind to TR2 polypeptides or a fragment or variant of TR2 ,can then be assayed for their specificity and affinity for TR2 polypeptides or a fragment or variant of a TR2 polypeptide using or routinely modifying techniques described herein or otherwise known in the art.

The antibodies of the invention may be assayed for specific binding to TR2 polypeptides and cross-reactivity with other antigens by any method known in the art. Immunoassays which can be used to analyze specific binding and cross-reactivity include, but are not limited to, competitive and non-competitive assay systems using techniques such as BlAcore analysis, FACS (fluorescence activated cell sorter) analysis, immunofluorescence, immunocytochemistry, radioimmunoassays, ELISA (enzyme linked immunosorbent assay), “sandwich” immunoassays, immunoprecipitation assays, western blots, precipitin reactions, gel diffusion precipitin reactions, immunodiffusion assays, agglutination assays, complement-fixation assays, immunoradiometric assays, fluorescent immunoassays, and protein A immunoassays, to name but a few. Such assays are routine and well known in the art (see, e.g., Ausubel et al., eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

ELISAs comprise preparing antigen, coating the well of a 96-well microtiter plate with the antigen, washing away antigen that did not bind the wells, adding the antibody of interest conjugated to a detectable compound such as an enzymatic substrate (e.g., horseradish peroxidase or alkaline phosphatase) to the wells and incubating for a period of time, washing away unbound antibodies or non-specifically bound antibodies, and detecting the presence of the antibodies specifically bound to the antigen coating the well. In ELISAs, the antibody of interest does not have to be conjugated to a detectable compound; instead, a second antibody (which recognizes the antibody of interest) conjugated to a detectable compound may be added to the well. Alternatively, the antigen need not be directly coated to the well; instead the ELISA plates may be coated with an anti-Ig Fc antibody, and the antigen in the form or a TR2-Fc fusion protein, may be bound to the anti-Ig Fc coated to the plate. This may be desirable so as to maintain the antigen protein (e.g., the TR2 polypeptides) in a more native conformation than it may have when it is directly coated to a plate. In another alternative, instead of coating the well with the antigen, the antibody may be coated to the well. In this case, the detectable molecule could be the antigen conjugated to a detectable compound such as an enzymatic substrate (e.g., horseradish peroxidase or alkaline phosphatase). One of skill in the art would be knowledgeable as to the parameters that can be modified to increase the signal detected as well as other variations of ELISAs known in the art. For further discussion regarding ELISAs see, e.g., Ausubel et al., eds, 1994, Current Protocols in Molecular Biology, Vol. 1, John Wiley & Sons, Inc., New York at 11.2.1.

The binding affinity of an antibody (including an scFv or other molecule comprising, or alternatively consisting of, antibody fragments or variants thereof) to an antigen and the off-rate of an antibody-antigen interaction can be determined by competitive binding assays. One example of a competitive binding assay is a radioimmunoassay comprising the incubation of labeled antigen (e.g., antigen labeled with ³H or ¹²⁵I), or fragment or variant thereof with the antibody of interest in the presence of increasing amounts of unlabeled antigen, and the detection of the antibody bound to the labeled antigen. The affinity of the antibody of the present invention for TR2 and the binding off-rates can be determined from the data by Scatchard plot analysis. Competition with a second antibody can also be determined using radioimmunoassays. In this case, TR2 polypeptide is incubated with an antibody of the present invention conjugated to a labeled compound (e.g., compound labeled with ³H or ¹²⁵I) in the presence of increasing amounts of an unlabeled second anti-TR2 antibody. This kind of competitive assay between two antibodies, may also be used to determine if two antibodies bind the same, closely associated (e.g., overlapping) or different epitopes.

In a preferred embodiment, BlAcore kinetic analysis is used to determine the binding on and off rates of antibodies (including antibody fragments or variants thereof) to TR2, or fragments of TR2. BlAcore kinetic analysis comprises analyzing the binding and dissociation of antibodies from chips with immobilized TR2 proteins on their surface.

Immunoprecipitation protocols generally comprise lysin a population of cells in a lysis buffer such as RIPA buffer. (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1 to 4 hours) at 40 degrees C., adding protein A and/or protein G sepharose beads to the cell lysate, incubating for about an hour or more at 40 degrees C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al., eds, 1994, Current Protocols in Molecular Biology, Vol. 1, John Wiley & Sons, Inc., New York at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., ³²P or ¹²⁵I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al., eds, 1994, Current Protocols in Molecular Biology, Vol. 1, John Wiley & Sons, Inc., New York at 10.8.1.

Antibody Conjugates

The present invention encompasses antibodies (including antibody fragments or variants thereof), recombinantly fused or chemically conjugated (including both covalent and non-covalent conjugations) to a heterologous polypeptide (or portion thereof, preferably at least 10, at least 20, at least 30, at least 40, at least 50, at least 60, at least 70, at least 80, at least 90 or at least 100 amino acids of the polypeptide) to generate fusion proteins. The fusion does not necessarily need to be direct, but may occur through linker sequences. For example, antibodies of the invention may be used to target heterologous polypeptides to particular cell types (e.g., cancer cells), either in vitro or in vivo, by fusing or conjugating the heterologous polypeptides to antibodies of the invention that are specific for particular cell surface antigens or which bind antigens that bind particular cell surface receptors. Antibodies of the invention may also be fused to albumin (including but not limited to recombinant human serum albumin (see, e.g., U.S. Pat. No. 5,876,969, issued Mar. 2, 1999, EP Patent 0 413 622, and U.S. Pat. No. 5,766,883, issued Jun. 16, 1998, herein incorporated by reference in their entirety)), resulting in chimeric polypeptides. In a preferred embodiment, polypeptides and/or antibodies of the present invention (including fragments or variants thereof) are fused with the mature form of human serum albumin (i.e., amino acids 1-585 of human serum albumin as shown in FIGS. 1 and 2 of EP Patent 0 322 094) which is herein incorporated by reference in its entirety. In another preferred embodiment, polypeptides and/or antibodies of the present invention (including fragments or variants thereof) are fused with polypeptide fragments comprising, or alternatively consisting of, amino acid residues 1-z of human serum albumin, where z is an integer from 369 to 419, as described in U.S. Pat. 5,766,883 herein incorporated by reference in its entirety. Polypeptides and/or antibodies of the present invention (including fragments or variants thereof) may be fused to either the N- or C-terminal end of the heterologous protein (e.g., immunoglobulin Fc polypeptide or human serum albumin polypeptide). Polynucleotides encoding fusion proteins of the invention are also encompassed by the invention. Such fusion proteins may, for example, facilitate purification and may increase half-life in vivo. Antibodies fused or conjugated to heterologous polypeptides may also be used in in vitro immunoassays and purification methods using methods known in the art. See e.g., Harbor et al., supra, and PCT publication WO 93/2 1232; EP 439,095; Naramura et al., Immunol. Lett. 39:91-99 (1994); U.S. Pat. 5,474,981; Gillies et al., PNAS 89:1428-1432 (1992); Fell et al., J. Immunol. 146:2446-2452 (1991), which are incorporated by reference in their entireties.

The present invention further includes compositions comprising, or alternatively consisting of, heterologous polypeptides fused or conjugated to antibody fragments. For example, the heterologous polypeptides may be fused or conjugated to a Fab fragment, Fd fragment, Fv fragment, F(ab)₂ fragment, or a portion thereof. Methods for fusing or conjugating polypeptides to antibody portions are known in the art. See, e.g., U.S. Pat. Nos. 5,356,603; 5,622,929; 5,359,046; 5,349,053; 5,447,851; 5,112,946; EP 307,434; EP 367,166; PCT publications WO 96/04388; WO 9 1/06570; Ashkenazi et al., Proc. Natl. Acad. Sci. USA 88: 10535-10539 (1991); Zheng et al., J. Immunol. 154:5590-5600 (1995); and Vil et al., Proc. Natl. Acad. Sci. USA 89:11357-11341 (1992) (said references incorporated by reference in their entireties).

Additional fusion proteins of the invention may be generated through the techniques of gene-shuffling, motif-shuffling, exon-shuffling, and/or codon-shuffling (collectively referred to as “DNA shuffling”). DNA shuffling may be employed to modulate the activities of antibodies (including molecules comprising, or alternatively consisting of, antibody fragments or variants thereof), such methods can be used to generate antibodies with altered activity (e.g., antibodies with higher affinities and lower dissociation rates). See, generally, U.S. Pat. Nos. 5,605,793; 5,811,238; 5,830,721; 5,834,252; and 5,837,458, and Patten et al., Curr. Opinion Biotechnol. 8:724-35 (1997); Harayama, Trends Biotechnol. 16(2):76-82 (1998); Hansson, et al., J. Mol. Biol. 287:265-76 (1999); and Lorenzo and Blasco, Biotechniques 24(2):308-13 (1998) (each of these patents and publications are hereby incorporated by reference in its entirety). In one embodiment, polynucleotides encoding antibodies of the invention may be altered by being subjected to random mutagenesis by error-prone PCR, random nucleotide insertion or other methods prior to recombination. In another embodiment, one or more portions of a polynucleotide encoding an antibody which portions specifically bind to TR2 may be recombined with one or more components, motifs, sections, parts, domains, fragments, etc. of one or more heterologous molecules.

Moreover, the antibodies of the present invention (including antibody fragments or variants thereof), can be fused to marker sequences, such as a polypeptides to facilitate purification. In preferred embodiments, the marker amino acid sequence is a hexa-histidine polypeptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. Other peptide tags useful for purification include, but are not limited to, the hemagglutinin “HA” tag, which corresponds to an epitope derived from the influenza hemagglutinin protein (Wilson et al., Cell 37:767 (1984)) and the FLAG® tag (Stratagene, La Jolla, Calif.).

The present invention further encompasses antibodies (including antibody fragments or variants thereof), conjugated to a diagnostic or therapeutic agent. The antibodies can be used diagnostically to, for example, monitor or prognose the development or progression of a tumor as part of a clinical testing procedure to, e.g., determine the efficacy of a given treatment regimen. Detection can be facilitated by coupling the antibody to a detectable substance. Examples of detectable substances include, but are not limited to, various enzymes, prosthetic groups, fluorescent materials, luminescent materials, bioluminescent materials, radioactive materials, positron emitting metals using various positron emission tomographies, and nonradioactive paramagnetic metal ions. The detectable substance may be coupled or conjugated either directly to the antibody or indirectly, through an intermediate (such as, for example, a linker known in the art) using techniques known in the art. See, for example, U.S. Pat. No. 4,741,900 for metal ions which can be conjugated to antibodies for use as diagnostics according to the present invention. Examples of suitable enzymes include, but are not limited to, horseradish peroxidase, alkaline phosphatase, beta-galactosidase, or acetylcholinesterase; examples of suitable prosthetic group complexes include, but are not limited to, streptavidin/biotin and avidin/biotin; examples of suitable fluorescent materials include, but are not limited to, umbelliferone, fluorescein, fluorescein isothiocyanate, rhodamine, dichlorotriazinylamine fluorescein, dansyl chloride or phycoerythrin; an example of a luminescent material includes, but is not limited to, luminol; examples of bioluminescent materials include, but are not limited to, luciferase, luciferin, and aequorin; and examples of suitable radioactive material include, but are not limited to, iodine (¹²¹I, ¹²³I, ¹²⁵I, ¹³¹I), carbon (¹⁴C), sulfur (³⁵S), tritium (³H), indium (¹¹¹In, ¹¹²In, ^{113m}In, ^{115m}In), technetium (⁹⁹Tc,^{99m}Tc), thallium (²⁰¹Ti), gallium (⁸⁸Ga, ⁶⁷Ga), palladium (¹⁰³Pd), molybdenum (⁹⁹Mo), xenon (¹³⁵Xe), fluorine (¹⁸F), ¹⁵³Sm, ¹⁷⁷Lu, ¹⁵⁹Gd, ¹⁴⁹Pm, ¹⁴⁰La, 175Yb, ¹⁶⁶Ho, ⁹⁰Y, ⁴⁷Sc, ¹⁸⁶Re, ¹⁸⁸Re, ¹⁴²Pr, ¹⁰⁵Rh, and ⁹⁷Ru.

Further, an antibody of the invention (including an scFv or other molecule comprising, or alternatively consisting of, antibody fragments or variants thereof), may be coupled or conjugated to a therapeutic moiety such as a cytotoxin, e.g., a cytostatic or cytocidal agent, a therapeutic agent or a radioactive metal ion, e.g., alpha-emitters such as, for example, ²¹³Bi, or other radioisotopes such as, for example, ¹⁰³Pd, ¹³⁵Xe, ¹³¹I, ⁶⁸Ge, ⁵⁷Co, ⁶⁵ Zn, ⁸⁵Sr, ³²p, ³⁵S, ⁹⁰Y, ¹⁵³Sm, ¹⁵³Gd, 169Yb, ⁵¹Cr, ⁵⁴Mn, ⁷⁵Se, ¹¹³Sn, ⁹⁰Y, ¹¹⁷Tin, ¹⁹⁶Re, ¹⁸⁸Re and ¹⁶⁶Ho. In specific embodiments, an antibody or fragment thereof is attached to macrocyclic chelators that chelate radiometal ions, including but not limited to, ¹⁷⁷Lu, ⁹⁰Y, ¹⁶⁶Ho, and ¹⁵³Sm, to polypeptides. In specific embodiments, the macrocyclic chelator is 1,4,7,10-tetraazacyclododecane-N,N′,N″,N′″-tetraacetic acid (DOTA). In other specific embodiments, the DOTA is attached to the an antibody of the invention or fragment thereof via a linker molecule. Examples of linker molecules useful for conjugating DOTA to a polypeptide are commonly known in the art—see, for example, DeNardo et al., Clin Cancer Res. 4(10):2483-90, 1998; Peterson et al., Bioconjug. Chem. 10(4):553-7, 1999; and Zimmerman et al., Nucl. Med. Biol. 26(8):943-50, 1999 which are hereby incorporated by reference in their entirety.

A cytotoxin or cytotoxic agent includes any agent that is detrimental to cells. Examples include, but are not limited to, paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, thymidine kinase, endonuclease, RNAse, and puromycin and frragments, variants or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Techniques known in the art may be applied to label antibodies of the invention. Such techniques include, but are not limited to, the use of bifunctional conjugating agents (see e.g., U.S. Pat. Nos. 5,756,065; 5,714,711; 5,696,239; 5,652,371; 5,505,931; 5,489,425; 5,435,990; 5,428,139; 5,342,604; 5,274,119; 4,994,560; and 5,808,003; the contents of each of which are hereby incorporated by reference in its entirety) and direct coupling reactions (e.g., Bolton-Hunter and Chloramine-T reaction).

The antibodies of the invention which are conjugates can be used for modifying a given biological response, the therapeutic agent or drug moiety is not to be construed as limited to classical chemical therapeutic agents. For example, the drug moiety may be a protein or polypeptide possessing a desired biological activity. Such proteins may include, but are not limited to, for example, a toxin such as abrin, ricin A, alpha toxin, pseudomonas exotoxin, or diphtheria toxin, saporin, momordin, gelonin, pokeweed antiviral protein, alpha-sarcin and cholera toxin; a protein such as tumor necrosis factor, alpha-interferon, beta-interferon, nerve growth factor, platelet derived growth factor, tissue plasminogen activator, an apoptotic agent, e.g., TNF-alpha, TNF-beta, AIM I (see, International Publication No. WO 97/35899), AIM II (see, International Publication No. WO 97/34911), Fas Ligand (Takahashi et al., Int Immunol., 6:1567-1574 (1994)), VEGI (see, International Publication No. WO 99/23105), a thrombotic agent or an anti-angiogenic agent, e.g., angiostatin or endostatin; or, biological response modifiers such as, for example, lymphokines, interleukin-1 (IL-1), interleukin-2 (IL-2), interleukin-6 (IL-6), granulocyte macrophage colony stimulating factor (GM-CSF), granulocyte colony stimulating factor (G-CSF), or other growth factors.

Antibodies of the invention (including antibody fragments or variants thereof), may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

Techniques for conjugating a therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev. 62:119-58 (1982).

Alternatively, an antibody of the invention can be conjugated to a second antibody to form an antibody heteroconjugate as described by Segal in U.S. Pat. No. 4,676,980, which is incorporated herein by reference in its entirety.

An antibody of the invention (including an other molecules comprising, or alternatively consisting of, an antibody fragment or variant thereof), with or without a therapeutic moiety conjugated to it, administered alone or in combination with cytotoxic factor(s) and/or cytokine(s) can be used as a therapeutic.

Uses of Antibodies of the Invention

Antibodies of the present invention may be used, for example, but not limited to, to purify, detect, and target the polypeptides of the present invention, including both in vitro and in vivo diagnostic and therapeutic methods. For example, the antibodies have use in immunoassays for qualitatively and quantitatively measuring levels of TR2 polypeptides in biological samples. See, e.g., Harlow et al., Antibodies: A Laboratory Manual, (Cold Spring Harbor Laboratory Press, 2nd ed. 1988) (incorporated by reference herein in its entirety).

Immunophenotyping

The antibodies of the invention may be utilized for immunophenotyping of cell lines and biological samples. The translation product of the gene of the present invention may be useful as a cell specific marker, or more specifically as a cellular marker that is differentially expressed at various stages of differentiation and/or maturation of particular cell types, such as T cells (e.g., activated T cells). In other embodiments, the antibodies of the invention may be useful as tumor and/or cancer cell markers. Monoclonal antibodies directed against a specific epitope, or combination of epitopes, will allow for the screening of cellular populations expressing the marker. Various techniques can be utilized using monoclonal antibodies to screen for cellular populations expressing the marker(s), and include magnetic separation using antibody-coated magnetic beads, “panning” with antibody attached to a solid matrix (i.e., plate), and flow cytometry (See, e.g., U.S. Pat. 5,985,660; and Morrison et al., Cell, 96:737-49 (1999)).

These techniques allow for the screening of particular populations of cells, such as might be found with hematological malignancies (i.e. minimal residual disease (MRD) in acute leukemic patients) and “non-self” cells in transplantations to prevent Graft-versus-Host Disease (GVHD). Alternatively, these techniques allow for the screening of hematopoietic stem and progenitor cells capable of undergoing proliferation and/or differentiation, as might be found in human umbilical cord blood.

Epitope Mapping

The present invention provides antibodies (including antibody fragments or variants thereof), that can be used to identify epitopes of a TR2 polypeptide. In particular, the antibodies of the present invention can be used to identify epitopes of a human TR2 polypeptide (e.g., SEQ ID NOS:2, 4, and/or 6) or a TR2 polypeptide expressed on human cells; a murine TR2 or a TR2 polypeptide expressed on murine cells; a rat TR2 polypeptide or a TR2 polypeptide expressed on rat cells; or a monkey TR2 polypeptide or a TR2 polypeptide expressed on monkey cells, using techniques described herein or otherwise known in the art. Fragments which function as epitopes may be produced by any conventional means. (See, e.g., Houghten, Proc. Natl. Acad. Sci. USA 82:5131-5135 (1985), further described in U.S. Pat. No. 4,711,211.) Identified epitopes of antibodies of the present invention may, for example, be used as vaccine candidates, i.e., to immunize an individual to elicit antibodies against the naturally occuring forms of TR2 polypeptides.

Diagnostic Uses of Antibodies

Labeled antibodies of the invention (including molecules comprising, or alternatively consisting of, antibody fragments or variants thereof) which specifically bind to a TR2 polypeptide can be used for diagnostic purposes to detect, diagnose, prognose, or monitor diseases and/or disorders. In specific embodiments, labeled antibodies of the invention (including molecules comprising, or alternatively consisting of, antibody fragments or variants thereof) which specifically bind to a TR2 polypeptide can be used for diagnostic purposes to detect, diagnose, prognose, or monitor diseases and/or disorders associated with the aberrant expression and/or activity of a TR2 polypeptide.

The invention provides for the detection of expression of a TR2 polypeptide comprising: (a) assaying the expression of a TR2 polypeptide in a biological sample from an individual using one or more antibodies of the invention that specifically binds to a TR2 polypeptide; and (b) comparing the level of a TR2 polypeptide with a standard level of a TR2 polypeptide, (e.g., the level in normal biological samples).

The invention provides for the detection of aberrant expression of a TR2 polypeptide comprising: (a) assaying the expression of a TR2 polypeptide in a biological sample from an individual using one or more antibodies of the invention that specifically binds to a TR2 polypeptide; and (b) comparing the level of a TR2 polypeptide with a standard level of a TR2 polypeptide, e.g., in normal biological samples, whereby an increase or decrease in the assayed level of a TR2 polypeptide compared to the standard level of a TR2 polypeptide is indicative of aberrant expression.

By “biological sample” is intended any fluids and/or cells obtained from an individual, body fluid, body tissue, body cell, cell line, tissue culture, or other source which may contain a TR2 polypeptide protein or mRNA. Body fluids include, but are not limited to, sera, plasma, urine, synovial fluid, spinal fluid, saliva, and mucous. Tissues samples may be taken from virtually any tissue in the body. Tissue samples may also be obtained from autopsy material. Methods for obtaining tissue biopsies and body fluids from mammals are well known in the art. Where the biological sample is to include mRNA, a tissue biopsy is the preferred source.

One aspect of the invention is the detection and diagnosis of a disease or disorder associated with aberrant expression of a TR2 polypeptide in an animal, preferably a mammal and most preferably a human. In one embodiment, diagnosis comprises: a) administering (for example, parenterally, subcutaneously, or intraperitoneally) to a subject an effective amount of a labeled antibody of the invention (including molecules comprising, or alternatively consisting of, antibody fragments or variants thereof) that specifically binds to a TR2 polypeptide; b) waiting for a time interval following the administering for permitting the labeled antibody to preferentially concentrate at sites in the subject where TR2 polypeptide is expressed (and for unbound labeled molecule to be cleared to background level); c) determining background level; and d) detecting the labeled antibody in the subject, such that detection of labeled antibody or fragment thereof above the background level and above or below the level observed in a person without the disease or disorder indicates that the subject has a particular disease or disorder associated with aberrant expression of a TR2 polypeptide or a TR2 ligand. Background level can be determined by various methods including, comparing the amount of labeled molecule detected to a standard value previously determined for a particular system.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ⁹⁹Tc. The labeled antibody will then preferentially accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).

Depending on several variables, including the type of label used and the mode of administration, the time interval following the administration for permitting the labeled molecule to preferentially concentrate at sites in the subject and for unbound labeled molecule to be cleared to background level is 6 to 48 hours or 6 to 24 hours or 6 to 12 hours. In another embodiment the time interval following administration is 5 to 20 days or 5 to 10 days.

In one embodiment, monitoring of the disease or disorder is carried out by repeating the method for diagnosing the disease or disorder, for example, one month after initial diagnosis, six months after initial diagnosis, one year after initial diagnosis, etc.

Presence of the labeled molecule can be detected in the patient using methods known in the art for in vivo scanning. These methods depend upon the type of label used. Skilled artisans will be able to determine the appropriate method for detecting a particular label. Methods and devices that may be used in the diagnostic methods of the invention include, but are not limited to, computed tomography (CT), whole body scan such as position emission tomography (PET), magnetic resonance imaging (MRI), and sonography.

In a specific embodiment, the molecule is labeled with a radioisotope and is detected in the patient using a radiation responsive surgical instrument (Thurston et al., U.S. Pat. No. 5,441,050). In another embodiment, the molecule is labeled with a fluorescent compound and is detected in the patient using a fluorescence responsive scanning instrument. In another embodiment, the molecule is labeled with a positron emitting metal and is detected in the patient using positron emission-tomography. In yet another embodiment, the molecule is labeled with a paramagnetic label and is detected in a patient using magnetic resonance imaging (MRI).

Therapeutic Uses of Antibodies

One or more antibodies of the present invention (including molecules comprising, or alternatively consisting of, antibody fragments or variants thereof) that specifically bind to TR2 may be used locally or systemically in the body as a therapeutic. In one embodiment, the present invention also encompasses antibody-based therapies which involve administering antibodies of the invention (including molecules comprising, or alternatively consisting of, antibody fragments or variants thereof) to an animal, preferably a mammal, and most preferably a human, for preventing or treating one or more of the disclosed diseases, disorders, or conditions. Therapeutic compounds of the invention include, but are not limited to, antibodies of the invention and nucleic acids encoding antibodies (and anti-idiotypic antibodies) of the invention as described herein. In one embodiment, the antibodies of the invention can be used to treat, ameliorate or prevent diseases, disorders or conditions, including, but not limited to, any one or more of the diseases, disorders, or conditions described herein. The treatment and/or prevention of diseases, disorders, or conditions includes, but is not limited to, alleviating symptoms associated with those diseases, disorders or conditions. Antibodies of the invention may be provided in pharmaceutically acceptable compositions as known in the art or as described herein. In certain embodiments, properties of the antibodies of the present invention make the antibodies better therapeutic agents than previously described TR2 binding antibodies.

Therapeutic Uses of Antibodies for Treating Autoimmune Disorders and Immunodeficiencies

In highly preferred embodiments, antibodies of the invention, e.g., antibodies of the invention that bind a TR2 polypeptide and inhibit differentiation of TR2 expressing cells, are used to treat, prevent or ameliorate autoimmune diseases, disorders, or conditions associated with such diseases or disorders. In specific embodiments, antibodies of the invention are used to inhibit the progression of an autoimmune response and other related disorders.

Autoimmune disorders and related disorders, include, but are not limited to, autoimmune hemolytic anemia, autoimmune neonatal thrombocytopenia, idiopathic thrombocytopenia purpura, autoimmunocytopenia, hemolytic anemia, antiphospholipid syndrome, dermatitis, allergic encephalomyelitis, myocarditis, relapsing polychondritis, ulcerative colitis, dense deposit disease, rheumatic heart disease, glomerulonephritis, IgA nephropathy, pemphigus vulgaris, discoid lupus, Multiple Sclerosis, Neuritis, Uveitis Ophthalmia, Polyendocrinopathies, Purpura (e.g., Henloch-Scoenlein purpura), Reiter's Disease, Stiff-Man Syndrome, Autoimmune Pulmonary Inflammation, Guillain-Barre Syndrome, insulin dependent diabetes mellitis, and autoimmune inflammatory eye, autoimmune thyroiditis, hypothyroidism (i.e., Hashimoto's thyroiditis), systemic lupus erhythematosus, Goodpasture's syndrome, Pemphigus, Receptor autoimmunities such as, for example, (a) Graves' Disease , (b) Myasthenia Gravis, and (c) insulin resistance, autoimmune hemolytic anemia, autoimmune thrombocytopenic purpura, rheumatoid arthritis, schleroderma with anti-collagen antibodies, mixed connective tissue disease, polymyositis/dermatomyositis, pernicious anemia, idiopathic Addison's disease, infertility, glomerulonephritis such as primary glomerulonephritis and IgA nephropathy, bullous pemphigoid, Sjogren's syndrome, diabetes millitus, and adrenergic drug resistance (including adrenergic drug resistance with asthma or cystic fibrosis), chronic active hepatitis, primary biliary cirrhosis, other endocrine gland failure, vitiligo, vasculitis, post-MI, cardiotomy syndrome, urticaria, atopic dermatitis, asthma, inflammatory myopathies, graft v. host diseases (GVHD) and other inflammatory, granulamatous, degenerative, and atrophic disorders).

In a specific embodiment, antibodies of the invention are be used to treat, inhibit, prognose, diagnose or prevent rheumatoid arthritis.

In another specific embodiment, antibodies of the invention are used to treat, inhibit, prognose, diagnose or prevent systemic lupus erythematosus.

In another specific embodiment, antibodies of the invention are used to treat, inhibit, prognose, diagnose or prevent graft v. host diseases (GVHD).

Antibodies of the invention may also be used to prevent, diagnose and/or treat autoimmune diseases in which the activity of T cells, particularly T helper 1, Tctl1, and/or CD8+T cells play a role in the pathology of the disease. In specific embodiments, antibodies of the invention are used to treat, inhibit, prognose, diagnose and/or prevent multiple sclerosis. In specific embodiments, antibodies of the invention are used to treat, inhibit, prognose, diagnose and/or prevent myasthenia gravis. In specific embodiments, antibodies of the invention are used to treat, inhibit, prognose, diagnose and/or prevent alzheimer's disease. In specific embodiments, antibodies of the invention are used to treat, inhibit, prognose, diagnose and/or prevent autoimmune diabetes.

Additionally, the antibodies of the invention can be used to treat, inhibit or prevent diseases, disorders or conditions associated with immunodeficiencies including, but not limited to, severe combined immunodeficiency (SCID)-X linked, SCID-autosomal, adenosine deaminase deficiency (ADA deficiency), X-linked agammaglobulinemia (XLA), Bruton's disease, congenital agammaglobulinemia, X-linked infantile agammaglobulinemia, acquired agammaglobulinemia, adult onset agammaglobulinemia, late-onset agammaglobulinemia, dysgammaglobulinemia, hypogammaglobulinemia, transient hypogammaglobulinemia of infancy, unspecified hypogammaglobulinemia, agammaglobulinemia, common variable immunodeficiency (CVID) (acquired), Wiskott-Aldrich Syndrome (WAS), X-linked immunodeficiency with hyper IgM, non X-linked immunodeficiency with hyper IgM, selective IgA deficiency, IgG subclass deficiency (with or without IgA deficiency), antibody deficiency with normal or elevated Igs, immunodeficiency with thymoma, Ig heavy chain deletions, kappa chain deficiency, B cell lymphoproliferative disorder (BLPD), selective IgM immunodeficiency, recessive agammaglobulinemia (Swiss type), reticular dysgenesis, neonatal neutropenia, autoimmune neutropenia, severe congenital leukopenia, thymic alymphoplasia-aplasia or dysplasia with immunodeficiency, ataxia-telangiectasia, short limbed dwarfism, X-linked lymphoproliferative syndrome (XLP), Nezelof syndrome-combined immunodeficiency with Igs, purine nucleoside phosphorylase deficiency (PNP), MHC Class II deficiency (Bare Lymphocyte Syndrome) and severe combined immunodeficiency.

Antibodies of the invention may also be used to prevent graft rejection and inflammation and for the treatment of arthritis.

Therapeutic Uses of Antibodies for Treating Viral Infection

TR2 has been shown to mediate HSV-1 entry into cells. Montgomery, R. et al., Eur. Cytokine Newt. 7:159 (1996). Further, antibodies specific for the extracellular domain of TR2 have been shown to block HSV-1 entry into cells. Therefore, the present invention includes antibodies capable of preventing TR2 mediated viral entry into TR2-expressing cells. Thus, antibodies that block the binding of HSV to TR2 are useful in the treatment and prevention of Herpes simplex infections.

In highly preferred embodiments, antibodies of the invention that bind a TR2 polypeptide and inhibit entry of Herpes Simplex virus (HSV) into TR2 expressing cells are used to treat, prevent or ameliorate HSV infection. In specific embodiments, antibodies of the present invention are used to prevent, treat, diagnose, or inhibit the progression of HSV infection and other related disorders.

Other viral infections that may be treated, prevented, diagnosed, or inhibited with antibodies of the present invention include, but are not limited to, Arbovirus, Adenoviridae, Arenaviridae, Arterivirus, Birnaviridae, Bunyaviridae, Caliciviridae, Circoviridae, Coronaviridae, Dengue, EBV, HIV, Flaviviridae, Hepadnaviridae (Hepatitis), Herpesviridae (such as, Cytomegalovirus, Herpes Simplex, Herpes Zoster), Mononegavirus (e.g., Paramyxoviridae, Morbillivirus, Rhabdoviridae), Orthomyxoviridae (e.g., Influenza A, Influenza B, and parainfluenza), Papiloma virus, Papovaviridae, Parvoviridae, Picornaviridae, Poxviridae (such as Smallpox or Vaccinia), Reoviridae (e.g., Rotavirus), Retroviridae (HTLV-I, HTLV-II, Lentivirus), and Togaviridae (e.g., Rubivirus). Viruses falling within these families can cause a variety of diseases or symptoms, including, but not limited to: arthritis, bronchiollitis, respiratory syncytial virus, encephalitis, eye infections (e.g., conjunctivitis, keratitis), chronic fatigue syndrome, hepatitis (A, B, C, E, Chronic Active, Delta), Japanese B encephalitis, Junin, Chikungunya, Rift Valley fever, yellow fever, meningitis, opportunistic infections (e.g., AIDS), pneumonia, Burkitt's Lymphoma, chickenpox, hemorrhagic fever, Measles, Mumps, Parainfluenza, Rabies, the common cold, Polio, leukemia, Rubella, sexually transmitted diseases, skin diseases (e.g., Kaposi's, warts), and viremia.

TR2 has been shown to mediate HSV-1 entry into cells. Montgomery, R. et al., Eur. Cytokine Newt. 7:159 (1996). Further, antibodies specific for the extracellular domain of TR2 have been shown to block HSV-1 entry into cells. Therefore, the present invention includes antibodies capable of preventing TR2 mediated viral entry into TR2-expressing cells. Thus, antibodies that block the binding of HSV to TR2 are useful in the treatment and prevention of Herpes simplex infections.

Therapeutic Uses of Antibodies for Treating Cancers

In highly preferred embodiments, antibodies of the invention, e.g., antibodies of the invention that bind a TR2 polypeptide and inhibit proliferation of TR2 expressing cells, are used to treat, prevent or ameliorate cancer. In specific embodiments, antibodies of the invention are used to inhibit the progression or metastasis of cancers and other related disorders. Cancers and related disorders, include, but are not limited to, colon cancer, cervical cancer, leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

Additional Therapeutic Uses of Antibodies

In another embodiment, the invention provides methods and compositions for stimulating the differentiation and/or activation of TR2 expressing cells (e.g. T-cells), comprising, or alternatively consisting of, administering to an animal in which such differentiation and/or activation of TR2 expressing cells is desired, antibody or antibody compositions of the invention (e.g., antibody fragments and variants, antibody mixtures, antibody multimers, fusion proteins of the invention, and antibodies in combination with other therapeutic compounds such as antiviral agents) in an amount effective to stimulate differentiation and/or activation of TR2 expressing cells.

In another embodiment, the invention provides methods and compositions for inhibiting the proliferation, differentiation, and/or HSV infection of TR2 expressing cells, comprising, or alternatively consisting of, administering to an animal in which such inhibition of proliferation, differentiation, and/or HSV infection of TR2 expressing cells is desired, antibody or antibody compositions of the invention (e.g., antibody fragments and variants, antibody mixtures, antibody multimers, fusion proteins of the invention, and antibodies in combination with other therapeutic compounds such as antiviral agents) in an amount effective to inhibit the proliferation, differentiation, and/or HSV infection of TR2 expressing cells.

In one aspect, the present invention is directed to a method for inhibiting proliferation, differentiation, and/or HSV infection induced by a TR2 ligand (e.g., LIGHT and/or LTα), which involves administering to a cell which expresses a TR2 polypeptide an effective amount of an antibody of the invention, preferably an antagonistic anti-TR2, anti-TR2-SV1, and/or anti-TR2-SV2 antibody, capable of inhibiting or decreasing TR2 mediated signaling, especially TR2, TR2-SV1, and TR2-SV2 mediated signalling. Preferably, TR2 mediated signaling is decreased or inhibited by an antibody of the invention to treat a disease wherein decreased proliferation, differentiation, or decreased cytokine and adhesion molecule expression is exhibited.

In a further aspect, the present invention is directed to a method for stimulating proliferation and/or differentiation induced by a TR2 ligand (e.g., LIGHT and/or LTα), which involves administering to a cell which expresses a TR2 polypeptide, an effective amount of an antibody of the invention, preferably an agonistic anti-TR2, anti-TR2-SV1, and/or anti-TR2-SV2 antibody, capable of increasing TR2 mediated signaling, especially TR2, TR2-SV1, and TR2-SV2 mediated signalling. Preferably, TR2 mediated signaling is increased to treat a disease wherein decreased proliferation, differentiation, or NFKB expression is exhibited.

By TR2 “agonist” is intended naturally occurring and synthetic compounds capable of enhancing or potentiating TR2 mediated biological activities such as cellular proliferation and/or differentiation. By TR2 “antagonist” is intended naturally occurring and synthetic compounds capable of inhibiting TR2 mediated biological activities such as cellular proliferation, differentiation, and/or HSV entry. Whether any candidate “agonist” or “antagonist” of the present invention can enhance or inhibit, respectively, proliferation, differentiation, and/or HSV entry can be determined using art-known TNF-family ligand/receptor cellular response assays, including those described in more detail below.

The antibodies of the invention can be used to treat, ameliorate or prevent diseases, disorders or conditions associated with aberrant expression and/or activity of TR2 or TR2 ligand, including, but not limited to, any one or more of the diseases, disorders, or conditions described herein. The treatment and/or prevention of diseases, disorders, or conditions associated with aberrant TR2 expression and/or activity or aberrant TR2 ligand expression and/or activity includes, but is not limited to, alleviating symptoms associated with those diseases, disorders or conditions. Antibodies of the invention may be provided in pharmaceutically acceptable compositions as known in the art or as described herein.

Further, antibodies of the present invention (including molecules comprising, or alternatively consisting of, antibody fragments or variants thereof) which inhibit TR2-mediated biological activities (e.g., the induction of proliferation or differentation in TR2 expressing cells) can be administered to an animal to treat, prevent or ameliorate a disease or disorder described herein, particularly cancers and other hyperproliferative disorders, HSV infection, autoimmune disorders, as well as graft rejection and graft-versus-host disease. These antibodies may inhibit either all or a subset of the biological activities of TR2, for example, by inducing a conformational change in TR2. In a specific embodiment, an antibody of the present invention that decreases TR2 activity by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% relative to TR2 activity in absence of the antibody is administered to an animal to treat, prevent or ameliorate a disease or disorder. In another embodiment, a combination of antibodies, a combination of antibody fragments, a combination of antibody variants, or a combination of antibodies, antibody fragments and/or antibody variants that decrease TR2 activity by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% relative to TR2 activity in absence of the said antibodies or antibody fragments and/or antibody variants is administered to an animal to treat, prevent or ameliorate a disease or disorder.

Further, antibodies of the present invention (including molecules comprising, or alternatively consisting of, antibody fragments or variants thereof) which inhibit TR2-mediated biological activities (e.g., the induction of profileration in TR2 expressing cells) can be administered to an animal to treat, prevent or ameliorate a disease or disorder associated with aberrant TR2 expression, excessive TR2 function, aberrant TR2 ligand expression, or excessive TR2 ligand function. These antibodies may inhibit either all or a subset of the biological activities of TR2, for example, by inducing a conformational change in TR2. In a specific embodiment, an antibody of the present invention that decreases TR2 activity by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% relative to TR2 activity in absence of the antibody is administered to an animal to treat, prevent or ameliorate a disease or disorder associated with aberrant TR2 expression, excessive TR2 function, aberrant TR2 ligand expression, or excessive TR2 ligand function. In another embodiment, a combination of antibodies, a combination of antibody fragments, a combination of antibody variants, or a combination of antibodies, antibody fragments and/or antibody variants that increase TR2 activity by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%relative to TR2 activity in absence of the said antibodies or antibody fragments and/or antibody variants is administered to an animal to treat, prevent or ameliorate a disease or disorder associated with aberrant TR2 expression or excessive TR2 function or aberrant TR2 ligand expression or excessive TR2 ligand function.

Antibodies of the present invention (including molecules comprising, or alternatively consisting of, antibody fragments or variants thereof) that function as agonists or antagonists of TR2, preferably of TR2 signal transduction, can be administered to an animal to treat, prevent or ameliorate a disease or disorder associated with aberrant TR2 expression, TR2 function, TR2 ligand expression, or TR2 ligand function. For example, antibodies of the invention which mimic the action of TR2 binding to, in full or in part, (e.g. antibodies that act as TR2 agonists), may be administered to an animal to treat, prevent or ameliorate a disease or disorder associated with aberrant TR2 expression, insufficient TR2 function, aberrant TR2 ligand expression, or insufficient TR2 ligand function. As an alternative example, antibodies of the invention which disrupt or prevent the interaction between TR2 and its ligand, may be administered to an animal to treat, prevent or ameliorate a disease or disorder associated with aberrant TR2 expression, excessive TR2 function, aberrant TR2 ligand expression, or excessive TR2 ligand function. Antibodies of the invention which do not prevent TR2 from binding its ligand but stimulate or upregulate TR2 signal transduction can be administered to an animal to treat, prevent or ameliorate a disease or disorder associated with aberrant TR2 expression, insufficient TR2 function, aberrant TR2 ligand expression, or insufficient TR2 ligand function. The ability of an antibody of the invention to enhance, inhibit, upregulate or downregulate TR2 signal transduction may be determined by techniques described herein or otherwise known in the art. For example, TR2-induced receptor activation and the activation of signaling molecules can be determined by detecting the association of adaptor proteins such as TRAF with the TR2 proteins, by immunoprecipitation followed by western blot analysis (for example, as described herein).

Further, antibodies of the present invention (including molecules comprising, or alternatively consisting of, antibody fragments or variants thereof) which stimulate or increase TR2-mediated biological activities (e.g., the induction of proliferation in TR2 expressing cells) can be administered to an animal to treat, prevent or ameliorate a disease or disorder associated with aberrant TR2 expression, insufficient TR2 function, aberrant TR2 ligand expression, or insufficientTR2 ligand function. These antibodies may stimulate or increase either all or a subset of the biological activities of TR2, for example, by inducing a conformational change in TR2. In a specific embodiment, an antibody of the present invention that increases TR2 activity by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least two-fold, at least three-fold, at least four fold, at least five fold, at least ten-fold, at least twenty-fold, at least fifty-fold, or at least one hundred-fold relative to TR2 activity in absence of the antibody is administered to an animal to treat, prevent or ameliorate a disease or disorder associated with aberrant TR2 expression, insufficient TR2 function, aberrant TR2 ligand expression, or insufficient TR2 ligand function. In another embodiment, a combination of antibodies, a combination of antibody fragments, a combination of antibody variants, or a combination of antibodies, antibody fragments and/or antibody variants that increase TR2 activity by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least two-fold, at least three-fold, at least four fold, at least five fold, at least ten-fold, at least twenty-fold, at least fifty-fold, or at least one hundred-fold relative to TR2 activity in absence of the said antibodies or antibody fragments and/or antibody variants is administered to an animal to treat, prevent or ameliorate a disease or disorder associated with aberrant TR2 expression or lack of TR2 function or aberrant TR2 ligand expression or lack of TR2 ligand function. In a specific embodiment, antibodies of the invention that stimulatre TR2 mediated biological activities are used to treat an immunodeficiency.

Therapeutic or pharmaceutical compositions of the invention, may be administered to treat, prevent, or ameliorate immunodeficiencies and/or conditions associated therewith. Immunodeficiencies and related disorders include, but are not limited to, severe combined immunodeficiency (SCID)-X linked, SCID-autosomal, adenosine deaminase deficiency (ADA deficiency), X-linked agammaglobulinemia (XLA), Bruton's disease, congenital agammaglobulinemia, X-linked infantile agammaglobulinemia, acquired agammaglobulinemia, adult onset agammaglobulinemia, late-onset agammaglobulinemia, dysgammaglobulinemia, hypogammaglobulinemia, transient hypogammaglobulinemia of infancy, unspecified hypogammaglobulinemia, agammaglobulinemia, common variable immunodeficiency (CVID) (acquired), Wiskott-Aldrich Syndrome (WAS), X-linked immunodeficiency with hyper IgM, non X-linked immunodeficiency with hyper IgM, selective IgA deficiency, IgG subclass deficiency (with or without IgA deficiency), antibody deficiency with normal or elevated Igs, immunodeficiency with thymoma, Ig heavy chain deletions, kappa chain deficiency, B cell lymphoproliferative disorder (BLPD), selective IgM immunodeficiency, recessive agammaglobulinemia (Swiss type), reticular dysgenesis, neonatal neutropenia, autoimmune neutropenia, severe congenital leukopenia, thymic alymphoplasia-aplasia or dysplasia with immunodeficiency, ataxia-telangiectasia, short limbed dwarfism, X-linked lymphoproliferative syndrome (XLP), Nezelof syndrome-combined immunodeficiency with Igs, purine nucleoside phosphorylase deficiency (PNP), MHC Class II deficiency (Bare Lymphocyte Syndrome) and severe combined immunodeficiency. In preferred embodiments, TR2 agonistic antibodies, (e.g., antibodies that bind one or more TR2 polypeptides and transduce the biological signal that results in cellular profileration or differentiation) are used to treat the diseases and disorders listed above.

Therapeutic or pharmaceutical compositions of the invention, may also be administered to treat, prevent, or ameliorate organ rejection or graft-versus-host disease (GVHD) and/or conditions associated therewith. Organ rejection occurs by host immune cell destruction of the transplanted tissue through an immune response. Similarly, an immune response is also involved in GVHD, but, in this case, the foreign transplanted immune cells destroy the host tissues. Thus, the administration of antibodies of the invention, (e.g., antagonistic anti-TR2 antibodies of the invention), may be an effective therapy in preventing organ rejection or GVHD.

In another embodiments, antibodies and antibody compositions of the present invention are used to treat, prevent, or ameliorate diseases associated with increased apoptosis including, but not limited to, AIDS, neurodegenerative disorders (such as Alzheimer's disease, Parkinson's disease, Amyotrophic lateral sclerosis, Retinitis pigmentosa, Cerebellar degeneration), brain tumor or prion associated disease); autoimmune disorders (such as, multiple sclerosis, Rheumatoid Arthritis, Sjogren's syndrome, Hashimoto's thyroiditis, biliary cirrhosis, Behcet's disease, Crohn's disease, polymyositis, systemic lupus erythematosus and immune-related glomerulonephritis and rheumatoid arthritis) myelodysplastic syndromes (such as aplastic anemia), graft v. host disease, ischemic injury (such as that caused by myocardial infarction, stroke and reperfusion injury), liver injury (e.g., hepatitis related liver injury, ischemia/reperfusion injury, cholestosis (bile duct injury) and liver cancer); toxin-induced liver disease (such as that caused by alcohol), septic shock, cachexia and anorexia.

In additional embodiments, antibodies of the present invention, particularly agonistic anti-TR2 antibodies, are administered in combination with other stimulators of T cell differentiation.

Suitable agents, which also block binding of TR2 ligand (e.g., LIGHT or LTα) to TR2 that may be administered with the antibodies of the present invention include, but are not limited to, soluble TR2 polypeptides (e.g., TR2-SV1 (SEQ ID NO:4); soluble forms of TR2; a soluble form of TR6, as described in WO 98/30694, W)2001/52028, and WO2002/18622, which are hereby incorporated by reference in their entirety; soluble forms of LTβR, as described in WO2000/71150, which is hereby incorporated by reference in their entirety; and TR2-SV2 (SEQ ID NO:6)); multimeric forms of soluble TR2 polypeptides; and TR2 antibodies that bind TR2 without transducing the biological signal that results in proliferation and/or differentiation, anti-TR2 antibodies that block binding of TR2 to one or more TR2 proteins, and muteins of TR2 that bind TR2 proteins but do not transduce the biological signal that results in proliferation and/or differentiation.

Antibodies and antibody compositions of the invention may be useful for treating inflammatory diseases, such as rheumatoid arthritis, osteoarthritis, psoriasis, septicemia, and inflammatory bowel disease.

In addition, due to lymphoblast expression of TR2 polypeptides, antibodies and antibody compositions of the invention may be used to treat this form of cancer. Further, antibodies and antibody compositions of the invention may be used to treat various chronic and acute forms of inflammation such as rheumatoid arthritis, osteoarthritis, psoriasis, septicemia, and inflammatory bowel disease.

Antibodies and antibody compositions of the invention are useful in the diagnosis and treatment or prevention of a wide range of diseases and/or conditions. Such diseases and conditions include, but are not limited to, cancer (e.g., immune cell related cancers, breast cancer, prostate cancer, ovarian cancer, follicular lymphoma, cancer associated with mutation or alteration of p53, brain tumor, bladder cancer, uterocervical cancer, colon cancer, colorectal cancer, non-small cell carcinoma of the lung, small cell carcinoma of the lung, stomach cancer, etc.), lymphoproliferative disorders (e.g., lymphadenopathy), microbial (e.g., viral, bacterial, etc.) infection (e.g., HIV-1 infection, HIV-2 infection, herpesvirus infection (including, but not limited to, HSV-1, HSV-2, CMV, VZV, HHV-6, HHV-7, EBV), adenovirus infection, poxvirus infection, human papilloma virus infection, hepatitis infection (e.g., HAV, HBV, HCV, etc.), Helicobacter pylori infection, invasive Staphylococcia, etc.), parasitic infection, nephritis, bone disease (e.g., osteoporosis), atherosclerosis, pain, cardiovascular disorders (e.g., neovascularization, hypovascularization or reduced circulation (e.g., ischemic disease (e.g., myocardial infarction, stroke, etc.))), AIDS, allergy, inflammation, neurodegenerative disease (e.g., Alzheimer's disease, Parkinson's disease, amyotrophic lateral sclerosis, pigmentary retinitis, cerebellar degeneration, etc.), graft rejection (acute and chronic), graft vs. host disease, diseases due to osteomyelodysplasia (e.g., aplastic anemia, etc.), joint tissue destruction in rheumatism, liver disease (e.g., acute and chronic hepatitis, liver injury, and cirrhosis), autoimmune disease (e.g., multiple sclerosis, rheumatoid arthritis, systemic lupus erythematosus, autoimmune lymphoproliferative syndrome (ALPS), immune complex glomerulonephritis, autoimmune diabetes, autoimmune thrombocytopenic purpura, Grave's disease, Hashimoto's thyroiditis, etc.), cardiomyopathy (e.g., dilated cardiomyopathy), diabetes, diabetic complications (e.g., diabetic nephropathy, diabetic neuropathy, diabetic retinopathy), influenza, asthma, psoriasis, glomerulonephritis, septic shock, and ulcerative colitis.

Antibodies and antibody compositions of the invention are also useful as an adjuvant to enhance immune responsiveness to specific antigen, such as in anti-viral immune responses.

More generally, antibodies and antibody compositions of the invention are useful in regulating (i.e., elevating or reducing) immune response. For example, antibodies and antibody compositions of the invention may be useful in preparation or recovery from surgery, trauma, radiation therapy, chemotherapy, and transplantation, or may be used to boost immune response and/or recovery in the elderly and immunocompromised individuals. Alternatively, antibodies and antibody compositions of the invention are useful as immunosuppressive agents, for example in the treatment or prevention of autoimmune disorders. In specific embodiments, antibodies and antibody compositions of the invention are used to treat or prevent chronic inflammatory, allergic or autoimmune conditions, such as those described herein or are otherwise known in the art.

In rejection of an allograft, the immune system of the recipient animal has not previously been primed to respond because the immune system for the most part is only primed by environmental antigens. Tissues from other members of the same species have not been presented in the same way that, for example, viruses and bacteria have been presented. In the case of allograft rejection, immunosuppressive regimens are designed to prevent the immune system from reaching the effector stage. However, the immune profile of xenograft rejection may resemble disease recurrence more that allograft rejection. In the case of disease recurrence, the immune system has already been activated, as evidenced by destruction of the native islet cells. Therefore, in disease recurrence the immune system is already at the effector stage. Antibodies of the present invention (e.g., antagonistic antibodies of the invention) may be able to suppress the immune response to both allografts and xenografts. Antagonists of the invention can further be used in the treatment of Inflammatory Bowel-Disease.

Therapeutic/Prophylactic Compositions and Administration

The invention provides methods of treatment, inhibition and prophylaxis by administration to a subject of an effective amount of antibody (or fragment or variant thereof) or pharmaceutical composition of the invention, preferably an antibody of the invention. In a preferred aspect, an antibody or fragment or variant thereof is substantially purified (i.e., substantially free from substances that limit its effect or produce undesired side-effects). The subject is preferably an animal, including but not limited to, animals such as cows, pigs, horses, chickens, cats, dogs, etc., and is preferably a mammal, and most preferably a human.

Formulations and methods of administration that can be employed when the compound comprises a nucleic acid or an immunoglobulin are described above; additional appropriate formulations and routes of administration can be selected from among those described herein below.

Various delivery systems are known and can be used to administer antibody or fragment or variant thereof of the invention, e.g., encapsulation in liposomes, microparticles, microcapsules, recombinant cells capable of expressing the antibody or antibody fragment, receptor-mediated endocytosis (see, e.g., Wu and Wu, J. Biol. Chem. 262:4429-4432 (1987)), construction of a nucleic acid as part of a retroviral or other vector, etc. Methods of introduction include, but are not limited to, intradermal, intramuscular, intraperitoneal, intravenous, subcutaneous, intranasal, epidural, and oral routes. The compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, of the invention, care must be taken to use materials to which the protein does not absorb.

In another embodiment, the composition can be delivered in a vesicle, in particular a liposome (see Langer, Science 249:1527-1535 (1990); Treat et al., in Liposomes in the Therapy of Infectious Disease and Cancer, Lopez-Berestein and Fidler (eds.), Liss, New York, pp. 353- 365 (1989); Lopez-Berestein, ibid., pp. 3 17-327; see generally ibid.).

In yet another embodiment, the composition can be delivered in a controlled release system. In one embodiment, a pump may be used (see Langer, supra; Sefton, CRC Crit. Ref. Biomed. Eng. 14:20 1 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, J., Macromol. Sci. Rev. Macromol. Chem. 23:71 (1983); see also Levy et al., Science 228:190 (1985); During et al., Ann. Neurol. 25:35 1 (1989); Howard et al., J.Neurosurg. 7 1:105 (1989)). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)).

Other controlled release systems are discussed in the review by Langer (Science 249:1527-1535 (1990)).

In a specific embodiment where the composition of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., Proc. Natl. Acad. Sci. USA 88:1864-1868 (1991)), etc. Alternatively, a nucleic acid can be introduced intracellularly and incorporated within host cell DNA for expression, by homologous recombination.

The present invention also provides pharmaceutical compositions. Such compositions comprise a therapeutically effective amount of an antibody or a fragment thereof, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the antibody or fragment thereof, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocamne to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The amount of the composition of the invention which will be effective in the treatment, inhibition and prevention of a disease or disorder associated with aberrant expression and/or activity of a polypeptide of the invention can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems.

For antibodies, the dosage administered to a patient is typically 0.1 mg/kg to 100 mg/kg of the patient's body weight. Preferably, the dosage administered to a patient is between 0.1 mg/kg and 20 mg/kg of the patient's body weight, more preferably 1 mg/kg to 10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of therapeutic or pharmaceutical compositions of the invention may be reduced by enhancing uptake and tissue penetration (e.g., into the brain) of the antibodies by modifications such as, for example, lipidation.

Generally, administration of products of a species origin or species reactivity (in the case of antibodies) that is the same species as that of the patient is preferred. Thus, in a preferred embodiment, human antibodies, fragments, or variants, (e.g., derivatives), or nucleic acids, are administered to a human patient for therapy or prophylaxis.

It is preferred to use high affinity and/or potent in vivo inhibiting and/or neutralizing antibodies of the invention (including molecules comprising, or alternatively consisting of, antibody fragments or variants thereof) that specifically bind to one or more TR2 proteins, or polynucleotides encoding antibodies that specifically bind to one or more TR2 proteins, for both immunoassays and therapy of disorders related to TR2 polynucleotides or polypeptides, including fragments thereof. Such antibodies will preferably have an affinity for TR2 polypeptides and/or TR2 polypeptide fragments. Preferred binding affinities include those with a dissociation constant or K_{D} of less than or equal to 5×10⁻² M, 10⁻² M, 5×10⁻³ M, 10⁻³ M, 5×10⁻⁴ M, 10⁻⁴ M, 5×10⁻⁵ M, or 10⁻⁵ M. More preferably, antibodies of the invention bind TR2 polypeptides or fragments or variants thereof with a dissociation constant or K_{D} less than or equal to 5×10⁻⁶ M, 10⁻⁶ M, 5×10⁻⁷ M, 10⁻⁷ M, 5×10⁻⁸ M, or 10⁻⁸ M. Even more preferably, antibodies of the invention bind TR2 polypeptides or fragments or variants thereof with a dissociation constant or K_{D} less than or equal to 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×⁻¹³M, 10⁻¹³ M, 5×10⁻¹⁴ M, 10⁻¹⁴ N, 5×10⁻¹⁵ M, or 10⁻¹⁵ M. In a preferred embodiment, antibodies of the invention inhibit proliferation, differentiation, and/or HSV infection of TR2 expressing cells. In an additional preferred embodiment, antibodies of the invention induce differentiation of TR2 expressing cells.

As discussed in more detail below, the antibodies of the present invention may be used either alone or in combination with other compositions. The antibodies may further be recombinantly fused to a heterologous polypeptide at the N- or C-terminus or chemically conjugated (including covalent and non-covalent conjugations) to polypeptides or other compositions. For example, antibodies of the present invention may be recombinantly fused or conjugated to molecules useful as labels in detection assays and effector molecules such as heterologous polypeptides, drugs, radionuclides, or toxins. See, e.g., PCT publications WO 92/08495; WO 91/14438; WO 89/12624; U.S. Pat. No. 5,314,995; and EP 396,387.

The antibody and antibody compositions of the invention may be administered alone or in combination with other therapeutic agents, including but not limited to chemotherapeutic agents, antibiotics, antivirals, anti-retroviral agents, steroidal and non-steroidal anti-inflammatories, conventional immunotherapeutic agents and cytokines. Combinations may be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents given first, followed by the second.

Combination Therapies with Anti-TR2 Antibodies, Immunomodulatory Agents, Antiviral Drugs, and/or Chemotherapeutic Agents

Anti-TR2 antibodies may be administered in combination with other anti-TR2 antibodies, TR2, and/or chemotherapeutics.

In specific embodiments, an antibody of the invention that specifically binds TR2 is used or administered in combination with a second antibody that specifically binds TR2-SV2. In another embodiment, the antibodies specific for TR2 and TR2-SV2 are antagonistic antibodies that inhibit proliferation, differentiation, and/or HSV infection of TR2 expressing cells. In a specific embodiment, the combination of anti-TR2 treatment and anti-TR2-SV2 treatment inhibits more proliferation, differentiation, and/or HSV infection of TR2 expressing cells than either anti-TR2 antibody treatment or anti-TR2-SV2 antibody treatment alone. In another embodiment, the antibodies specific for TR2 and TR2-SV2 are agonistic antibodies that stimulate differentiation and/or activation of TR2 expressing cells (e.g. T-cells). In a specific embodiment, the combination of anti-TR2 treatment and anti-TR2-SV2 treatment inhibits more proliferation, differentiation, and/or HSV infection or sitmulates more differentiation and/or activation of TR2 expressing cells than either anti-TR2 antibody treatment or anti-TR2-SV2 antibody treatment alone. The anti-TR2 and anti-TR2-SV2 antibodies can be administered either simultaneously, sequentially, or a combination of simultaneous or sequential administration throughout the dosage regimen. In another specific embodiment anti-TR2 and anti-TR2-SV2 antibodies are used or administered in combination with a chemotherapeutic drug, antiviral drug, and/or immunomodulatory drug. In a particular embodiment, the synergistic inhibition of proliferation, differentiation, and/or HSV infection or stimulation of differentiation resulting from anti-TR2 and anti-TR2-SV2 antibody treatment, is more evident or more pronounced when the anti-TR2 and anti-TR2-SV2 antibodies are used or administered in combination with a chemotherapeutic agent, antiviral drug, immunomodulatory drug, and/or a cross-linking reagent.

In specific embodiments, an antibody of the invention that specifically binds TR2 is used or administered in combination with a second antibody that specifically binds TR2-SV1. In another embodiment, the antibodies specific for TR2 and TR2-SV1 are antagonistic antibodies that inhibit proliferation, differentiation, and/or HSV infection of TR2 expressing cells. In a specific embodiment, the combination of anti-TR2 treatment and anti-TR2-SV1 treatment inhibits more proliferation, differentiation, and/or HSV infection of TR2 expressing cells than either anti-TR2 antibody treatment or anti-TR2-SV1 antibody treatment alone. In another embodiment, the antibodies specific for TR2 and TR2-SV1 are agonistic antibodies that stimulate differentiation and/or activation of TR2 expressing cells (e.g. T-cells). In a specific embodiment, the combination of anti-TR2 treatment and anti-TR2-SV1 treatment stimulates more activation and/or differentiation of TR2 expressing cells than either anti-TR2 antibody treatment or anti-TR2-SV1 antibody treatment alone. The anti-TR2 and anti-TR2-SV1 antibodies can be administered either simultaneously, sequentially, or a combination of simultaneous or sequential administration throughout the dosage regimen. In another specific embodiment anti-TR2 and anti-TR2-SV1 antibodies are used or administered in combination with a chemotherapeutic drug, antiviral drug, and/or immunomodulatory drug. In a particular embodiment, the synergistic inhibition of proliferation, differentiation, and/or HSV infection or stimulation of differentiation and/or activation resulting from anti-TR2 and anti-TR2-SV1 antibody treatment, is more evident or more pronounced when the anti-TR2 and anti-TR2-SV1 antibodies are used or administered in combination with a chemotherapeutic agent, antiviral drug, immunomodulatory drug, and/or a cross-linking reagent.

In other embodiments, anti-TR2-SV2 antibodies may be administered in combination with anti-TR2-SV1 antibodies. In preferred embodiments, the anti-TR2-SV1 and/or anti-TR2-SV2 antibodies inhibit the binding of TR2 ligand to TR2-SV1 and TR2-SV2, respectively.

In additional embodiments, anti-TR2 antibodies of the present invention may be administered in combination with a soluble form of other TNF-family receptors which include, but are not limited to, TL5, LTα, TR6, and LTβR.

In other embodiments, antibody compositions of the invention may be administered in combination with anti-opportunistic infection agents. Anti-opportunistic agents that may be administered in combination with the albumin fusion proteins and/or polynucleotides of the invention, include, but are not limited to, TRIMETHOPRIM-SULFAMETHOXAZOLE™, DAPSONE™, PENTAMIDINE™, ATOVAQUONE™, ISONIAZID™, RIFAMPIN™, PYRAZINAMIDE™, ETHAMBUTOL™, RIFABUTIN™, CLARITHROMYCIM™, AZITHROMYCIN™, GANCICLOVIR™, FOSCARNET™, CIDOFOVIR™, FLUCONAZOLE™, ITRACONAZOLE™, KETOCONAZOLE™, ACYCLOVIR™, FAMCICOLVIR™, PYRIMETHAMINE™, LEUCOVORIN™, NEUPOGEN™ (filgrastim/G-CSF), and LEUKINE™ (sargramostim/GM-CSF).

In a specific embodiment, antibodies of the invention are used in any combination with ACYCLOVIR™ and/or FAMCICOLVIR™ to prophylactically treat or prevent an opportunistic herpes simplex virus type I and/or type II infection.

In a preferred embodiment, compositions of the invention are administered in combination with a chemotherapeutic agent. Chemotherapeutic agents that may be administered with the compositions of the invention include, but are not limited to, antibiotic derivatives (e.g., doxorubicin (adriamycin), bleomycin, daunorubicin, and dactinomycin); antiestrogens (e.g., tamoxifen); antimetabolites (e.g., fluorouracil, 5-FU, methotrexate, floxuridine, interferon alpha-2b, glutamic acid, plicamycin, mercaptopurine, and 6-thioguanine); cytotoxic agents (e.g., carmustine, BCNU, lomustine, CCNU, cytosine arabinoside, cyclophosphamide, estramustine, hydroxyurea, procarbazine, mitomycin, busulfan, cis-platin, and vincristine sulfate); hormones (e.g., medroxyprogesterone, estramustine phosphate sodium, ethinyl estradiol, estradiol, megestrol acetate, methyltestosterone, diethylstilbestrol diphosphate, chlorotrianisene, and testolactone); nitrogen mustard derivatives (e.g., mephalen, chorambucil, mechlorethamine (nitrogen mustard) and thiotepa); steroids and combinations (e.g., bethamethasone sodium phosphate); and others (e.g., dicarbazine, asparaginase, mitotane, vincristine sulfate, vinblastine sulfate, etoposide, Topotecan, 5-Fluorouracil, paclitaxel (Taxol), Cisplatin, Cytarabine, and IFN-gamma, irinotecan (Camptosar, CPT-11), and gemcitabine (GEMZAR™)).

In a specific embodiment, antibody and antibody compositions of the invention are administered in combination with steroids, cyclosporine, cyclosporine analogs, cyclophosphamide methylprednisone, prednisone, azathioprine, FK-506, 15-deoxyspergualin, and other immunosuppressive agents that act by suppressing the function of responding T cells. Other immunosuppressive agents that may be administered in combination with the compositions of the invention include, but are not limited to, prednisolone, methotrexate, thalidomide, methoxsalen, rapamycin, leflunomide, mizoribine (BREDININ™), brequinar, deoxyspergualin, and azaspirane (SKF 105685), ORTHOCLONE OKT® 3 (muromonab-CD3), SANDIMMUNE™, NEORAL™, SANGDYA™ (cyclosporine), PROGRAF® (FK506, tacrolimus), CELLCEPT® (mycophenolate motefil, of which the active metabolite is mycophenolic acid), IMURAN™ (azathioprine), glucocorticosteroids, adrenocortical steroids such as DELTASONE™ (prednisone) and HYDELTRASOL™ (prednisolone), FOLEX™ and MEXATE™ (methotrxate), OXSORALEN-ULTRA™ (methoxsalen) and RAPAMUNE™ (sirolimus). In a specific embodiment, immunosuppressants may be used to prevent rejection of organ or bone marrow transplantation.

In other embodiments, the compositions of the invention are administered in combination with immunestimulants including, but are not limited to, levamisole (e.g., ERGAMISOL™), isoprinosine (e.g. INOSIPLEX™), interferons (e.g. interferon alpha), and interleukins (e.g., IL-2).

In one embodiment, the compositions of the invention are administered in combination with other members of the TNF family or antibodies specific for TNF receptor family members. TNF, TNF-related or TNF-like molecules that may be administered with the compositions of the invention include, but are not limited to, soluble forms of TNF-alpha, lymphotoxin-alpha (LT-alpha, also known as TNF-beta), LT-beta (found in complex heterotrimer LT-alpha2-beta), OPGL, FasL, CD27L, CD30L, CD40L, 4-1BBL, DcR3, OX40L, TNF-gamma (International Publication No. WO 96/14328), AIM-II (International Publication No. WO 97/34911), APRIL (J. Exp. Med. 188(6):1185-1190), endokine-alpha (International Publication No. WO 98/07880), TR6 (International Publication No. WO 98/30694), OPG, and neutrokine-alpha (International Publication No. WO 98/18921, OX40, and nerve growth factor (NGF), and soluble forms of Fas, CD30, CD27, CD40 and 4-1BB, TR2 (International Publication No. WO 96/34095), DR3 (International Publication No. WO 97/35904), TR6 (International Publication No. WO 98/30694), TRANK, TR9 (International Publication No. WO 98/56892),TR10 (International Publication No. WO 98/54202),312C2 (International Publication No. WO 98/06842), and TR12, and soluble forms CD154, CD70, and CD 153.

Additional Combination Therapies

The antibodies of the invention (including molecules comprising, or alternatively consisting of, antibody fragments or variants thereof) may be administered alone or in combination with other therapeutic or prophylactic regimens (e.g., radiation therapy, chemotherapy, hormonal therapy, immunotherapy, anti-tumor agents, anti-angiogenesis and anti-inflammatory agents). Such combinatorial therapy may be administered sequentially and/or concomitantly.

The invention also encompasses combining the polynucleotides and/or polypeptides of the invention (and/or agonists or antagonists thereof) with other proposed or conventional hematopoietic therapies. Thus, for example, the polynucleotides and/or polypeptides of the invention (and/or agonists or antagonists thereof) can be combined with compounds that singly exhibit erythropoietic stimulatory effects, such as erythropoietin, testosterone, progenitor cell stimulators, insulin-like growth factor, prostaglandins, serotonin, cyclic AMP, prolactin, and triiodothyzonine. Also encompassed are combinations of the antibody and antibody compositions of the invention with compounds generally used to treat aplastic anemia, such as, for example, methenolene, stanozolol, and nandrolone; to treat iron-deficiency anemia, such as, for example, iron preparations; to treat malignant anemia, such as, for example, vitamin B₁₂ and/or folic acid; and to treat hemolytic anemia, such as, for example, adrenocortical steroids, e.g., corticoids. See e.g., Resegotti et al., Panminerva Medica, 23:243-248 (1981); Kurtz, FEBS Letters, 14a:105-108 (1982); McGonigle et al., Kidney Int., 25:437-444 (1984); and Pavlovic-Kantera, Expt. Hematol., 8(supp. 8) 283-291 (1980), the contents of each of which are hereby incorporated by reference in their entireties.

Compounds that enhance the effects of or synergize with erythropoietin are also useful as adjuvants herein, and include but are not limited to, adrenergic agonists, thyroid hormones, androgens, hepatic erythropoietic factors, erythrotropins, and erythrogenins, See for e.g., Dunn, “Current Concepts in Erythropoiesis”, John Wiley and Sons (Chichester, England, 1983); Kalmani, Kidney Int., 22:383-391 (1982); Shahidi, New Eng. J. Med., 289:72-80 (1973); Urabe et al., J. Exp. Med., 149:1314-1325 (1979); Billat et al., Expt. Hematol., 10:135-140 (1982); Naughton et al., Acta Haemat, 69:171-179 (1983); Cognote et al. in abstract 364, Proceedings 7th Intl. Cong. of Endocrinology (Quebec City, Quebec, Jul. 1-7, 1984); and Rothman et al., 1982, J. Surg. Oncol., 20:105-108 (1982). Methods for stimulating hematopoiesis comprise administering a hematopoietically effective amount (i.e., an amount which effects the formation of blood cells) of a pharmaceutical composition containing polynucleotides and/or poylpeptides of the invention (and/or agonists or antagonists thereof) to a patient. The polynucleotides and/or polypeptides of the invention and/or agonists or antagonists thereof is administered to the patient by any suitable technique, including but not limited to, parenteral, sublingual, topical, intrapulmonary and intranasal, and those techniques further discussed herein. The pharmaceutical composition optionally contains one or more members of the group consisting of erythropoietin, testosterone, progenitor cell stimulators, insulin-like growth factor, prostaglandins, serotonin, cyclic AMP, prolactin, triiodothyzonine, methenolene, stanozolol, and nandrolone, iron preparations, vitamin B₁₂, folic acid and/or adrenocortical steroids.

In an additional embodiment, the antibody and antibody compositions of the invention are administered in combination with hematopoietic growth factors. Hematopoietic growth factors that may be administered with the antibody and antibody compositions of the invention include, but are not limited to, LEUKINE™ (SARGRAMOSTIM™) and NEUPOGEN™ (FILGRASTIM™).

In an additional embodiment, the antibody and antibody compositions of the invention are administered alone or in combination with an anti-angiogenic agent(s). Anti-angiogenic agents that may be administered with the antibody and antibody compositions of the invention include, but are not limited to, Angiostatin (Entremed, Rockville, Md.), Troponin-1 (Boston Life Sciences, Boston, Mass.), anti-Invasive Factor, retinoic acid and derivatives thereof, paclitaxel (Taxol), Suramin, Tissue Inhibitor of Metalloproteinase-1, Tissue Inhibitor of Metalloproteinase-2, VEGI, Plasminogen Activator Inhibitor-1, Plasminogen Activator Inhibitor-2, and various forms of the lighter “d group” transition metals.

Lighter “d group” transition metals include, for example, vanadium, molybdenum, tungsten, titanium, niobium, and tantalum species. Such transition metal species may form transition metal complexes. Suitable complexes of the above-mentioned transition metal species include oxo transition metal complexes.

Representative examples of vanadium complexes include oxo vanadium complexes such as vanadate and vanadyl complexes. Suitable vanadate complexes include metavanadate and orthovanadate complexes such as, for example, ammonium metavanadate, sodium metavanadate, and sodium orthovanadate. Suitable vanadyl complexes include, for example, vanadyl acetylacetonate and vanadyl sulfate including vanadyl sulfate hydrates such as vanadyl sulfate mono- and trihydrates.

Representative examples of tungsten and molybdenum complexes also include oxo complexes. Suitable oxo tungsten complexes include tungstate and tungsten oxide complexes. Suitable tungstate complexes include ammonium tungstate, calcium tungstate, sodium tungstate dihydrate, and tungstic acid. Suitable tungsten oxides include tungsten (IV) oxide and tungsten (VI) oxide. Suitable oxo molybdenum complexes include molybdate, molybdenum oxide, and molybdenyl complexes. Suitable molybdate complexes include ammonium molybdate and its hydrates, sodium molybdate and its hydrates, and potassium molybdate and its hydrates. Suitable molybdenum oxides include molybdenum (VI) oxide, molybdenum (VI) oxide, and molybdic acid. Suitable molybdenyl complexes include, for example, molybdenyl acetylacetonate. Other suitable tungsten and molybdenum complexes include hydroxo derivatives derived from, for example, glycerol, tartaric acid, and sugars.

A wide variety of other anti-angiogenic factors may also be utilized within the context of the present invention. Representative examples include, but are not limited to, platelet factor 4; protamine sulphate; sulphated chitin derivatives (prepared from queen crab shells), (Murata et al., Cancer Res. 51:22-26, 1991); Sulphated Polysaccharide Peptidoglycan Complex (SP-PG) (the function of this compound may be enhanced by the presence of steroids such as estrogen, and tamoxifen citrate); Staurosporine; modulators of matrix metabolism, including for example, proline analogs, cishydroxyproline, d,L-3,4-dehydroproline, Thiaproline, alpha,alpha-dipyridyl, aminopropionitrile fumarate; 4-propyl-5-(4-pyridinyl)-2(3H)-oxazolone; Methotrexate; Mitoxantrone; Heparin; Interferons; 2 Macroglobulin-serum; ChIMP-3 (Pavloff et al., J. Bio. Chem. 267:17321-17326, 1992); Chymostatin (Tomkinson et al., Biochem J. 286:475-480, 1992); Cyclodextrin Tetradecasulfate; Eponemycin; Camptothecin; Fumagillin (Ingber et al., Nature 348:555-557, 1990); Gold Sodium Thiomalate (“GST”; Matsubara and Ziff, J. Clin. Invest. 79:1440-1446, 1987); anticollagenase-serum; alpha2-antiplasmin (Holmes et al., J. Biol. Chem. 262(4):1659-1664, 1987); Bisantrene (National Cancer Institute); Lobenzarit disodium (N-(2)-carboxyphenyl-4- chloroanthronilic acid disodium or “CCA”; (Takeuchi et al., Agents Actions 36:312-316, 1992); and metalloproteinase inhibitors such as BB94.

Additional anti-angiogenic factors that may also be utilized within the context of the present invention include Thalidomide, (Celgene, Warren, N.J.); Angiostatic steroid; AGM-1470 (H. Brem and J. Folkman J Pediatr. Surg. 28:445-51 (1993)); an integrin alpha v beta 3 antagonist (C. Storgard et al., J Clin. Invest. 103:47-54 (1999)); carboxynaminolmidazole; Carboxyamidotriazole (CAI) (National Cancer Institute, Bethesda, Md.); Conbretastatin A4 (CA4P) (OXiGENE, Boston, Mass.); Squalamine (Magainin Pharmaceuticals, Plymouth Meeting, Pa.); TNP-470, (Tap Pharmaceuticals, Deerfield, Ill.); ZD-0101 AstraZeneca (London, UK); APRA (CT2584); Benefin, Byrostatin-1 (SC359555); CGP-41251 (PKC 412); CM101; Dexrazoxane (ICRF187); DMXAA; Endostatin; Flavopridiol; Genestein; GTE; ImmTher; Iressa (ZD1839); Octreotide (Somatostatin); Panretin; Penacillamine; Photopoint; PI-88; Prinomastat (AG-3540) Purlytin; Suradista (FCE26644); Tamoxifen (Nolvadex); Tazarotene; Tetrathiomolybdate; Xeloda (Capecitabine); and 5-Fluorouracil.

Anti-angiogenic agents that may be administered in combination with the compounds of the invention may work through a variety of mechanisms including, but not limited to, inhibiting proteolysis of the extracellular matrix, blocking the function of endothelial cell-extracellular matrix adhesion molecules, by antagonizing the function of angiogenesis inducers such as growth factors, and inhibiting integrin receptors expressed on proliferating endothelial cells. Examples of anti-angiogenic inhibitors that interfere with extracellular matrix proteolysis and which may be administered in combination with the antibody and antibody compositions of the invention include, but are not limited to, AG-3540 (Agouron, La Jolla, Calif.), BAY-12-9566 (Bayer, West Haven , Conn.), BMS-275291 (Bristol Myers Squibb, Princeton, N.J.), CGS-27032A (Novartis, East Hanover, N.J.), Marimastat (British Biotech, Oxford, UK), and Metastat (Aetema, St-Foy, Quebec). Examples of anti-angiogenic inhibitors that act by blocking the function of endothelial cell-extracellular matrix adhesion molecules and which may be administered in combination with the antibody and antibody compositions of the invention include, but are not limited to, EMD-121974 (Merck KcgaA Darmstadt, Germany) and Vitaxin (Ixsys, La Jolla, Calif./Medimmune, Gaithersburg, Md.). Examples of anti-angiogenic agents that act by directly antagonizing or inhibiting angiogenesis inducers and which may be administered in combination with the antibody and antibody compositions of the invention include, but are not limited to, Angiozyme (Ribozyme, Boulder, Colo.), Anti-VEGF antibody (Genentech, S. San Francisco, Calif.), PTK-787/ZK-225846 (Novartis, Basel, Switzerland), SU-101 (Sugen, S. San Francisco, Calif.), SU-5416 (Sugen/Pharmacia Upjohn, Bridgewater, N.J.), and SU-6668 (Sugen). Other anti-angiogenic agents act to indirectly inhibit angiogenesis. Examples of indirect inhibitors of angiogenesis which may be administered in combination with the antibody and antibody compositions of the invention include, but are not limited to, IM-862 (Cytran, Kirkland, Wash.), Interferon-alpha, IL-12 (Roche, Nutley, N.J.), and Pentosan polysulfate (Georgetown University, Washington, D.C.).

In particular embodiments, the use of antibody and antibody compositions of the invention in combination with anti-angiogenic agents is contemplated for the treatment, prevention, and/or amelioration of cancers and other hyperproliferative disorders.

In certain embodiments, Therapeutics of the invention are administered in combination with antiretroviral agents, nucleoside/nucleotide reverse transcriptase inhibitors (NRTIs), non-nucleoside reverse transcriptase inhibitors (NNRTIs), and/or protease inhibitors (PIs). NRTIs that may be administered in combination with the Therapeutics of the invention, include, but are not limited to, RETROVIR™ (zidovudine/AZT), VIDEX™ (didanosine/ddI), HIVID™ (zalcitabine/ddC), ZERIT™ (stavudine/d4T), EPIVIR™ (lamivudine/3TC), and COMBIVIR™ (zidovudine/lamivudine). NNRTIs that may be administered in combination with the Therapeutics of the invention, include, but are not limited to, VIRAMNE™ (nevirapine), RESCRIPTOR™ (delavirdine), and SUSTIVA™ (efavirenz). Protease inhibitors that may be administered in combination with the Therapeutics of the invention, include, but are not limited to, CRIXIVAN™ (indinavir), NORVIR™ (ritonavir), INVIRASE™ (saquinavir), and VIRACEPT™ (nelfinavir). In a specific embodiment, antiretroviral agents, nucleoside reverse transcriptase inhibitors, non-nucleoside reverse transcriptase inhibitors, and/or protease inhibitors may be used in any combination with Therapeutics of the invention to treat AIDS and/or to prevent or treat HIV infection.

In a further embodiment, the antibody and antibody compositions of the invention are administered in combination with an antibiotic agent. Antibiotic agents that may be administered with the antibody and antibody compositions of the invention include, but are not limited to, amoxicillin, aminoglycosides, beta-lactam (glycopeptide), beta-lactamases, Clindamycin, chloramphenicol, cephalosporins, ciprofloxacin, ciprofloxacin, erythromycin, fluoroquinolones, macrolides, metronidazole, penicillins, quinolones, rifampin, streptomycin, sulfonamide, tetracyclines, trimethoprim, trimethoprim-sulfamthoxazole, and vancomycin.

In other embodiments, antibody and antibody compositions of the invention may be administered in combination with anti-opportunistic infection agents. In a specific embodiment, antibody and antibody compositions of the invention are used in any combination with TRIMETHOPRIM-SULFAMETHOXAZOLE™, DAPSONE™, PENTAMIDINE™, and/or ATOVAQUONE™ to prophylactically treat, prevent, and/or diagnose an opportunistic Pneumocystis carinii pneumonia infection. In another specific embodiment, antibody and antibody compositions of the invention are used in any combination with ISONIAZID™, RIFAMPIN™, PYRAZINAMIDE™, and/or ETHAMBUTOL™ to prophylactically treat, prevent, and/or diagnose an opportunistic Mycobacterium avium complex infection. In another specific embodiment, antibody and antibody compositions of the invention are used in any combination with RIFABUTIN™, CLARITHROMYCIN™, and/or AZITHROMYCIN™ to prophylactically treat, prevent, and/or diagnose an opportunistic Mycobacterium tuberculosis infection. In another specific embodiment, antibody and antibody compositions of the invention are used in any combination with GANCICLOVIR™, FOSCARNET™, and/or CIDOFOVIR™ to prophylactically treat, prevent, and/or diagnose an opportunistic cytomegalovirus infection. In another specific embodiment, antibody and antibody compositions of the invention are used in any combination with FLUCONAZOLE™, ITRACONAZOLE™, and/or KETOCONAZOLE™ to prophylactically treat, prevent, and/or diagnose an opportunistic fungal infection. In another specific embodiment, antibody and antibody compositions of the invention are used in any combination with ACYCLOVIR™ and/or FAMCICOLVIR™ to prophylactically treat, prevent, and/or diagnose an opportunistic herpes simplex virus type I and/or type II infection. In another specific embodiment, antibody and antibody compositions of the invention are used in any combination with PYRIMETHAMINE™ and/or LEUCOVORIN™ to prophylactically treat, prevent, and/or diagnose an opportunistic Toxoplasma gondii infection. In another specific embodiment, antibody and antibody compositions of the invention are used in any combination with LEUCOVORIN™ and/or NEUPOGEN™ to prophylactically treat, prevent, and/or diagnose an opportunistic bacterial infection.

In a preferred embodiment, the antibody and antibody compositions of the invention are administered in combination with steroid therapy. Steroids that may be administered in combination with the antibody and antibody compositions of the invention, include, but are not limited to, oral corticosteroids, prednisone, and methylprednisolone (e.g., IV methylprednisolone). In a specific embodiment, antibody and antibody compositions of the invention are administered in combination with prednisone.

In an additional embodiment, the antibody and antibody compositions of the invention are administered alone or in combination with an anti-inflammatory agent. Anti-inflammatory agents that may be administered with the antibody and antibody compositions of the invention include, but are not limited to, glucocorticoids and the nonsteroidal anti-inflammatories, aminoarylcarboxylic acid derivatives, arylacetic acid derivatives, arylbutyric acid derivatives, arylcarboxylic acids, arylpropionic acid derivatives, pyrazoles, pyrazolones, salicylic acid derivatives, thiazinecarboxamides, e-acetamidocaproic acid, S-adenosylmethionine, 3-amino-4-hydroxybutyric acid, amixetrine, bendazac, benzydamine, bucolome, difenpiramide, ditazol, emorfazone, guaiazulene, nabumetone, nimesulide, orgotein, oxaceprol, paranyline, perisoxal, pifoxime, proquazone, proxazole, and tenidap.

The antibodies and antibody compositions of the invention may be administered alone or in combination with other adjuvants. Adjuvants that may be administered with the antibody and antibody compositions of the invention include, but are not limited to, alum, alum plus deoxycholate (ImmunoAg), MTP-PE (Biocine Corp.), QS21 (Genentech, Inc.), BCG, and MPL. In a specific embodiment, antibody and antibody compositions of the invention are administered in combination with alum. In another specific embodiment, antibody and antibody compositions of the invention are administered in combination with QS-21. Further adjuvants that may be administered with the antibody and antibody compositions of the invention include, but are not limited to, Monophosphoryl lipid immunomodulator, AdjuVax 100a, QS-21, QS-18, CRL1005, Aluminum salts, MF-59, and Virosomal adjuvant technology. Vaccines that may be administered with the antibody and antibody compositions of the invention include, but are not limited to, vaccines directed toward protection against MMR (measles, mumps, rubella), polio, varicella, tetanus/diptheria, hepatitis A, hepatitis B, haemophilus influenzae B, whooping cough, pneumonia, influenza, Lyme's Disease, rotavirus, cholera, yellow fever, Japanese encephalitis, poliomyelitis, rabies, typhoid fever, and pertussis, and/or PNEUMOVAX-23™. Combinations may be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents given first, followed by the second.

In another specific embodiment, antibody and antibody compositions of the invention are used in combination with PNEUMOVAX-23™ to treat, prevent, and/or diagnose infection and/or any disease, disorder, and/or condition associated therewith. In one embodiment, antibody and antibody compositions of the invention are used in combination with PNEUMOVAX-23™ treat, prevent, and/or diagnose any Gram positive bacterial infection and/or any disease, disorder, and/or condition associated therewith. In another embodiment, antibody and antibody compositions of the invention are used in combination with PNEUMOVAX-23™ to treat, prevent, and/or diagnose infection and/or any disease, disorder, and/or condition associated with one or more members of the genus Enterococcus and/or the genus Streptococcus. In another embodiment, antibody and antibody compositions of the invention are used in any combination with PNEUMOVAX-23™ to treat, prevent, and/or diagnose infection and/or any disease, disorder, and/or condition associated with one or more members of the Group B streptococci. In another embodiment, antibody and antibody compositions of the invention are used in combination with PNEUMOVAX-23™ to treat, prevent, and/or diagnose infection and/or any disease, disorder, and/or condition associated with Streptococcus pneumoniae.

In a preferred embodiment, the antibody and antibody compositions of the invention are administered in combination with CD40 ligand (CD40L), a soluble form of CD40L (e.g., AVREND™), bioloigically active fragments, variants, or derivatives of CD40L, anti-CD40L antibodies (e.g., agonistic or antagonistic antibodies), and/or anti-CD40 antibodies (e.g., agonistic or antagonistic antibodies).

In a preferred embodiment, the antibody and antibody compositions of the invention are administered in combination with an NSAID.

In a nonexclusive embodiment, the antibody and antibody compositions of the invention are administered in combination with one, two, three, four, five, ten, or more of the following drugs: NRD-101 (Hoechst Marion Roussel), diclofenac (Dimethaid), oxaprozin potassium (Monsanto), mecasermin (Chiron), T-714 (Toyama), pemetrexed disodium (Eli Lilly), atreleuton (Abbott), valdecoxib (Monsanto), eltenac (Byk Gulden), campath, AGM-1470 (Takeda), CDP-571 (Celltech Chiroscience), CM-101 (CarboMed), ML-3000 (Merckle), CB-2431 (KS Biomedix), CBF-BS2 (KS Biomedix), IL-iRa gene therapy (Valentis), JTE-522 (Japan Tobacco), paclitaxel (Angiotech), DW-166HC (Dong Wha), darbufelone mesylate (Warner-Lambert), soluble TNF receptor 1 (synergen; Amgen), IPR-6001 (Institute for Pharmaceutical Research), trocade (Hoffman-La Roche), EF-5 (Scotia Pharmaceuticals), BIIL-284 (Boehringer Ingelheim), BIIF-1 149 (Boehringer Ingelheim), LeukoVax (Inflammatics), MK-671 (Merck), ST-1482 (Sigma-Tau), and butixocort propionate (WarnerLambert).

In a preferred embodiment, the antibody and antibody compositions of the invention are administered in combination with one, two, three, four, five or more of the following drugs: methotrexate, sulfasalazine, sodium aurothiomalate, auranofin, cyclosporine, penicillamine, azathioprine, an antimalarial drug, cyclophosphamide, chlorambucil, gold, ENBREL™ (Etanercept), anti-TNF antibody, LJP 394 (La Jolla Pharmaceutical Company, San Diego, Calif.) and prednisolone.

In an additional embodiment, antibody and antibody compositions of the invention are administered alone or in combination with one or more intravenous immune globulin preparations. Intravenous immune globulin preparations that may be administered with the antibody and antibody compositions of the invention include, but not limited to, GAMMAR™, IVEEGAM™, SANDOGLOBULIN™, GAMMAGARD S/D™, and GAMIMUNE™. In a specific embodiment, antibody and antibody compositions of the invention are administered in combination with intravenous immune globulin preparations in transplantation therapy (e.g., bone marrow transplant).

CD40 ligand (CD40L), a soluble form of CD40L (e.g., AVREND™), biologically active fragments, variants, or derivatives of CD40L, anti-CD40L antibodies (e.g., agonistic or antagonistic antibodies), and/or anti-CD40 antibodies (e.g., agonistic or antagonistic antibodies).

In an additional embodiment, the antibody and antibody compositions of the invention are administered in combination with cytokines. Cytokines that may be administered with the antibody and antibody compositions of the invention include, but are not limited to, GM-CSF, G-CSF, IL2, IL3, IL4, IL5, IL6, IL7, IL10, IL12, IL13, IL15, anti-CD40, CD40L, IFN-alpha, IFN-beta, IFN-gamma, TNF-alpha, and TNF-beta. In preferred embodiments, antibody and antibody compositions of the invention are administered with TR2. In another embodiment, antibody and antibody compositions of the invention may be administered with any interleukin, including, but not limited to, IL-1alpha, IL-1beta, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, and IL-22. In preferred embodiments, the antibody and antibody compositions of the invention are administered in combination with IL4 and IL10.

In one embodiment, the antibody and antibody compositions of the invention are administered in combination with one or more chemokines. In specific embodiments, the antibody and antibody compositions of the invention are administered in combination with an α(CxC) chemokine selected from the group consisting of gamma-interferon inducible protein-10 (γIP-10), interleukin-8 (IL-8), platelet factor-4 (PF4), neutrophil activating protein (NAP-2), GRO-α, GRO-β, GRO-γ, neutrophil-activating peptide (ENA-78), granulocyte chemoattractant protein-2 (GCP-2), and stromal cell-derived factor-1 (SDF-1, or pre-B cell stimulatory factor (PBSF)); and/or a β(CC) chemokine selected from the group consisting of: RANTES (regulated on activation, normal T expressed and secreted), macrophage inflammatory protein-1 alpha (MIP-1α), macrophage inflammatory protein-1 beta (MIP-1β), monocyte chemotactic protein-1 (MCP-1), monocyte chemotactic protein-2 (MCP-2), monocyte chemotactic protein-3 (MCP-3), monocyte chemotactic protein-4 (MCP-4) macrophage inflammatory protein-1 gamma (MIP-1γ), macrophage inflammatory protein-3 alpha (MIP-3α), macrophage inflammatory protein-3 beta (MIP-3β), macrophage inflammatory protein-4 (MIP-4DC-CK-1/PARC), eotaxin, Exodus, and I-309; and/or the γ(C) chemokine, lymphotactin.

In another embodiment, the antibody and antibody compositions of the invention are administered with chemokine beta-8, chemokine beta-1, and/or macrophage inflammatory protein-4. In a preferred embodiment, the antibody and antibody compositions of the invention are administered with chemokine beta-8.

In an additional embodiment, the antibody and antibody compositions of the invention are administered in combination with an IL-4 antagonist. IL-4 antagonists that may be administered with the antibody and antibody compositions of the invention include, but are not limited to: soluble IL-4 receptor polypeptides, multimeric forms of soluble IL-4 receptor polypeptides; anti-IL-4 receptor antibodies that bind the IL-4 receptor without transducing the biological signal elicited by IL-4, anti-IL4 antibodies that block binding of IL-4 to one or more IL4 receptors, and muteins of IL-4 that bind IL-4 receptors but do not transduce the biological signal elicited by IL-4. Preferably, the antibodies employed according to this method are monoclonal antibodies (including antibody fragments, such as, for example, those described herein).

In an additional embodiment, the antibody and antibody compositions of the invention are administered in combination with fibroblast growth factors. Fibroblast growth factors that may be administered with the antibody and antibody compositions of the invention include, but are not limited to, FGF-1, FGF-2, FGF-3, FGF-4, FGF-5, FGF-6, FGF-7, FGF-8, FGF-9, FGF-10, FGF-11, FGF-12, FGF-13, FGF-14, and FGF-15.

Demonstration of Therapeutic or Prophylactic Utility of a Composition

The compounds of the invention are preferably tested in vitro, and then in vivo for the desired therapeutic or prophylactic activity, prior to use in humans. For example, in vitro assays which can be used to determine whether administration of a specific antibody or composition of the present invention is indicated, include in vitro cell culture assays in which a patient tissue sample is grown in culture, and exposed to or otherwise administered an antibody or composition of the present invention, and the effect of such an antibody or composition of the present invention upon the tissue sample is observed. In various specific embodiments, in vitro assays can be carried out with representative cells of cell types involved in a patient's disorder, to determine if an antibody or composition of the present invention has a desired effect upon such cell types. Preferably, the antibodies or compositions of the invention are also tested in in vitro assays and animal model systems prior to administration to humans.

Antibodies or compositions of the present invention for use in therapy can be tested for their toxicity in suitable animal model systems, including but not limited to rats, mice, chicken, cows, monkeys, and rabbits. For in vivo testing of an antibody or composition's toxicity any animal model system known in the art may be used.

Antibodies or compositions of the invention can be tested for their ability to reduce tumor formation in in vitro, ex vivo and in vivo assays. Antibodies or compositions of the invention can also be tested for their ability to inhibit viral replication or reduce viral load in in vitro and in vivo assays. Antibodies or compositions of the invention can also be tested for their ability to reduce bacterial numbers in in vitro and in vivo assays known to those of skill in the art. Antibodies or compositions of the invention can also be tested for their ability to alleviate of one or more symptoms associated with cancer, an immune disorder (e.g., an inflammatory disease), a neurological disorder or an infectious disease. Antibodies or compositions of the invention can also be tested for their ability to decrease the time course of the infectious disease. Further, antibodies or compositions of the invention can be tested for their ability to increase the survival period of animals suffering from disease or disorder, including cancer, an immune disorder or an infectious disease. Techniques known to those of skill in the art can be used to analyze the function of the antibodies or compositions of the invention in vivo.

Efficacy in treating or preventing viral infection may be demonstrated by detecting the ability of an antibody or composition of the invention to inhibit the replication of the virus, to inhibit transmission or prevent the virus from establishing itself in its host, or to prevent, ameliorate or alleviate the symptoms of disease a progression. The treatment is considered therapeutic if there is, for example, a reduction in viral load, amelioration of one or more symptoms, or a decrease in mortality and/or morbidity following administration of an antibody or composition of the invention.

Antibodies or compositions of the invention can be tested for their ability to modulate the biological activity of immune cells by contacting immune cells, preferably human immune cells (e.g., T-cells, B-cells, and Natural Killer cells), with an antibody or composition of the invention or a control compound and determining the ability of the antibody or compostion of the invention to modulate (i.e, increase or decrease) the biological activity of immune cells. The ability of an antibody or composition of the invention to modulate the biological activity of immune cells can be assessed by detecting the expression of antigens, detecting the proliferation of immune cells (i.e., T-cell proliferation), detecting the activation of signaling molecules, detecting the effector function of immune cells, or detecting the differentiation of immune cells. Techniques known to those of skill in the art can be used for measuring these activities. For example, cellular proliferation can be assayed by ³H-thymidine incorporation assays and trypan blue cell counts. Antigen expression can be assayed, for example, by immunoassays including, but not limited to, competitive and non-competitive assay systems using techniques such as western blots, immunohistochemistry radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays and FACS analysis. The activation of signaling molecules can be assayed, for example, by kinase assays and electrophoretic shift assays (EMSAs). In a preferred embodiment, the ability of an antibody or composition of the invention to induce B-cell proliferation is measured. In another preferred embodiment, the ability of an antibody or composition of the invention to modulate immunoglobulin expression is measured.

Panels/Mixtures

The present invention also provides for mixtures of antibodies (including scFvs and other molecules comprising, or alternatively consisting of, antibody fragments or variants thereof) that specifically bind to TR2 or a fragment or variant thereof, wherein the mixture has at least one, two, three, four, five or more different antibodies of the invention. In specific embodiments, the invention provides mixtures of at least 2, preferably at least 4, at least 6, at least 8, at least 10, at least 12, at least 15, at least 20, or at least 25 different antibodies that specifically bind to TR2 or fragments or variants thereof, wherein at least 1, at least 2, at least 4, at least 6, or at least 10, antibodies of the mixture is an antibody of the invention. In a specific embodiment, each antibody of the mixture is an antibody of the invention.

The present invention also provides for panels of antibodies (including scFvs and other molecules comprising, or alternatively consisting of, antibody fragments or variants thereof) that specifically bind to TR2 or a fragment or variant thereof, wherein the panel has at least one, two, three, four, five or more different antibodies of the invention. In specific embodiments, the invention provides for panels of antibodies that have different affinities for TR2, different specificities for TR2, or different dissociation rates. The invention provides panels of at least 10, preferably at least 25, at least 50, at least 75, at least 100, at least 125, at least 150, at least 175, at least 200, at least 250, at least 300, at least 350, at least 400, at least 450, at least 500, at least 550, at least 600, at least 650, at least 700, at least 750, at least 800, at least 850, at least 900, at least 950, or at least 1000, antibodies. Panels of antibodies can be used, for example, in 96 well plates for assays such as ELISAs.

The present invention further provides for compositions comprising, one or more antibodies (including molecules comprising, or alternatively consisting of antibody fragments or variants of the invention). In one embodiment, a composition of the present invention comprises, one, two, three, four, five, or more antibodies that comprise or alternatively consist of, a polypeptide having an amino acid sequence of any one or more of the VH domains of a one or more of the scFvs referred to in Table 1, or a variant thereof. In another embodiment, a composition of the present invention comprises, one, two, three, four, five, or more antibodies that comprise, or alternatively consist of, a polypeptide having an amino acid sequence of any one or more of the VH CDR1s of a VH domain of one or more of the scFvs referred to in Table 1, or a variant thereof. In another embodiment, a composition of the present invention comprises, one, two, three, four, five or more antibodies that comprise, or alternatively consist of, a polypeptide having an amino acid sequence of any one or more of the VH CDR2s of a VH domain of one or more of the scFvs referred to in Table 1, or a variant thereof. In a preferred embodiment, a composition of the present invention comprises, one, two, three, four, five, or more antibodies that comprise, or alternatively consist of, a polypeptide having an amino acid sequence of any one or more of the VH CDR3s as of a VH domain of one or more of the scFvs referred to in Table 1, or a variant thereof.

Other embodiments of the present invention providing for compositions comprising, one or more antibodies (including molecules comprising, or alternatively consisting of antibody fragments or variants of the invention) are listed below. In another embodiment, a composition of the present invention comprises, one, two, three, four, five, or more antibodies that comprise, or alternative consist of, a polypeptide having an amino acid sequence of any one or more of the VL domains of one or more of the scFvs referred to in Table 1, or a variant thereof. In another embodiment, a composition of the present invention comprises, one, two, three, four, five, or more antibodies that comprise, or alternatively consist of, a polypeptide having an amino acid sequence of any one or more of the VL CDR1s domains of one or more of the scFvs referred to in Table 1, or a variant thereof. In another embodiment, a composition of the present invention comprises, one, two, three, four, five, or more antibodies that comprise, or alternatively consist of, a polypeptide having an amino acid sequence of any one or more of the VL CDR2s of one or more of the scFvs referred to in Table 1, or a variant thereof. In a preferred embodiment, a composition of the present invention comprises, one, two, three, four, five, or more antibodies that comprise, or alternatively consist of, a polypeptide having an amino acid sequence of any one or more of the VL CDR3s domains of one or more of the scFvs referred to in Table 1, or a variant thereof.

Kits

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The present invention provides kits that can be used in the above methods. In one embodiment, a kit comprises an antibody of the invention, preferably a purified antibody, in one or more containers. In an alterative embodiment, a kit comprises an antibody fragment that specifically binds to TR2 polypeptides or fragments or variants thereof. In a specific embodiment, the kits of the present invention contain a substantially isolated TR2 polypeptide or fragment or variant thereof as a control. Preferably, the kits of the present invention further comprise a control antibody which does not react with any, some or all TR2 proteins. In another specific embodiment, the kits of the present invention contain a means for detecting the binding of an antibody to TR2 polypeptides (e.g., the antibody may be conjugated to a detectable substrate such as a fluorescent compound, an enzymatic substrate, a radioactive compound or a luminescent compound, or a second antibody which recognizes the first antibody may be conjugated to a detectable substrate). In specific embodiments, the kit may include a recombinantly produced or chemically synthesized TR2. The TR2 provided in the kit may also be attached to a solid support. In a more specific embodiment the detecting means of the above-described kit includes a solid support to which TR2 is attached. Such a kit may also include a non-attached reporter-labeled anti-human antibody. In this embodiment, binding of the antibody to TR2 can be detected by binding of the said reporter-labeled antibody.

In an additional embodiment, the invention includes a diagnostic kit for use in screening serum containing antigens of the polypeptide of the invention. The diagnostic kit includes a substantially isolated antibody specifically immunoreactive with TR2, and means for detecting the binding of TR2 polypeptides to the antibody. In one embodiment, the antibody is attached to a solid support. In a specific embodiment, the antibody may be a monoclonal antibody. The detecting means of the kit may include a second, labeled monoclonal antibody. Alternatively, or in addition, the detecting means may include a labeled, competing antigen.

In one diagnostic configuration, test serum is reacted with a solid phase reagent having surface-bound TR2 proteins obtained by the methods of the present invention. After TR2 polypeptides bind to a specific antibody, the unbound serum components are removed by washing, reporter-labeled anti-human antibody is added, unbound anti-human antibody is removed by washing, and a reagent is reacted with reporter-labeled anti-human antibody to bind reporter to the reagent in proportion to the amount of bound anti-TR2 antibody on the solid support. Typically, the reporter is an enzyme which is detected by incubating the solid phase in the presence of a suitable fluorometric, luminescent or colorimetric substrate.

The solid surface reagent in the above assay is prepared by known techniques for attaching protein material to solid support material, such as polymeric beads, dip sticks, 96-well plate or filter material. These attachment methods generally include non-specific adsorption of the protein to the support or covalent attachment of the protein, typically through a free amine group, to a chemically reactive group on the solid support, such as an activated carboxyl, hydroxyl, or aldehyde group. Alternatively, streptavidin coated plates can be used in conjunction with biotinylated antigen(s).

Thus, the invention provides an assay system or kit for carrying out this diagnostic method. The kit generally includes a support with surface-bound recombinant TR2, and a reporter-labeled anti-human antibody for detecting surface-bound anti-TR2 antibody.

Gene Therapy

In a specific embodiment, nucleic acids comprising sequences encoding antibodies or functional derivatives thereof, are administered to treat, inhibit or prevent a disease or disorder associated with aberrant expression and/or activity of TR2 proteins and/or its ligands (e.g., TL5 and lymphotoxin-alpha), by way of gene therapy. Gene therapy refers to therapy performed by the administration to a subject of an expressed or expressible nucleic acid. In this embodiment of the invention, the nucleic acids produce their encoded protein that mediates a therapeutic effect.

Any of the methods for gene therapy available in the art can be used according to the present invention. Exemplary methods are described below.

For general reviews of the methods of gene therapy, see Goldspiel et al., Clinical Pharmacy 12:488-505 (1993); Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, TIBTECH 1 l(5):155-215 (1993). Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a preferred aspect, a composition of the invention comprises, or alternatively consists of, nucleic acids encoding an antibody, said nucleic acids being part of an expression vector that expresses the antibody or fragments or chimeric proteins or heavy or light chains thereof in a suitable host. In particular, such nucleic acids have promoters, preferably heterologous promoters, operably linked to the antibody coding region, said promoter being inducible or constitutive, and, optionally, tissue-specific. In another particular embodiment, nucleic acid molecules are used in which the antibody coding sequences and any other desired sequences are flanked by regions that promote homologous recombination at a desired site in the genome, thus providing for intrachromosomal expression of the antibody encoding nucleic acids (Koller and Smithies, Proc. Natl. Acad. Sci. USA 86:8932-8935 (1989); Zijlstra et al., Nature 342:435438 (1989). In specific embodiments, the expressed antibody molecule is an scFv; alternatively, the nucleic acid sequences include sequences encoding both the heavy and light chains, or fragments or variants thereof, of an antibody.

Delivery of the nucleic acids into a patient may be either direct, in which case the patient is directly exposed to the nucleic acid or nucleic acid-carrying vectors, or indirect, in which case, cells are first transformed with the nucleic acids in vitro, then transplanted into the patient. These two approaches are known, respectively, as in vivo or ex vivo gene therapy.

In a specific embodiment, the nucleic acid sequences are directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, J. Biol. Chem. 262:4429-4432 (1987)) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06 180; WO 92/22715; WO92/203 16; WO93/14188, WO 93/20221). Alternatively, the nucleic acid can be introduced intracellularly and incorporated within host cell DNA for expression, by homologous recombination (Koller and Smithies, Proc. Natl. Acad. Sci. USA 86:8932-8935 (1989); Zijlstra et al., Nature 342:435-438 (1989)).

In a specific embodiment, viral vectors that contains nucleic acid sequences encoding an antibody of the invention or fragments or variants thereof are used. For example, a retroviral vector can be used (see Miller et al., Meth. Enzymol. 217:581-599 (1993)). These retroviral vectors contain the components necessary for the correct packaging of the viral genome and integration into the host cell DNA. The nucleic acid sequences encoding the antibody to be used in gene therapy are cloned into one or more vectors, which facilitates delivery of the gene into a patient. More detail about retroviral vectors can be found in Boesen et al., Biotherapy 6:29 1-302 (1994), which describes the use of a retroviral vector to deliver the mdr 1 gene to hematopoietic stem cells in order to make the stem cells more resistant to chemotherapy. Other references illustrating the use of retroviral vectors in gene therapy are: Clowes et al., J. Clin. Invest. 93:644-651(1994); Klein et al., Blood 83:1467-1473 (1994); Salmons and Gunzberg, Human Gene Therapy 4:129-141 (1993); and Grossman and Wilson, Curr. Opin. in Genetics and Devel. 3:110-114 (1993).

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, Current Opinion in Genetics and Development 3:499-503 (1993) present a review of adenovirus-based gene therapy. Bout et al., Human Gene Therapy 5:3-10 (1994) demonstrated the use of adenovirus vectors to transfer genes to the respiratory epithelia of rhesus monkeys. Other instances of the use of adenoviruses in gene therapy can be found in Rosenfeld et al., Science 252:431-434 (1991); Rosenfeld et al., Cell 68:143- 155 (1992); Mastrangeli et al., J. Clin. Invest. 91:225-234 (1993); PCT Publication WO94/12649; and Wang, et al., Gene Therapy 2:775-783 (1995). In a preferred embodiment, adenovirus vectors are used.

Adeno-associated virus (AAV) has also been proposed for use in gene therapy (Walsh et al., Proc. Soc. Exp. Biol. Med. 204:289-300 (1993); U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, Meth. Enzymol. 217:599-718 (1993); Cohen et al., Meth. Enzymol. 217:718-644 (1993); Clin. Pharma. Ther. 29:69-92m (1985)) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a preferred embodiment, the cell used for gene therapy is autologous to the patient.

In an embodiment in which recombinant cells are used in gene therapy, nucleic acid sequences encoding an antibody or fragment thereof are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention (see e.g. PCT Publication WO 94/08598; Stemple and Anderson, Cell 7 1:973-985 (1992); Rheinwald, Meth. Cell Bio. 21A:229 (1980); and Pittelkow and Scott, Mayo Clinic Proc. 71:771 (1986)).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

Having generally described the invention, the same will be more readily understood by reference to the following examples, which are provided by way of illustration and are not intended as limiting.

### EXAMPLES

### Example 1

### Generation of Anti-TR2 Antibodies

General Methods

Rescue of the library.

A library of scFvs is constructed from the RNA of human PBLs as described in WO92/01047 (which is hereby incorporated by reference in its entirety). To rescue phage displaying antibody fragments, approximately 109 E. coli harboring the phagemid are used to inoculate 50 ml of 2×TY containing 1% glucose and 100 micrograms/ml of ampicillin (2×TY-AMP-GLU) and grown to an O.D. of 0.8 with shaking. Five ml of this culture is used to inoculate 50 ml of 2×TY-AMP-GLU, 2×108 TU of delta gene 3 helper (M13 delta gene III, see WO92/01047) are added and the culture incubated at 37?C for 45 minutes without shaking and then at 37?C for 45 minutes with shaking. The culture is centrifuged at 4000 r.p.m. for 10 min. and the pellet resuspended in 2 liters of 2×TY containing 100 micrograms/ml ampicillin and 50 micrograms/ml kanamycin and grown overnight. Phage are prepared as described in WO92/01047.

M13 delta gene III is prepared as follows: M13 delta gene III helper phage does not encode gene III protein, hence the phage(mid) displaying antibody fragments have a greater avidity of binding to antigen. Infectious M13 delta gene III particles are made by growing the helper phage in cells harboring a pUC19 derivative supplying the wild type gene III protein during phage morphogenesis. The culture is incubated for 1 hour at 37?C without shaking and then for a further hour at 37?C with shaking. Cells were spun down (IEC-Centra 8, 4000 revs/min for 10 min), resuspended in 300 ml 2×TY broth containing 100 micrograms ampicillin/ml and 25 micrograms kanamycin/ml (2×TY-AMP-KAN) and grown overnight, shaking at 37?C. Phage particles are purified and concentrated from the culture medium by two PEG-precipitations (Sambrook et al., 1990), resuspended in 2 ml PBS and passed through a 0.45 micrometer filter (Minisart NML; Sartorius) to give a final concentration of approximately 1013 transducing units/ml (ampicillin-resistant clones).

Panning the Library.

Immunotubes (Nunc) are coated overnight in PBS with 4 ml of either 100 micrograms/ml or 10 micrograms/ml of a TR2 polypeptide. Tubes are blocked with 2% Marvel-PBS for 2 hours at 37?C and then washed 3 times in PBS. Approximately 1013 TU of phage is applied to the tube and incubated for 30 minutes at room temperature tumbling on an over and under turntable and then left to stand for another 1.5 hours. Tubes are washed 10 times with PBS 0.1% Tween-20 and 10 times with PBS. Phage are eluted by adding 1 ml of 100 mM triethylamine and rotating 15 minutes on an under and over turntable after which the solution is immediately neutralized with 0.5 ml of 1.0M Tris-HCl, pH 7.4. Phage are then used to infect 10 ml of mid-log E. coli TG1 by incubating eluted phage with bacteria for 30 minutes at 37?C. The E. coli are then plated on TYE plates containing 1% glucose and 100 micrograms/ml ampicillin. The resulting bacterial library is then rescued with delta gene 3 helper phage as described above to prepare phage for a subsequent round of selection. This process is usually repeated for a total of 2-4 rounds of affinity purification.

Characterization of Binders.

Eluted phage from the final rounds of selection are used to infect E. coli HB 2151 and soluble scFv is produced (Marks, et al., 1991) from single colonies for assay. ELISAs are performed with microtiter plates coated with either 10 picograms/ml of the polypeptide of the present invention in 50 mM bicarbonate pH 9.6. Clones positive in ELISA are further characterized by PCR fingerprinting (see e.g., WO92/01047) and then by sequencing.

ELISA Screening for anti-TR2 Specific Antibodies

Antibodies of the present invention may be tested for their ability to bind TR2 using an ELISA.

Plate coating with TR2-Fc.

Fifty μl TR2-Fc solution (2 μg/ml in PBS) are dispensed into individual wells of 96-well plates (Immulon-2, Dynex) sealed with Plate sealers (Advanced Genetic Cat. #48461) and incubated overnight at 40° C. The next day the coating solution is removed, plates are washed 4 times with PBS+0.1% Tween-20 and blocked by incubation with 200 μl of blocking buffer (PBS, 3% BSA) for 1 hour at room temperature

Binding to TR2-Fc (Direct ELISA).

Eight 4-fold serial dilutions of antibodies with concentrations from 10 to 6.1×10⁻⁴ μg/ml are prepared in diluent buffer (PBS, 0.1% Tween-20, 0.1% BSA). Human IgG2 (Sigma, Cat #1-3149) is used as a negative control. Two 50 μl aliquots of each dilution are dispensed into individual wells of TR2-Fc coated plates. The plates are sealed and incubated for 2 hours at room temperature. Plates are washed 4 times with PBST and 50 μl of biotinylated mouse anti human IgG2 (SBA, Cat #9070-08) are dispensed into individual wells (1 μg/ml in diluent buffer). Plates are washed 4 times with PBST and ABC solution is prepared as per manufacturer's instructions and is dispensed into individual wells (50 μl/well in PBST). Plates are sealed and incubated for 30 minutes at room temperature. In the meantime substrate solution is prepared by dissolving 1 tablet of TMB (Sigma, Cat #T3405) in 5 ml of water. After the tablet is dissolved, 5 ml of the substrate buffer (0.1 M NA₂PO₄, 0.05 M Citric acid) and 2 μl of 30% H₂O₂ are added. Plates are washed 4 times with PBST and 100 μl of substrate is added to each well. Plates are incubated for 15 minutes at room temperature and the absorption (at 450 nm) is measured on SpectraMax 3000 (Molecular Devices).

Data analysis.

Data are analyzed using SofMaxPro 3.0. Binding curves in coordinates OD 450 vs. concentration are generated using the four parameter fit model. EC-50 values are calculated automatically as the concentration of the antibody that provides 50% of the maximum binding (maximum binding is chracterized by parameter D in the four parameter fit equation).

### Example 2

### Identification and Cloning of VH and VL Domains

One method to identfy and clone VH and VL domains from cell lines expressing a particular antibody is to perform PCR with VH and VL specific primers on cDNA made from the antibody expressing cell lines. Briefly, RNA is isolated from the cell lines and used as a template for RT-PCR designed to amplify the VH and VL domains of the antibodies expressed by the EBV cell lines. Cells may lysed in the TRIzol® reagent (Life Technologies, Rockville. Md.) and extracted with one fifth volume of chloroform. After addition of chloroform, the solution is allowed to incubate at room temperature for 10 minutes, and the centrifuged at 14,000 rpm for 15 minutes at 4° C. in a tabletop centrifuge. The supernatant is collected and RNA is precipitated using an equal volume of isopropanol. Precipitated RNA is pelleted by centrifuging at 14,000 rpm for 15 minutes at 4° C. in a tabletop centrifuge. Following centrifugation, the supernatant is discarded and washed with 75% ethanol. Follwing washing, the RNA is centrifuged again at 800 rpm for 5 minutes at 4° C. The supernatant is discarded and the pellet allowed to air dry. RNA is the dissolved in DEPC water and heated to 60° C. for 10 minutes. Quantities of RNA can determined using optical density measurements.

cDNA may be synthesized, according to methods well-known in the art, from 1.5-2.5 micrograms of RNA using reverse transciptase and random hexamer primers. cDNA is then used as a template for PCR amplification of VH and VL domains. Alternatively, DNA encoding an scFv, e.g. a vector containing the scFv expression construct, my be used as template material for the following PCR reaction. Primers used to amplify VH and VL genes are shown in Table 4. Typically a PCR reaction makes use of a single 5′ primer and a single 3′ primer. Sometimes, when the amount of available RNA template is limiting, or for greater efficiency, groups of 5′ and/or 3′ primers may be used. For example, sometimes all five VH-5′ primers and all JH3′ primers are used in a single PCR reaction. The PCR reaction is carried out in a 50 microliter volume containing 1×PCR buffer, 2 mM of each dNTP, 0.7 units of High Fidelity Taq polymerse, 5′ primer mix, 3′ primer mix and 7.5 microliters of cDNA. The 5′ and 3′ primer mix of both VH and VL can be made by pooling together 22 pmole and 28 pmole, respectively, of each of the individual primers. PCR conditions are: 96° C. for 5 minutes; followed by 25 cycles of 94° C. for 1 minute, 50° C. for 1 minute, and 72° C. for 1 minute; followed by an extension cycle of 72° C. for 10 minutes. After the reaction is completed, sample tubes were stored 4° C.

TABLE 4

Primer Sequences Used to Amplify VH and VL
domains.

| | | |
|---|---|---|
| Primer name | SEQ ID NO | Primer Aequence (5′-3′) |

| | | |
|---|---|---|
| VH Primers | | |
| Hu VH1-5′ | 9 | CAGGTGCAGCTGGTGCAGTCTGG |
| Hu VH2-5′ | 10 | CAGGTCAACTTAAGGGAGTCTGG |
| Hu VH3-5′ | 11 | GAGGTGCAGCTGGTGGAGTCTGG |
| Hu VH4-5′ | 12 | CAGGTGCAGCTGCAGGAGTCGGG |
| Hu VH5-5′ | 13 | GAGGTGCAGCTGTTGCAGTCTGC |
| Hu VH6-5′ | 14 | CAGGTACAGCTGCAGCAGTCAGG |
| Hu JH1,2-5′ | 15 | TGAGGAGACGGTGACCAGGGTGCC |
| Hu JH3-5′ | 16 | TGAAGAGACGGTGACCATTGTCCC |
| Hu JH4,5-5′ | 17 | TGAGGAGACGGTGACCAGGGTTCC |
| Hu JH6-5′ | 18 | TGAGGAGACGGTGACCGTGGTCCC |
| VL Primers | | |
| Hu Vkappa1-5′ | 19 | GACATCCAGATGACCCAGTCTCC |
| Hu Vkappa2a-5′ | 20 | GATGTTGTGATGACTCAGTCTCC |
| Hu Vkappa2b-5′ | 21 | GATATTGTGATGACTCAGTCTCC |
| Hu Vkappa3-5′ | 22 | GAAATTGTGTTGACGCAGTCTCC |
| Hu Vkappa4-5′ | 23 | GACATCGTGATGACCCAGTCTCC |
| Hu Vkappa5-5′ | 24 | GAAACGACACTCACGCAGTCTCC |
| Hu Vkappa6-5′ | 25 | GAAATTGTGCTGACTCAGTCTCC |
| Hu Vlambda1-5′ | 26 | CAGTCTGTGTTGACGCAGCCGCC |
| Hu Vlambda2-5′ | 27 | CAGTCTGCCCTGACTCAGCCTGC |
| Hu Vlambda3-5′ | 28 | TCCTATGTGCTGACTCAGCCACC |
| Hu Vlambda3b-5′ | 29 | TCTTCTGAGCTGACTCAGGACCC |
| Hu Vlambda4-5′ | 30 | CACGTTATACTGACTCAACCGCC |
| Hu Vlambda5-5′ | 31 | CAGGCTGTGCTCACTCAGCCGTC |
| Hu Vlambda6-5′ | 32 | AATTTTATGCTGACTCAGCCCCA |
| Hu Jkappa1-3′ | 33 | ACGTTTGATTTCCACCTTGGTCCC |
| Hu Jkappa2-3′ | 34 | ACGTTTGATCTCCAGCTTGGTCCC |
| Hu Jkappa3-3′ | 35 | ACGTTTGATATCCACTTTGGTCCC |
| Hu Jkappa4-3′ | 36 | ACGTTTGATCTCCACCTTGGTCCC |
| Hu Jkappa5-3′ | 37 | ACGTTTAATCTCCAGTCGTGTCCC |
| Hu Jlambda1-3′ | 38 | CAGTCTGTGTTGACGCAGCCGCC |
| Hu Jlambda2-3′ | 39 | CAGTCTGCCCTGACTCAGCCTGC |
| Hu Jlambda3--3′ | 40 | TCCTATGTGCTGACTCAGCCACC |
| Hu Jlambda3b-3′ | 41 | TCTTCTGAGCTGACTCAGGACCC |
| Hu Jlambda4-3′ | 42 | CACGTTATACTGACTCAACCGCC |
| Hu Jlambda5-3′ | 43 | CAGGCTGTGCTCACTCAGCCGTC |
| Hu Jlambda6-3′ | 44 | AATTTTATGCTGACTCAGCCCCA |

PCR samples are then electrophoresed on a 1.3% agarose gel. DNA bands of the expected sizes (˜506 base pairs for VH domains, and 344 base pairs for VL domains) can be cut out of the gel and purified using methods well known in the art. Purified PCR products can be ligated into a PCR cloning vector (TA vector from Invitrogen Inc., Carlsbad, Calif.). Individual cloned PCR products can be isolated after transfection of E. coli and blue/white color selection. Cloned PCR products may then be sequenced using methods commonly known in the art.

### Example 3

### T Cell Proliferation Assay

A CD3-induced proliferation assay is performed on purified T cells and is measured by the uptake of 3H-thymidine. The assay is performed as follows. Ninety-six well plates are coated with 100 μl/well of mAb to CD3 (HIT3a, Pharmingen) or isotype-matched control mAb (B33.1) overnight at 4 degrees C. (1 μg/ml in 0.05M bicarbonate buffer, pH 9.5), then washed three times with PBS. Plates may also be coated with anti CD-28 mAb, also available from PharMingen.

T cells may be purified using any standard procedure. For example, T cells may be purified by (positive or negative) selection on magnetic beads, by fluorescence activated cell sorting, or by Ficoll/Hypaque gradient centrifugation from human peripheral blood. Purified cells are added to quadruplicate wells (5×104/well) of mAb coated plates in RPMI containing 10% FCS and penicillin/streptomycin in the presence of LIGHT (e.g., 10 micrograms/milliliter) and/or varying concentrations of antibodies of the invention (total volume 200 microliters). Relevant protein buffer and medium alone are controls. After 48 hr. culture at 37 degrees C., plates are spun for 2 min. at 1000 rpm and 100 microliters of supernatant is removed and stored −20 degrees C. for measurement of IL-2 (or other cytokines) if effect on proliferation is observed. Wells are supplemented with 100 microliters of medium containing 0.5 microCi of 3H-thymidine and cultured at 37 degrees C. for 18-24 hr. Wells are harvested and incorporation of 3H-thymidine used as a measure of proliferation. Anti-CD3 treatment (in the presence or absence of LIGHT) are positive controls for proliferation. IL-2 (100 U/ml) may also be used as a control which enhances proliferation. Control antibody treatment in the absence of LIGHT and any antibodies of the invention) which does not induce proliferation of T cells is used as the negative control for the effects of agonists or antagonists of the invention.

### Example 4

### Effect of Anti-hTR2 Antibodies on Human T Lymphocyte-Mediated zGVHR in Human-PBL/SCID Mice

To test the in vivo effect of anti-TR2 monoclonal antibody (mAb) on T cell activation, a chimeric severe combined immunodeficient mouse engrafted with human peripheral blood (hu-PBL-SCID) model has been developed for xenogeneic graft-versus-host reaction (zGVHR). In this model, donor human T cells are activated by mismatched mouse MHC antigen to against host tissues. It has been suggested that this model may be an excellent tool for investigating the immunosuppression and mechanism of action of biological agents that are specific for human and higher apes and not reactive with lower animals (Tsuchida et al, Transplantation 8:821-7 (1995)). To obtain consistent engraftment, SCID mice were pretreated with anti-asialo GM1 (anti-mouse natural killer cells) antiserum (50 micrograms i.p. day 3) before the i.v. injection of ˜100×10(6) human PBL on day 4. Buffer, control Ig or testing antibodies is given i.v. or i.p. at 1-10 mg/kg/day for 7 days and hu-PBL-SCID mice are sacrificed by exsanguination at day 11 after engraftment with Hu-PBL. Spleens are harvested for (1) splenomegaly by weighting, (2) spontaneous proliferation and cytokine (IFN-γ, GM-GSF) production in culture supernatant for 2-4 days.

It will be clear that the invention may be practiced otherwise than as particularly described in the foregoing description and examples.

Numerous modifications and variations of the present invention are possible in light of the above teachings and, therefore, are within the scope of the appended claims.

The entire disclosure of all publications (including patents, patent applications, journal articles, laboratory manuals, books, or other documents) cited herein are hereby incorporated by reference.

Further, the Sequence Listing submitted herewith, in both computer and paper forms, is hereby incorporated by reference in its entirety.

The entire disclosures (including the specification, sequence listing, and drawings) of International Application No. PCT/US03/10955 filed Apr. 10, 2003 and U.S. Provisional Application No. 60/371,722 filed Apr. 12, 2002 are herein incorporated by reference in their entireties.

1. An isolated antibody or fragment thereof comprising:
(a) an amino acid sequence that is at least 85% identical to the VH domain of the scFv of SEQ ID NO:8;
(b) an amino acid sequence that is at least 85% identical to the VL domain of the scFv of SEQ ID NO:8; or
(c) both (a) and (b);
wherein said antibody or fragment thereof specifically binds a TR2 polypeptide.

2. The antibody or fragment thereof of claim 1 that binds a TR2 polypeptide purified from a cell culture wherein said TR2 polypeptide is encoded by a polynucleotide selected from the group consisting of:
(a) a polynucleotide encoding amino acids 1 to 283 of SEQ ID NO:2;
(b) a polynucleotide encoding amino acids 1 to 185 of SEQ ID NO:4; and
(c) a polynucleotide encoding amino acids 1 to 136 of SEQ ID NO:6.

3. The antibody or fragment thereof of claim 2, wherein the TR2 polypeptide is encoded by (a).

4. The antibody or fragment thereof of claim 2, wherein the TR2 polypeptide is encoded by (b).

5. The antibody or fragment thereof of claim 2, wherein the TR2 polypeptide is encoded by (c).

6. The antibody or fragment thereof of claim 3, which binds the TR2 polypeptide encoded by a polynucleotide encoding amino acids 1 to 185 of SEQ ID NO:4.

7. The antibody or fragment thereof of claim 1 comprising:
(a) an amino acid sequence that is at least 90% identical to the VH domain of the scFv of SEQ ID NO:8;
(b) an amino acid sequence that is at least 90% identical to the VL domain of the scFv of SEQ ID NO:8;
(c) both (a) and (b);
(d) an amino acid sequence that is at least 95% identical to the VH domain of the scFv of SEQ ID NO:8;
(e) an amino acid sequence that is at least 95% identical to the VL domain of the scFv of SEQ ID NO:8; or
(f) both (d) and (e).

8. The antibody or fragment thereof of claim 7 that binds a TR2 polypeptide purified from a cell culture wherein said TR2 polypeptide is encoded by a polynucleotide selected from the group consisting of:
(a) a polynucleotide encoding amino acids 1 to 283 of SEQ ID NO:2;
(b) a polynucleotide encoding amino acids 1 to 185 of SEQ ID NO:4; and
(c) a polynucleotide encoding amino acids 1 to 136 of SEQ ID NO:6.

9. The antibody or fragment thereof of claim 1 comprising:
(a) the amino acid sequence of the VH domain of the scFv of SEQ ID NO:8;
(b) the amino acid sequence of the VL domain of the scFv of SEQ ID NO:8; or
(c) both (a) and (b).

10. The antibody or fragment thereof of claim 9 that binds a TR2 polypeptide purified from a cell culture wherein said TR2 polypeptide is encoded by a polynucleotide selected from the group consisting of:
(a) a polynucleotide encoding amino acids 1 to 283 of SEQ ID NO:2;
(b) a polynucleotide encoding amino acids 1 to 185 of SEQ ID NO:4; and
(c) a polynucleotide encoding amino acids 1 to 136 of SEQ ID NO:6.

11. The antibody or fragment thereof of claim 1, wherein the antibody or fragment thereof is selected from the group consisting of:
(a) a whole immunoglobulin molecule;
(b) an scFv;
(c) a monoclonal antibody;
(d) a human antibody;
(e) a chimeric antibody;
(f) a humanized antibody;
(g) a Fab fragment;
(h) an Fab′ fragment;
(i) an F(ab′)2;
(j) an Fv; and
(k) a disulfide linked Fv.

12. The antibody or fragment thereof of claim 1, which comprises a heavy chain immunoglobulin constant domain selected from the group consisting of:
(a) a human IgM constant domain;
(b) a human IgG1 constant domain;
(c) a human IgG2 constant domain;
(d) a human IgG3 constant domain;
(e) a human IgG4 constant domain; and
(f) a human IgA constant domain.

13. The antibody or fragment thereof of claim 1, which comprises a light chain immunoglobulin constant domain selected from the group consisting of:
(a) a human Ig kappa constant domain; and
(b) a human Ig lambda constant domain.

14. The antibody or fragment thereof of claim 1, wherein the antibody or fragment thereof has a dissociation constant (K_{D}) less than or equal to 10⁻⁹M.

15. The antibody or fragment thereof of claim 14, wherein the antibody or fragment thereof has a dissociation constant (K_{D}) less than or equal to 10⁻¹⁰M.

16. The antibody or fragment thereof of claim 14, wherein the antibody or fragment thereof has a dissociation constant (K_{D}) less than or equal to 10⁻¹¹M.

17. The antibody or fragment thereof of claim 14, wherein the antibody or fragment thereof has a dissociation constant (K_{D}) less than or equal to 10⁻¹²M.

18. The antibody or fragment thereof of claim 1, wherein the antibody or fragment thereof has an off rate less than or equal to 10⁻³/sec.

19. The antibody or fragment thereof of claim 18, wherein the antibody or fragment thereof has an off rate less than or equal to 10⁻/sec.

20. The antibody or fragment thereof of claim 18, wherein the antibody or fragment thereof has an off rate less than or equal to 10⁻⁵/sec.

21. The antibody or fragment thereof of claim 18, wherein the antibody or fragment thereof has an off rate less than or equal to 10⁻⁶/sec.

22. The antibody or fragment thereof of claim 18, wherein the antibody or fragment thereof has an off rate less than or equal to 10⁻⁷/sec.

23. The antibody or fragment thereof of claim 1, wherein the antibody or fragment thereof is conjugated to a detectable label.

24. The antibody or fragment thereof of claim 23, wherein the detectable label is a radiolabel.

25. The antibody or fragment thereof of claim 24, wherein the radiolabel is ¹²⁵I, ¹³¹I, ¹¹¹In, ⁹⁰Y, ⁹⁹Tc, ¹⁷⁷Lu, ¹⁶⁶Ho, or ¹⁵³Sm.

26. The antibody or fragment thereof of claim 23, wherein the detectable label is an enzyme, a fluorescent label, a luminescent label, or a bioluminescent label.

27. The antibody or fragment thereof of claim 1, wherein the antibody or fragment thereof is biotinylated.

28. The antibody or fragment thereof of claim 1, wherein the antibody or fragment thereof is conjugated to a therapeutic or cytotoxic agent.

29. The antibody or fragment thereof of claim 28, wherein the therapeutic or cytotoxic agent is selected from the group consisting of:
(a) an anti-metabolite;
(b) an alkylating agent;
(c) an antibiotic;
(d) a growth factor;
(e) a cytokine;
(f) an anti-angiogenic agent;
(g) an anti-mitotic agent;
(h) an anthracycline;
(i) a toxin; and
(j) an apoptotic agent.

30. The antibody or fragment thereof of claim 1, wherein the antibody or fragment thereof is attached to a solid support.

31. The antibody or fragment thereof of claim 1, wherein the antibody or fragment thereof specifically binds a TR2 polypeptide in a Western blot.

32. The antibody or fragment thereof of claim 1, wherein the antibody or fragment thereof specifically binds a TR2 polypeptide in an ELISA.

33. An isolated cell that produces the antibody or fragment thereof of claim 1.

34. The antibody or fragment thereof of claim 1, wherein the antibody or fragment thereof is an antagonist of a TR2 polypeptide or a fragment thereof.

35. The antibody or fragment thereof of claim 1, wherein the antibody or fragment thereof inhibits, diminishes or abolishes proliferation of cells expressing a TR2 polypeptide.

36. The antibody or fragment thereof of claim 1, wherein the antibody or fragment thereof inhibits, diminishes or abolishes expression of a TR2 polypeptide.

37. The antibody or fragment thereof of claim 1, wherein the antibody or fragment thereof inhibits, diminishes or abolishes binding of a TR2 ligand to a TR2 polypeptide.

38. The antibody or fragment thereof of claim 37, wherein the TR2 ligand is LIGHT.

39. The antibody or fragment thereof of claim 1, wherein the antibody or fragment thereof inhibits, diminishes or abolishes HSV entry into cells expressing a TR2 polypeptide.

40. The antibody or fragment thereof of claim 1, wherein the antibody or fragment thereof inhibits, diminishes or abolishes differentiation of cells expressing a TR2 polypeptide.

41. The antibody or fragment thereof of claim 1, wherein the antibody or fragment thereof is an agonist of a TR2 polypeptide or a fragment thereof.

42. The antibody or fragment thereof of claim 1, wherein the antibody or fragment thereof upregulates expression of a TR2 polypeptide.

43. The antibody or fragment thereof of claim 1, wherein the antibody or fragment thereof stimulates differentiation of cells expressing a TR2 polypeptide.

44. The antibody or fragment thereof of claim 1, wherein the antibody or fragment thereof stimulates proliferation of cells expressing a TR2 polypeptide.

45. An antibody or fragment thereof that competitively inhibits the binding of the antibody or fragment thereof of claim 9 to a TR2 polypeptide.

46. An antibody or fragment thereof that binds the same epitope on a TR2 polypeptide as the antibody or fragment thereof of claim 9.

47. A composition comprising the antibody or fragment thereof of claim 1 and a pharmaceutically acceptable carrier.

48. A method of treating, preventing, or ameliorating a disease or disorder comprising administering to an animal the antibody or fragment thereof of claim 1, wherein the disease or disorder is selected from the group consisting of:
(a) an autoimmune disease;
(b) graft vs. host disease;
(c) transplant rejection;
(d) cancer;
(e) Herpes Simplex Virus infection; and
(f) an immunodeficiency.

49. The method of claim 48, wherein the animal is human.

50. The method of claim 48, wherein the disease or disorder is cancer.

51. The method of claim 50, wherein the antibody or fragment thereof is administered in combination with a chemotherapeutic agent.

52. The method of claim 48, wherein the disease or disorder is an autoimmune disorder.

53. The method of claim 52, wherein the pathology of said autoimmune disorder involves aberrant T cell activity.

54. The method of claim 53, wherein the T cells are selected from the group consisting of:
(a) CD8⁺ T cells; and
(b) T helper 1 cells.

55. The method of claim 48, wherein the disease or disorder is graft vs. host disease.

56. The method of claim 48, wherein the disease or disorder is Herpes Simplex Virus infection.

57. The method of claim 48, wherein the disease or disorder is an immunodeficiency.

58. A method of inhibiting the growth of or killing cells expressing a TR2 polypeptide, comprising administering to an animal in which such inhibition or killing of cells is desired, the antibody or fragment thereof of claim 23 in an amount effective to inhibit the growth or to kill cells expressing a TR2 polypeptide.

59. A method of detecting a TR2 polypeptide in a biological sample comprising:
(a) contacting the biological sample with the antibody or fragment thereof of claim 1; and
(b) detecting said antibody or fragment thereof specifically bound to said TR2 polypeptide.

60. A method of quantitatively measuring expression of a TR2 polypeptide in a biological sample comprising:
(a) contacting the biological sample with the antibody or fragment thereof of claim 1;
(b) measuring said antibody or fragment thereof specifically bound to said TR2 polypeptide; and
(c) comparing the level of said antibody or fragment thereof bound to said TR2 polypeptide with the level of said antibody or fragment thereof bound to a known amount of said TR2 polypeptide.

61. A method for diagnosing or monitoring cancer comprising:
(a) administering to a subject an effective amount of a radiolabeled antibody or fragment thereof of claim 24;
(b) waiting for a time interval following step (a) for permitting said antibody or fragment thereof to preferentially concentrate at sites in the subject where a TR2 polypeptide is expressed;
(c) determining background level; and
(d) detecting said radiolabeled antibody or fragment thereof in the subject, such that detection of said radiolabeled antibody or fragment thereof above the background level indicates that the subject has cancer.

62. A kit comprising the antibody or fragment thereof of claim 1.

63. The kit of claim 62, which further comprises a control antibody.

64. The kit of claim 62, wherein the antibody or fragment thereof is coupled or conjugated to a detectable label.

65. An isolated nucleic acid molecule encoding the antibody or fragment thereof of claim 1.

66. A recombinant vector comprising the isolated nucleic acid molecule of claim 65.

67. The recombinant vector of claim 67, which further comprises at least one nucleotide sequence which regulates the expression of the antibody or fragment thereof encoded by the nucleic acid molecule.

68. A host cell comprising the recombinant vector of claim 66.

69. An isolated antibody or fragment thereof comprising the amino acid sequence of the VHCDR1, VHCDR2, VHCDR3, VLCDR1, VLCDR2, or VLCDR3 of the scFv of SEQ ID NO:8, wherein said antibody or fragment thereof specifically binds a TR2 polypeptide.

70. The antibody or fragment thereof of claim 69 comprising the amino acid sequence of the VHCDR1 region of the scFv of SEQ ID NO:8.

71. The antibody or fragment thereof of claim 69 comprising the amino acid sequence of the VHCDR2 region of the scFv of SEQ ID NO:8.

72. The antibody or fragment thereof of claim 69 comprising the amino acid sequence of the VHCDR3 region of the scFv of SEQ ID NO:8.

73. The antibody or fragment thereof of claim 69 comprising the amino acid sequence of the VLCDR1 region of the scFv of SEQ ID NO:8.

74. The antibody or fragment thereof of claim 69 comprising the amino acid sequence of the VLCDR2 region of the scFv of SEQ ID NO:8.

75. The antibody or fragment thereof of claim 69 comprising the amino acid sequence of the VLCDR3 region of the scFv of SEQ ID NO:8.

76. The antibody or fragment thereof of claim 69 that binds a TR2 polypeptide purified from a cell culture wherein said TR2 polypeptide is encoded by a polynucleotide selected from the group consisting of:
(a) a polynucleotide encoding amino acids 1 to 283 of SEQ ID NO:2;
(b) a polynucleotide encoding amino acids 1 to 185 of SEQ ID NO:4; and
(c) a polynucleotide encoding amino acids 1 to 136 of SEQ ID NO:6.
